(19) United States
(12) Patent Application Publication
(10) Pub. No.: US 2022/0228164 A1
(43) Pub. Date: Jul. 21, 2022

(54) ENGINEERED ANTIGEN PRESENTING CELLS

(71) Applicant: Neogene Therapeutics B.V., Amsterdam (NL)

(72) Inventors: Antonius Nicolaas Maria Schumacher, Bloemendaal (NL); Carsten Linnermann, Amsterdam (NL); Thomas Kuilman, Amstelveen (NL); Gavin M. Bendle, Amsterdam (NL); Jules F.C. Gadiot, Heiloo (NL); Jeroen W.J. van Heijst, Amstelveen (NL); Raquel Gomez-Eerland, Westzaan (NL); Deborah Sophie Schrikkema, Amsterdam (NL); Julia Zoe Walker, Amsterdam (NL); Laura Bies, Haarlem (NL)

(21) Appl. No.: 17/578,955

(22) Filed: Jan. 19, 2022

Related U.S. Application Data
(60) Provisional application No. 63/139,680, filed on Jan. 20, 2021.

(51) Int. Cl. C12N 15/85; C12N 5/074; C12N 5/0789

(52) U.S. Cl. CPC C12N 15/85; C12N 5/0607; A61K 2035/124; C12N 2510/00; C12N 5/0647

(57) ABSTRACT

Provided herein are various engineered antigen presenting cells, methods of making them and methods of using them.

### CROSS REFERENCE TO RELATED APPLICATIONS

This application claims priority to U.S. Prov. App. No. 63/139,680 filed Jan. 20, 2021 entitled “ENGINEERED ANTIGEN PRESENTING CELLS,” which is incorporated by reference in its entirety.

### REFERENCE TO SEQUENCE LISTING

The present application is being filed along with a Sequence Listing in electronic format. The Sequence Listing is provided as a file entitled SeqList_NTBV015A created on Jan. 14, 2022, which is 20,665 bytes in size. The information in the electronic format of the Sequence Listing is incorporated herein by reference in its entirety.

### FIELD

The present disclosure generally relates to both a population of engineered cells and a method of engineering a cell for antigen presentation to T cells.

### DESCRIPTION OF THE RELATED ART

Antigen-presenting cells (APCs), also known as accessory cell, are cells that can display antigen bound by major histocompatibility complex (MHC) proteins along their surfaces. In some cases, APCs process a protein antigen, break it into peptides, and present it along with MHC proteins on their surface. In other cases, APCs are capable of presenting an antigen that has been loaded onto the surface of the cell where the antigen (such as a peptide) binds to the MHC protein.

APCs are involved in both innate and adaptive immune responses, and are found in a variety of tissue types. Classical APCs include dendritic cells, macrophages, Langerhans cells and B cells. APCs play an important role in the immune response, as the functioning of both cytotoxic and helper T cells are dependent on APCs.

### SUMMARY

Disclosed herein are methods of engineering a cell for antigen presentation to T cells. In some embodiments, the method comprises (a) inducing, enhancing and/or maintaining prolonged survival of a cell in vitro, (b) incubating the cell with at least one antigen compound on a continuous basis, and (c) introducing at least one genetic modification within the cell to induce, enhance, maintain and/or modify antigen-presentation by the cell. In some embodiments, the cell comprises a primary human B cell, optionally wherein the primary human B cell is autologous with respect to the T cells or to a TCR presented by the T cells. In some embodiments, the primary human B cell is derived from peripheral blood. In some embodiments, the primary human B cell is derived from a human tissue that contain B cells. In some embodiments, the human tissues comprise tumor tissue, lymph nodes, spleen, cord blood, body fluids, and bone marrow. In some embodiments, the primary human B cell is obtained by differentiation of a precursor cell. In some embodiments, the precursor cell comprises hematopoietic stem cells. In some embodiments, the primary human B cell is obtained by differentiation of induced pluripotent stem cells (iPSC). In some embodiments, the inducing, enhancing and/or maintaining prolonged survival of the cell comprises co-culturing the cell with CD40L-expressing feeder cells. In some embodiments, the method further comprises co-culturing the cell with IL-2, IL-4 and IL-21. In some embodiments, the inducing, enhancing and/or maintaining prolonged survival of the cell comprises expressing BCL-6 and BCL2-like 1 genes in the cell. In some embodiments, the inducing, enhancing and/or maintaining prolonged survival of the cell comprises infecting the cell with EBV. In some embodiments, the method further comprises expressing CD40L gene in the cell. In some embodiments, the inducing, enhancing and/or maintaining prolonged survival of the cell comprises expressing BCL-6, BCL2-like 1, and CD40L genes in the cell. In some embodiments, the BCL-6, BCL2-like 1, and CD40L genes can be on the same expression construct, or different expression construct. In some embodiments, the genes are introduced into the cell through viral transduction, electroporation, squeezing, transfection, site-specific integration, transposons, CRISPR/Cas9, or TALEN. In some embodiments, the prolonged survival of the cell is at least three months in cell culture in vitro. In some embodiments, the at least one antigen compound is encoded by one transgene encoding between one and forty polypeptides. In some embodiments, each polypeptide comprises at least eight or nine amino acids. In some embodiments, the at least one antigen compound is expressed in the cell. In some embodiments, the at least one antigen compound is transiently expressed in the cell.

Also disclosed herein is an engineered cell. In some embodiments, the cell (a) has been adjusted to induce, enhance and/or maintain its survival in vitro; (b) has been incubated with at least one antigen compound, and (c) has at least one genetic modification to induce, enhance, maintain or modify antigen-presentation by the cell. In some embodiments, the cell is adjusted to induce, enhance and/or maintain its survival in vitro comprises expressing BCL-6 and BCL2-like 1 genes in the cell. In some embodiments, the cell is adjusted to induce, enhance and/or maintain its survival in vitro comprises infecting the cell with EBV. In some embodiments, the cell further comprises expressing CD40L gene in the cell. In some embodiments, the genes are introduced into the cell through viral transduction, electroporation, squeezing, transfection, site-specific integration, transposons, CRISPR/Cas9, or TALEN. In some embodiments, the cell survival is at least three months in cell culture in vitro. In some embodiments, the at least one antigen compound is encoded by at least one transgene encoding between one and forty polypeptides. In some embodiments, each polypeptide comprises at least eight amino acids. In some embodiments, the at least one antigen compound is stably expressed in the cell. In some embodiments, the at least one antigen compound is transiently expressed in the cell.

Also disclosed herein is an engineered cell. In some embodiments, the engineered cell comprises (a) a nucleotide sequence for expression of a survival factor, (b) a nucleotide sequence for expression of at least one transgene encoding an antigen, and (c) a nucleotide sequence for expression of CD40L. In some embodiments, the survival factor comprises BCL-6 and/or BCL-XL. In some embodiments, the at least one transgene encoding an antigen encodes for between one and forty polypeptides. In some embodiments, each polypeptide comprises at least eight amino acids. In some embodiments, the nucleotide sequence for expression of CD40L comprises a gene that provides stable expression of CD40L.

Also disclosed herein is a primary human B cell to present antigen to T cells. In some embodiments, the cell comprises (a) a nucleotide sequence providing stable expression of BCL-6 and BCL-XL, (b) a nucleotide sequence providing stable expression of between one and forty polypeptides, each polypeptide encoding at least eight amino acids, and each polypeptide being an antigen, and (c) a nucleotide sequence providing stable expression of CD40L.

Also disclosed herein is a method of antigen presentation. In some embodiments, the method comprises inducing, enhancing and/or maintaining prolonged survival of a cell in vitro, incubating the cell with at least one antigen compound on a continuous basis, and introducing at least one genetic modification within the cell to induce, enhance, maintain and/or modify antigen-presentation by the cell. In some embodiments, the inducing, enhancing and/or maintaining prolonged survival of the cell comprises co-culturing the cell with CD40L-expressing feeder cells. In some embodiments, the method further comprises co-culturing the cell with IL-2, IL-4 and IL-21. In some embodiments, the inducing, enhancing and/or maintaining prolonged survival of the cell comprises expressing BCL-6 and BCL2-like 1 genes in the cell. In some embodiments, the inducing, enhancing and/or maintaining prolonged survival of the cell comprises infecting the cell with EBV. In some embodiments, the method further comprises expressing CD40L gene in the cell. In some embodiments, the BCL-6, BCL2-like 1, or CD40L gene is introduced into the cell through viral transduction, electroporation, squeezing, transfection, site-specific integration, transposons, CRISPR/Cas9, or TALEN. In some embodiments, prolonged survival of the cell is at least three months in cell culture in vitro. In some embodiments, the at least one antigen compound is encoded by one transgene encoding between one and forty polypeptides. In some embodiments, each polypeptide comprises at least eight amino acids. In some embodiments, the at least one antigen compound is stably expressed in the cell. In some embodiments, the at least one antigen compound is transiently expressed in the cell.

Also disclosed herein is a culturing mix. In some embodiments, the culturing mix comprises engineered cells according to any one of the embodiments disclosed herein and a culturing medium. In some embodiments, the antigen compound is expressed from a polynucleotide that is integrated into the cell genome to achieve continuous expression. In some embodiments, expression is at an effective level. In some embodiments, the antigen is introduced once and that the cells can be expanded afterwards, and used multiple times to assess T cells without a need to deliver the antigen a second time.

In some embodiments involving BCL2-like 1, BCL2-like 1 is BCL-xL.

Also disclosed herein is a method of engineering a cell for antigen presentation. In some embodiments, the method comprises (a) inducing, enhancing and/or maintaining prolonged survival of a cell in vitro, (b) incubating the cell with at least one antigen compound for a sufficient duration to present antigen on a surface of the cell, and (c) introducing at least one genetic modification within the cell to induce, enhance, maintain and/or modify antigen-presentation by the cell. In some embodiments, the cell comprises a primary human B cell. In some embodiments, the primary human B cell is derived from peripheral blood. In some embodiments, the primary human B cell is derived from a human tissue that contain B cells. In some embodiments, the human tissues comprise tumor tissue, lymph nodes, spleen, cord blood, body fluids, and bone marrow. In some embodiments, the primary human B cell is obtained by differentiation of a precursor cell. In some embodiments, the precursor cell comprises hematopoietic stem cells. In some embodiments, the primary human B cell is obtained by differentiation of induced pluripotent stem cells (iPSC). In some embodiments, the inducing, enhancing and/or maintaining prolonged survival of the cell comprises co-culturing the cell with CD40L-expressing feeder cells. In some embodiments, the method further comprises co-culturing the cell with IL-2, IL-4 and IL-21. In some embodiments, the inducing, enhancing and/or maintaining prolonged survival of the cell comprises expressing BCL-6 and BCL2-like 1 genes in the cell. In some embodiments, the inducing, enhancing and/or maintaining prolonged survival of the cell comprises infecting the cell with EBV. In some embodiments, the method further comprises expressing CD40L gene in the cell. In some embodiments, the inducing, enhancing and/or maintaining prolonged survival of the cell comprises expressing BCL-6, BCL2-like 1, and CD40L genes in the cell. In some embodiments, the BCL-6, BCL2-like 1, and CD40L genes can be on the same expression construct, or different expression construct. In some embodiments, the genes are introduced into the cell through viral transduction, electroporation, squeezing, transfection, site-specific integration, transposons, CRISPR/Cas9, or TALEN. In some embodiments, the prolonged survival of the cell is at least three months in cell culture in vitro. In some embodiments, the at least one antigen compound is encoded by one transgene encoding between one and forty polypeptides. In some embodiments, each polypeptide comprises at least eight or nine amino acids. In some embodiments, the at least one antigen compound is stably expressed in the cell. In some embodiments, the at least one antigen compound is transiently expressed in the cell. In some embodiments, the at least one antigen compound is electroporated into the cell. In some embodiments, each polypeptide comprises at least eight or nine amino acids. In some embodiments, the antigen compound is a polypeptide between 8 to 25 amino acids long. In some embodiments, the polypeptide is 25 amino acids long. In some embodiments, the antigen is presented to a T cell.

Also disclosed herein is an engineered cell. In some embodiments, the cell (a) has been adjusted to induce, enhance and/or maintain its survival in vitro, (b) has been electroporated with at least one antigen compound or has been engineered to express at least one antigen compound, and (c) has at least one genetic modification to induce, enhance, maintain or modify antigen-presentation by the cell. In some embodiments, the cell is adjusted to induce, enhance and/or maintain its survival in vitro comprises expressing BCL-6 and BCL2-like 1 genes in the cell. In some embodiments, the cell is adjusted to induce, enhance and/or maintain its survival in vitro comprises infecting the cell with EBV. In some embodiments, the cell further comprises expressing CD40L gene in the cell. In some embodiments, the genes are introduced into the cell through viral transduction, electroporation, squeezing, transfection, site-specific integration, transposons, CRISPR/Cas9, or TALEN. In some embodiments, the cell survival is at least three months in cell culture in vitro. In some embodiments, the cell has been engineered to express at least one antigen compound comprises at least one transgene encoding between one and forty polypeptides wherein the polypeptides correspond to the at least one antigen compound. In some embodiments, each polypeptide comprises at least eight amino acids. In some embodiments, the at least one antigen compound is stably expressed in the cell. In some embodiments, the at least one antigen compound is transiently expressed in the cell. In some embodiments, the at least one antigen compound that has been electroporated into the cell is a polypeptide comprising at least eight or nine amino acids. In some embodiments, the polypeptide is between 8 to 25 amino acids long. In some embodiments, the polypeptide is 25 amino acids long. In some embodiments, the engineered cell comprises a primary human B cell. In some embodiments, the primary human B cell is derived from peripheral blood. In some embodiments, the primary human B cell is derived from a human tissue that contain B cells. In some embodiments, the human tissues comprise tumor tissue, lymph nodes, spleen, cord blood, body fluids, and bone marrow. In some embodiments, the primary human B cell is obtained by differentiation of a precursor cell. In some embodiments, the precursor cell comprises hematopoietic stem cells. In some embodiments, the primary human B cell is obtained by differentiation of induced pluripotent stem cells (iPSC).

Also disclosed herein is a culturing mix. In some embodiments, the culturing mix comprises engineered cells according to any one of the embodiments disclosed herein and a culturing medium. In some embodiments, the antigen compound is expressed from a polynucleotide that is integrated into the cell genome to achieve continuous expression. In some embodiments, the antigen compound is expressed from a polynucleotide that is integrated into the cell genome to achieve transient expression. In some embodiments, the antigen compound is expressed from a polynucleotide that is integrated into the cell genome to achieve inducible expression. In some embodiments, the antigen compound is expressed from a polynucleotide that is integrated into the cell via plasmid to achieve continuous expression. In some embodiments, the antigen compound is expressed from a polynucleotide that is integrated into the cell via plasmid to achieve transient expression. In some embodiments, the antigen compound is expressed from a polynucleotide that is integrated into the cell via plasmid to achieve inducible expression. In some embodiments, expression is at an effective level. In some embodiments, the antigen is introduced once. In some embodiments, the antigen is introduced at least once. In some embodiments, the antigen is introduced at least twice. In some embodiments, the antigen is introduced at least three times. In some embodiments, the cells are be expanded afterwards. In some embodiments, the cells can be used to assess T cells. In some embodiments, the cells can be used to assess T cells without a need to deliver the antigen a second time. In some embodiments, the cells can be used multiple times to assess T cells. In some embodiments, the cells can be used multiple times to assess T cells without a need to deliver the antigen a second time. In some embodiments involving BCL2-like 1, wherein BCL2-like 1 is BCL-xL. In some embodiments involving BCL2-like 1, BCL2-like 1 is BCL-xL. In some embodiments, the antigen compound is a TCR antigen compound.

### BRIEF DESCRIPTION OF THE DRAWINGS

FIG. 1 is a flow chart of method of engineering a cell for antigen presentation to T cells.

FIG. 2 shows the features that some embodiments of an engineered cell may include.

FIG. 3 shows some elements of a primary human B cell to present antigen to T cells may have.

FIG. 4 is a flow chart of some steps that may be included in a method of antigen presentation.

FIG. 5A depicts a plot of flow cytometry data of CD3 and CD19 or CD19 and CD20 showing the efficient isolation of B cells from PBMCs.

FIG. 5B depicts a comparison of count (of GFP expressing cells, and thus BCL6/xL) in untransduced (UTD) and transduced. The left panel shows untransduced cells lack GFP (and thus, BCL6/xL expression) whereas the right panel shows that a large fraction of transduced cells are expressing GFP.

FIG. 5C depicts a comparison of BCL6/xL percent over time in CD19+ cells in BCL6/xL UTD and transduced.

FIG. 5D depicts a graph of cell number over time in BCL6/xL UTD vs transduced.

FIG. 5E depicts a graph of cell number over time in EBV LCL. Cells from three donors (BC13, BC14, BC15) were immortalized with EBV infection.

FIG. 6A depicts a plot of flow cytometry data of CD3 and CD19 or CD19 and CD20 showing the efficient isolation of B cells from PBMCs.

FIG. 6B depicts a comparison of count (of GFP expressing cells, and thus Stat5b) in UTD and transduced. The left panel shows untransduced cells lack GFP (and thus, Stat5b expression) whereas the right panel shows that a large fraction of transduced cells are expressing GFP.

FIG. 6C depicts a comparison of Stat5b expression over time in CD19+ cells in Stat5bUTD and transduced.

FIG. 6D depicts a graph of cell number over time in Stat5b UTD vs transduced.

FIG. 7A depicts plots of CD3 and CD20 pre- and post-enrichment.

FIG. 7B depicts a plot of BCL6/BCL-XL vs CD40L in cells transduced with BCL-6/BCL-xL.

FIG. 7C depicts a plot of BCL6/BCL-XL vs CD40L in cells transduced with a combination of BCL-6/BCL-xL and CD40L.

FIG. 7D is a graph depicting % live cells as a fraction of BCL6/xL+ cells over days of post transduction for cells transduced with BCL-6/BCL-xL; cells transduced with BCL-6/BCL-xL and cultured with L cells; and, cells transduced with a combination of BCL-6/BCL-xL and CD40L.

FIG. 7E is a graph depicting the count of live cells over days of post transduction for cells transduced with BCL-6/BCL-xL; cells transduced with BCL-6/BCL-xL and cultured with L cells; and, cells transduced with a combination of BCL-6/BCL-xL and CD40L.

FIG. 7F depicts graphs of TMG expression of BCL6/xL B cells in presence and absence of CD40L co-expression in UTD or Transduced.

FIGS. 8A and 8B are graphs depicting % Ly6G+ of live cells pre and post puromycin selection for three donors.

FIGS. 9A and 9B are bar charts depicting the % CD69 of live cells. FIGS. 9C and 9D are bar charts depicting the % CD69 of TCRb+ cells. FIG. 9A shows a comparison of T cell activation when antigen is presented by BCL6/xL transduced B cells, BCL6/xL transduced B cells with a TMG expressing the antigens, BCL6/xL and CD40L transduced B cells with a TMG expressing the antigens, or BCL6/xL transduced B cells loaded with peptide antigen. FIG. 9B shows a comparison of T cell activation when antigen is presented by EBV immortalized B cells, EBV immortalized B cells with a TMG expressing the antigens, or EBV immortalized B cells loaded with peptide antigen. FIGS. 9C (MHC-I) and 9D (MHC-II) show comparisons of T cell activation when antigen is presented by cells transduced with a combination of BCL-6/BCL-xL and CD40L and with or without LAMP1 sequences with the antigens.

FIGS. 10A-10C show graphs of % CD69+ cells and peptide concentration for different sized peptides loaded onto EBV immortalized B cells. FIG. 10A shows short and long CDK4-17 peptides. FIG. 10B shows short and long CMV-1 peptides. FIG. 10C shows short and long GCN1L1 peptides.

FIG. 10D is a bar chart of % CD69+ cells for various TMG3 peptides loaded as 25-mer peptides with TMG expressed antigen and other controls.

FIG. 10E is a bar chart depicting % CD69+ cells for TMG and minigene presented antigen.

FIGS. 11A-11D is a set of bar charts depicting % CD69+ cells and shows comparisons of T cell activation when antigen is presented by cells transduced with a combination of BCL-6/BCL-xL with and without CD40L expression and with or without LAMP1 sequences with the antigens. FIGS. 11A and 11B show results from use of the 1G4 TCR (Class-I restricted) in separate donors. FIGS. 11C and 11D show results from use of the 5B 8 TCR (Class-II restricted) in separate donors.

FIG. 12A is a plot for CD4 and CD8 in TIL material after rapid expansion.

FIG. 12B are plots depicting CD8 and Live/dead or CD19 and CD20 showing the purity of BCL-6/BCL-xL/CD40L immortalized B cells.

FIG. 12C is a plot depicting BCL6/xL and CD40L expression in the BCL-6/BCL-xL/CD40L immortalized B cells.

FIG. 12D is a bar chart depicting IFN-gamma in TILs co-cultured with TMG expressing BCL-6/BCL-xL/CD40L immortalized B cells.

FIG. 12E is a series of flow cytometry plots of SSC-H and IFN-gamma in TILs co-cultured with TMG expressing BCL-6/BCL-xL/CD40L immortalized B cells.

FIG. 13A is a schematic of various TMG formats.

FIG. 13B are a set of flow histogram plots of TMG expression for UTD vs Transduced, comparing BCL/BCL-XL or BCL/BCL-XL and CD40L.

FIGS. 13C and 13D are a set of bar charts for % CD69, comparing the effect of the presence and absence of CD40L on T cell activation in response to TMG derived antigen.

FIG. 13E is a schematic of various TMG formats.

FIG. 13F are plots comparing TMG expression between UTD, TMG1 Td, and TMG2 Td.

FIG. 13G is a bar chart of % CD69 of T cells in response to antigen presented in two different TMG formats for both Class-I (CDK4-17, CMV-1, and NY-ESO 1G4) and Class-II (NY-ESO 5B8, TP53, and MAGE-A3_R12C9) restricted TCRs.

FIG. 14 is a timeline for some embodiments provided herein.

FIG. 15 is a bar chart of % CD69. FIG. 15 shows a comparison of T cell activation when antigen is presented by cells that have been electroporated with a short or long GCN1L1 peptide, express the peptide from a TMG, and appropriate controls.

FIGS. 16A-16C are a series of bar charts of % CD69. FIGS. 16A and 16B show peptide electroporation and loading respectively for various concentrations and loading times for a long GCN1L1 peptide. FIG. 16C provides the appropriate controls for FIGS. 16A and 16B.

FIGS. 17A-17C are a series of bar charts of % CD69. FIGS. 17A and 17B show peptide electroporation and loading respectively for various concentrations and loading times for a long CMV1 peptide. FIG. 17C provides the appropriate controls for FIGS. 17A and 17B.

FIGS. 18A and 18B are a series of peptide concentration response curves with different times of post-electroporation rest showing % CD69 activation. FIG. 18A shows GCN1L1, FIG. 18B shows CMV1, and FIG. 18C shows MAGE-A3.

FIGS. 19A and 19B are a series of peptide concentration response for different T cell donors showing % CD69 activation. FIG. 19A shows GCN1L1 and FIG. 19B shows CMV1.

FIG. 20 is series of peptide concentration response curves for different T cell donors showing % CD69 activation. FIG. 20 shows MAGE-A3.

FIGS. 21A and 21B are a series of bar charts of % CD69. FIG. 21A shows that a TMG elicits a response in the identified TCRs. FIG. 21B shows that activation elicited by each peptide expressed by the TMG and that the TCRs were selectively activated by a single peptide.

FIGS. 22A and 22B are a series of bar charts of % CD69. FIG. 22A shows that a TMG elicits a response in T cells expressing the identified TCRs. FIG. 22B shows that activation elicited by each peptide encoded by the TMG and that T cells expressing the TCRs were selectively activated by a single peptide.

FIG. 23 is a bar chart of % CD69. FIG. 23 shows for a series of TCRs, only the mutated, but not a wild-type long peptide, can be recognized upon electroporation.

### DETAILED DESCRIPTION

In the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Primary antigen-presenting cells, such as B cells, Dendritic cells and macrophages are frequently used for research. Depending on the mode of antigen-delivery and the specific cell phenotype, antigen-presentation through both the MHC-Class I and II pathway is possible in all of these cell types. However, primary APCs cannot be expanded indefinitely in vitro, and large numbers can possibly be hard to obtain unless significant quantities of human bio-specimens are accessible (e.g. large blood volumes or excised lymph nodes). In order to overcome these challenges, certain types of primary APCs, such as B cells, can be immortalized.

In addition, engineered cell lines (e.g. COS-7, K562, HEK293) are utilized for TCR discovery in several studies. It is possible to obtain large cell numbers when using a cell line. Such systems are commonly used for TCR discovery that involve a limited number of HLA-alleles and/or antigens. However, for TCR discovery on a fully personalized basis (i.e. involving a large and changing repertoire of HLA-alleles) engineered cell lines are much less suited, since MHC genes are the most polymorphic gene loci in humans meaning HLA-engineered cell lines expressing HLA-alleles covering a substantial portion of individuals within the population would need to be created with great effort.

Provided herein are various options for cell lines, as well as method of making and using them, that can be applied, for example, for TCR discovery, characterization and manipulation. In this context, APCs may be autologous or allogeneic to the patient from which TCR sequences have been derived.

In some embodiments, a method of engineering a cell for antigen presentation to T cells is provided. The method can comprise a) inducing, enhancing and/or maintaining prolonged survival of a cell in vitro; b) incubating the cell with at least one antigen compound on a continuous basis; and c) introducing at least one genetic modification within the cell to induce, enhance, maintain and/or modify antigen-presentation by the cell. Thus, in some embodiments, an antigen is associated with the T cells.

In some embodiments, an engineered cell comprises a) a nucleotide sequence for expression of a survival factor; b) a nucleotide sequence for expression of at least one transgene encoding an antigen; and c) a nucleotide sequence for expression of CD40L.

In some embodiments, various embodiments provided herein allow one to start with a lower number of B cells, e.g., less than 100,000, such as less than 90.000, 80,000, 70,000, 60,000, 50,000, or lower. This can be for, a BCL6/xL combination as provided herein. In some embodiments, e.g., for EBV-LCLs, the number can be less than 1×10e8, e.g., 1×10e7, 2×10e6, 1.5×10e6, or lower.

Further provided herein are embodiments in which antigens can be applied to the cells in protein form, rather than as nucleic acids. Thus, in some embodiments, engineered cell lines can refer to cell lines that have had antigenic, exogenous, proteins/peptides added to them, by, for example, electroporation.

Further provided herein are methods of engineering a cell for antigen presentation to T cells. In some embodiments, the method comprises at least one, two, or all three components of: (a) inducing, enhancing and/or maintaining prolonged survival of a cell in vitro, (b) incubating the cell with at least one antigen compound on a continuous basis, and (c) introducing at least one genetic modification within the cell to induce, enhance, maintain and/or modify antigen-presentation by the cell. In some embodiments, the cell is mammalian or mammalian-derived. In some embodiments, the cell is human or human-derived. In some embodiments, the cell comprises a primary human B cell. In some embodiments, the primary human B cell is autologous with respect to the T cells or to a TCR presented by the T cells. In some embodiments, the primary human B cell is derived from peripheral blood. In some embodiments, the primary human B cell is derived from a human tissue that contains B cells. In some embodiments, the human tissue comprises at least one of tumor tissue, lymph nodes, spleen, cord blood, body fluids, and bone marrow. In some embodiments, the primary human B cell is obtained by differentiation of a precursor cell. In some embodiments, the precursor cell comprises hematopoietic stem cells. In some embodiments, the primary human B cell is obtained by differentiation of induced pluripotent stem cells (iPSC). In some embodiments, the one or more of inducing, enhancing and/or maintaining prolonged survival of the cell comprises co-culturing the cell with CD40L-expressing feeder cells. In some embodiments, the method further comprises co-culturing the cell with at least one, two, or all three of IL-2, IL-4 and IL-21. In some embodiments, the at least one of inducing, enhancing and/or maintaining prolonged survival of the cell comprises expressing at least one BCL-6 and/or BCL2-like 1 genes in the cell. In some embodiments, the at least one of inducing, enhancing and/or maintaining prolonged survival of the cell comprises infecting the cell with EBV. In some embodiments, the method further comprises expressing a CD40L gene in the cell. In some embodiments, the at least one of inducing, enhancing and/or maintaining prolonged survival of the cell comprises expressing at least one BCL-6, BCL2-like 1, and/or CD40L genes in the cell. In some embodiments, the at least one BCL-6, BCL2-like 1, and/or CD40L genes are on the same expression construct. In some embodiments, the at least one BCL-6, BCL2-like 1, and/or CD40L genes are on individual expression constructs, respectively. It will be understood that genes can be introduced into the cell using any conventional method known in the art. Nonlimiting examples include viral transduction, electroporation, squeezing, transfection, site-specific integration, transposons, CRISPR/Cas9, and TALEN. In some embodiments, the prolonged survival of the cell is at least about one month, at least about two months, at least about three months, at least about five months, at least about six months, and/or at least about 1 year in cell culture in vitro. In some embodiments, the at least one antigen compound is encoded by an at least one transgene encoding between one and forty polypeptides. In some embodiments, the at least one transgene encodes at least about one polypeptide. In some embodiments, the at least one transgene encodes at least about five polypeptides. In some embodiments, the at least one transgene encodes at least about ten polypeptides. In some embodiments, the at least one transgene encodes at least about twenty polypeptides. In some embodiments, the at least one transgene encodes at least about thirty polypeptides. In some embodiments, the at least one transgene encodes at most about forty polypeptides. In some embodiments, each polypeptide comprises at least eight or nine amino acids. In some embodiments, the at least one antigen compound is expressed in the cell. In some embodiments, the at least one antigen compound is expressed in the cell through induction. In some embodiments, the at least one antigen compound is expressed in the cell constitutively. In some embodiments, the at least one antigen compound is expressed in the cell by genomic transcription. In some embodiments, the at least one antigen compound is expressed in the cell by plasmid. In some embodiments, the at least one antigen compound is transiently expressed in the cell.

Also disclosed herein is an engineered cell. In some embodiments, the cell has been at least one, two, or all three of: (a) adjusted to induce, enhance and/or maintain its survival in vitro; (b) incubated with at least one antigen compound, and (c) has at least one genetic modification that functions to induce, enhance, maintain and/or modify antigen-presentation by the cell. In some embodiments, the cell is adjusted to induce, enhance and/or maintain its survival in vitro by expressing at least one of BCL-6 and/or BCL2-like 1 genes in the cell. In some embodiments, the cell is adjusted to induce, enhance and/or maintain its survival in vitro by infecting the cell with EBV. In some embodiments, the cell further comprises expressing CD40L gene. It will be understood that genes can be introduced into the cell using any conventional method known in the art. Nonlimiting examples include viral transduction, electroporation, squeezing, transfection, site-specific integration, transposons, CRISPR/Cas9, and TALEN. In some embodiments, the prolonged survival of the cell is at least about one month, at least about two months, at least about three months, at least about five months, at least about six months, and/or at least about 1 year in cell culture in vitro. In some embodiments, the at least one antigen compound is encoded by an at least one transgene encoding between one and forty polypeptides. In some embodiments, the at least one transgene encodes at least about one polypeptide. In some embodiments, the at least one transgene encodes at least about five polypeptides. In some embodiments, the at least one transgene encodes at least about ten polypeptides. In some embodiments, the at least one transgene encodes at least about twenty polypeptides. In some embodiments, the at least one transgene encodes at least about thirty polypeptides. In some embodiments, the at least one transgene encodes at most about forty polypeptides. In some embodiments, each polypeptide comprises at least eight or nine amino acids. In some embodiments, the at least one antigen compound is expressed in the cell. In some embodiments, the at least one antigen compound is expressed in the cell through induction. In some embodiments, the at least one antigen compound is expressed in the cell constitutively. In some embodiments, the at least one antigen compound is expressed in the cell by genomic transcription. In some embodiments, the at least one antigen compound is expressed in the cell by plasmid. In some embodiments, the at least one antigen compound is transiently expressed in the cell.

Also disclosed herein is an engineered cell. In some embodiments, the engineered cell comprises at least one, two, or all three of: (a) a nucleotide sequence for expression of a survival factor, (b) a nucleotide sequence for expression of at least one transgene encoding an antigen, and/or (c) a nucleotide sequence for expression of CD40L. In some embodiments, the survival factor comprises at least one of BCL-6 and/or BCL-XL. In some embodiments, the at least one transgene encoding an antigen encodes for between one and forty polypeptides. In some embodiments, the at least one transgene encodes at least about one polypeptide. In some embodiments, the at least one transgene encodes at least about five polypeptides. In some embodiments, the at least one transgene encodes at least about ten polypeptides. In some embodiments, the at least one transgene encodes at least about twenty polypeptides. In some embodiments, the at least one transgene encodes at least about thirty polypeptides. In some embodiments, the at least one transgene encodes at most about forty polypeptides. In some embodiments, each polypeptide comprises at least eight or nine amino acids. In some embodiments, the nucleotide sequence for expression of CD40L comprises a gene that provides stable expression of CD40L. In some embodiments, the nucleotide sequence for expression of CD40L comprises a gene that enhances or improves the expression and/or stability of CD40L.

Also disclosed herein is a primary human B cell to present antigen to T cells. In some embodiments, the cell comprises at least one, two, or all three of: (a) a nucleotide sequence providing stable expression of BCL-6 and/or BCL-XL, (b) a nucleotide sequence providing stable expression of between one and forty polypeptides, each polypeptide encoding at least eight amino acids, and each polypeptide being an antigen, and/or (c) a nucleotide sequence providing stable expression of CD40L.

Also disclosed herein is a method of antigen presentation. In some embodiments, the method comprises at least one of inducing, enhancing and/or maintaining prolonged survival of a cell in vitro, incubating the cell with at least one antigen compound on a continuous basis, and/or introducing at least one genetic modification within the cell to induce, enhance, maintain and/or modify antigen-presentation by the cell. In some embodiments, the at least one of inducing, enhancing and/or maintaining prolonged survival of the cell comprises co-culturing the cell with feeder cells. In some embodiments, the at least one of inducing, enhancing and/or maintaining prolonged survival of the cell comprises co-culturing the cell with CD40L-expressing feeder cells. In some embodiments, the method further comprises co-culturing the cell with at least one, two, or all three of IL-2, IL-4 and/or IL-21. In some embodiments, the at least one of inducing, enhancing and/or maintaining prolonged survival of the cell comprises expressing at least one of BCL-6 and/or BCL2-like 1 genes in the cell. In some embodiments, the at least one of inducing, enhancing and/or maintaining prolonged survival of the cell comprises infecting the cell with EBV. In some embodiments, the method further comprises expressing the CD40L gene in the cell. In some embodiments, at least one of BCL-6, BCL2-like 1, and/or CD40L gene is introduced into the cell through viral transduction, electroporation, squeezing, transfection, site-specific integration, transposons, CRISPR/Cas9, or TALEN. In some embodiments, the prolonged survival of the cell is at least about one month, at least about two months, at least about three months, at least about five months, at least about six months, and/or at least about 1 year in cell culture in vitro. In some embodiments, the at least one antigen compound is encoded by an at least one transgene encoding between one and forty polypeptides. In some embodiments, the at least one transgene encodes at least about one polypeptide. In some embodiments, the at least one transgene encodes at least about five polypeptides. In some embodiments, the at least one transgene encodes at least about ten polypeptides. In some embodiments, the at least one transgene encodes at least about twenty polypeptides. In some embodiments, the at least one transgene encodes at least about thirty polypeptides. In some embodiments, the at least one transgene encodes at most about forty polypeptides. In some embodiments, each polypeptide comprises at least eight or nine amino acids. In some embodiments, the at least one antigen compound is stably expressed in the cell. In some embodiments, the at least one antigen compound is transiently expressed in the cell. In some embodiments, the at least one antigen compound is expressed in the cell through induction. In some embodiments, the at least one antigen compound is expressed in the cell constitutively. In some embodiments, the at least one antigen compound is expressed in the cell by genomic transcription. In some embodiments, the at least one antigen compound is expressed in the cell by plasmid.

Also disclosed herein is a culturing mix. In some embodiments, the culturing mix comprises engineered cells according to any one of the embodiments disclosed herein and a culturing medium. It will be understood that a culturing medium can be any solution suitable for the growth, storage, and/or maintenance of the cells. In some embodiments, the antigen compound is expressed from a polynucleotide that is integrated into the cell genome to achieve continuous expression. In some embodiments, the antigen compound is expressed from a polynucleotide that is integrated into the cell genome to achieve transient expression. In some embodiments, the antigen compound is expressed from a polynucleotide that is integrated into the cell genome to achieve inducible expression. In some embodiments, the antigen compound is expressed from a polynucleotide that is integrated into the cell via plasmid to achieve continuous expression. In some embodiments, the antigen compound is expressed from a polynucleotide that is integrated into the cell via plasmid to achieve transient expression. In some embodiments, the antigen compound is expressed from a polynucleotide that is integrated into the cell via plasmid to achieve inducible expression. In some embodiments, expression is at an effective level. In some embodiments, the antigen is introduced once. In some embodiments, the antigen is introduced at least once. In some embodiments, the antigen is introduced at least twice. In some embodiments, the antigen is introduced at least three times. In some embodiments, the cells are be expanded afterwards. In some embodiments, the cells can be used to assess T cells. In some embodiments, the cells can be used to assess T cells without a need to deliver the antigen a second time. In some embodiments, the cells can be used multiple times to assess T cells. In some embodiments, the cells can be used multiple times to assess T cells without a need to deliver the antigen a second time.

In any of the embodiments disclosed herein involving BCL2-like 1, BCL2-like 1 can be BCL-xL.

Also disclosed herein is a method of engineering a cell for antigen presentation. In some embodiments, the method comprises at least one, two, or all three of: (a) at least one of inducing, enhancing and/or maintaining prolonged survival of a cell in vitro, (b) incubating the cell with at least one antigen compound for a sufficient duration to present antigen on a surface of the cell, and/or (c) introducing at least one genetic modification within the cell to at least one of inducing, enhancing, maintaining and/or modifying antigen-presentation by the cell. In some embodiments, the cell is mammalian or mammalian-derived. In some embodiments, the cell is human or human-derived. In some embodiments, the cell is a B cell or derived from a B cell. In some embodiments, the cell comprises a primary human B cell. In some embodiments, the primary human B cell is derived from peripheral blood. In some embodiments, the primary human B cell is derived from a human tissue that contain B cells. In some embodiments, the human tissues comprise at least one of tumor tissue, lymph nodes, spleen, cord blood, body fluids, and/or bone marrow. In some embodiments, the primary human B cell is obtained by differentiation of a precursor cell. In some embodiments, the precursor cell comprises hematopoietic stem cells. In some embodiments, the primary human B cell is obtained by differentiation of induced pluripotent stem cells (iPSC). In some embodiments, the at least one of inducing, enhancing and/or maintaining prolonged survival of the cell comprises co-culturing the cell with feeder cells. In some embodiments, the at least one of inducing, enhancing and/or maintaining prolonged survival of the cell comprises co-culturing the cell with CD40L-expressing feeder cells. In some embodiments, the method further comprises co-culturing the cell with at least one, two, or all three of IL-2, IL-4 and IL-21. In some embodiments, the at least one of inducing, enhancing and/or maintaining prolonged survival of the cell comprises expressing at least one of BCL-6 and/or BCL2-like 1 genes in the cell. In some embodiments, the at least one of inducing, enhancing and/or maintaining prolonged survival of the cell comprises infecting the cell with EBV. In some embodiments, the method further comprises expressing a CD40L gene in the cell. In some embodiments, the inducing, enhancing and/or maintaining prolonged survival of the cell comprises expressing at least one, two, or all three of BCL-6, BCL2-like 1, and CD40L genes in the cell. In some embodiments, the at least one, two, or all three of BCL-6, BCL2-like 1, and CD40L genes are on the same expression construct. In some embodiments, the at least one, two, or all three of BCL-6, BCL2-like 1, and CD40L genes are on different expression constructs, respectively. In some embodiments, the at least one, two, or all three of BCL-6, BCL2-like 1, and CD40L genes are on a combination of at least two expression constructs, respectively. It will be understood that genes can be introduced into the cell using any conventional method known in the art. Nonlimiting examples include viral transduction, electroporation, squeezing, transfection, site-specific integration, transposons, CRISPR/Cas9, and TALEN. In some embodiments, the prolonged survival of the cell is at least about one month, at least about two months, at least about three months, at least about five months, at least about six months, and/or at least about 1 year in cell culture in vitro. In some embodiments, the at least one antigen compound is encoded by an at least one transgene encoding between one and forty polypeptides. In some embodiments, the at least one transgene encodes at least about one polypeptide. In some embodiments, the at least one transgene encodes at least about five polypeptides. In some embodiments, the at least one transgene encodes at least about ten polypeptides. In some embodiments, the at least one transgene encodes at least about twenty polypeptides. In some embodiments, the at least one transgene encodes at least about thirty polypeptides. In some embodiments, the at least one transgene encodes at most about forty polypeptides. In some embodiments, each polypeptide comprises at least eight or nine amino acids. In some embodiments, the at least one antigen compound is stably expressed in the cell. In some embodiments, the at least one antigen compound is transiently expressed in the cell. In some embodiments, the at least one antigen compound is expressed in the cell through induction. In some embodiments, the at least one antigen compound is expressed in the cell constitutively. In some embodiments, the at least one antigen compound is expressed in the cell by genomic transcription. In some embodiments, the at least one antigen compound is expressed in the cell by plasmid. In some embodiments, the at least one antigen compound is electroporated into the cell. In some embodiments, each polypeptide comprises at least eight or nine amino acids. In some embodiments, the antigen compound is a polypeptide between 8 to 25 amino acids long. In some embodiments, the antigen compound is a polypeptide 8 amino acids long. In some embodiments, the polypeptide is at least about 5 amino acids long. In some embodiments, the polypeptide is at least about 8 amino acids long. In some embodiments, the polypeptide is at least about 10 amino acids long. In some embodiments, the polypeptide is at least about 15 amino acids long. In some embodiments, the polypeptide is at least about 20 amino acids long. In some embodiments, the polypeptide is at most about 25 amino acids long. In some embodiments, the polypeptide is 25 amino acids long. In some embodiments, the antigen is presented to a T cell.

Also disclosed herein is an engineered cell. In some embodiments, the cell has at least one, two, or all three of: (a) been adjusted to at least one of induce, enhance and/or maintain its survival in vitro, (b) been electroporated with at least one antigen compound and/or has been engineered to express at least one antigen compound, and/or (c) has at least one genetic modification to at least one of inducing, enhancing, maintaining or modifying antigen-presentation by the cell. In some embodiments, the cell is adjusted to at least one of induce, enhance and/or maintain its survival in vitro through a process comprising expressing at least one of BCL-6 and/or BCL2-like 1 genes in the cell. In some embodiments, the cell is adjusted to at least one of induce, enhance and/or maintain its survival in vitro through a process comprising infecting the cell with EBV. In some embodiments, the cell further comprises expressing CD40L gene in the cell. It will be understood that genes can be introduced into the cell using any conventional method known in the art. Nonlimiting examples include viral transduction, electroporation, squeezing, transfection, site-specific integration, transposons, CRISPR/Cas9, and TALEN In some embodiments, the prolonged survival of the cell is at least about one month, at least about two months, at least about three months, at least about five months, at least about six months, and/or at least about 1 year in cell culture in vitro. In some embodiments, the at least one antigen compound is encoded by an at least one transgene encoding between one and forty polypeptides. In some embodiments, the at least one transgene encodes at least about one polypeptide. In some embodiments, the at least one transgene encodes at least about five polypeptides. In some embodiments, the at least one transgene encodes at least about ten polypeptides. In some embodiments, the at least one transgene encodes at least about twenty polypeptides. In some embodiments, the at least one transgene encodes at least about thirty polypeptides. In some embodiments, the at least one transgene encodes at most about forty polypeptides. In some embodiments, each polypeptide comprises at least eight or nine amino acids. In some embodiments, the at least one antigen compound is stably expressed in the cell. In some embodiments, the at least one antigen compound is transiently expressed in the cell. In some embodiments, the at least one antigen compound is expressed in the cell through induction. In some embodiments, the at least one antigen compound is expressed in the cell constitutively. In some embodiments, the at least one antigen compound is expressed in the cell by genomic transcription. In some embodiments, the at least one antigen compound is expressed in the cell by plasmid. In some embodiments, the at least one antigen compound is electroporated into the cell. In some embodiments, the at least one antigen compound is a polypeptide comprising at least eight or nine amino acids. In some embodiments, the polypeptide is between 8 to 25 amino acids long. In some embodiments, the polypeptide is 8 amino acids long. In some embodiments, the polypeptide is at least about 5 amino acids long. In some embodiments, the polypeptide is at least about 8 amino acids long. In some embodiments, the polypeptide is at least about 10 amino acids long. In some embodiments, the polypeptide is at least about 15 amino acids long. In some embodiments, the polypeptide is at least about 20 amino acids long. In some embodiments, the polypeptide is at most about 25 amino acids long. In some embodiments, the polypeptide is 25 amino acids long. In some embodiments, the engineered cell is mammalian or mammalian-derived. In some embodiments, the engineered cell is human or human-derived. In some embodiments, the engineered cell is a B cell or derived from a B cell. In some embodiments, the engineered cell comprises a primary human B cell. In some embodiments, the primary human B cell is derived from peripheral blood. In some embodiments, the primary human B cell is derived from a human tissue that contain B cells. In some embodiments, the human tissues comprises at least one of tumor tissue, lymph nodes, spleen, cord blood, body fluids, and/or bone marrow. In some embodiments, the primary human B cell is obtained by differentiation of a precursor cell. In some embodiments, the precursor cell comprises hematopoietic stem cells. In some embodiments, the primary human B cell is obtained by differentiation of induced pluripotent stem cells (iPSC).

Also disclosed herein is a culturing mix. In some embodiments, the culturing mix comprises engineered cells according to any one of the embodiments disclosed herein and a culturing medium. It will be understood that a culturing medium can be any solution suitable for the growth, storage, and/or maintenance of the cells. In some embodiments, the antigen compound is expressed from a polynucleotide that is integrated into the cell genome to achieve continuous expression. In some embodiments, the antigen compound is expressed from a polynucleotide that is integrated into the cell genome to achieve transient expression. In some embodiments, the antigen compound is expressed from a polynucleotide that is integrated into the cell genome to achieve inducible expression. In some embodiments, the antigen compound is expressed from a polynucleotide that is integrated into the cell via plasmid to achieve continuous expression. In some embodiments, the antigen compound is expressed from a polynucleotide that is integrated into the cell via plasmid to achieve transient expression. In some embodiments, the antigen compound is expressed from a polynucleotide that is integrated into the cell via plasmid to achieve inducible expression. In some embodiments, expression is at an effective level. In some embodiments, the antigen is introduced once. In some embodiments, the antigen is introduced at least once. In some embodiments, the antigen is introduced at least twice. In some embodiments, the antigen is introduced at least three times. In some embodiments, the cells are be expanded afterwards. In some embodiments, the cells can be used to assess T cells. In some embodiments, the cells can be used to assess T cells without a need to deliver the antigen a second time. In some embodiments, the cells can be used multiple times to assess T cells. In some embodiments, the cells can be used multiple times to assess T cells without a need to deliver the antigen a second time. In some embodiments, the antigen is introduced once and that the cells can be expanded afterwards, and used multiple times to assess T cells without a need to deliver the antigen a second time. In some embodiments involving BCL2-like 1, BCL2-like 1 is BCL-xL. In some embodiments, the antigen compound is a TCR antigen compound.

### Antigen Presenting Cells (APCs)

Antigen-presenting cells can include two categories: professional and nonprofessional (or “amateur”). Professional APCs include dendritic cells, macrophages, and B cells, whereas nonprofessional APCs that function in antigen presentation for only brief periods include thymic epithelial cells and vascular endothelial cells.

T cells are typically activated before they can divide and perform their function. This can sometimes be achieved by interacting with a professional APC, which presents an antigen recognized by their T cell receptor. T cells cannot recognize (and therefore cannot respond to) “free” or soluble antigens. They can only recognize and respond to antigen that has been processed and presented by cells via carrier molecules like MHC molecules.

APCs can also present foreign and self lipids to T cells and NK cells by using the CD1 family of proteins, which are structurally similar to the MHC class I family

Professional APCs specialize in presenting antigens to T cells. They can be efficient at internalizing antigens, either by phagocytosis, or by receptor-mediated endocytosis, processing the antigen into peptide fragments and then displaying those peptides (bound to an MHC molecule) on their membrane. The T cell recognizes and interacts with the peptide-MHC complex on the membrane of the antigen-presenting cell. An additional co-stimulatory signal is then produced by the antigen-presenting cell, leading to activation of the T cell.

The main types of professional antigen-presenting cells are dendritic cells, macrophages and B cells. B cells can internalize antigen that binds to their B cell receptor and present it as a peptide-MHC complex on the surface of the B cell. Unlike T cells, B cells can recognize soluble antigen for which their B cell receptor is specific. They can then process the antigen and present peptides as peptide-MHC complexes.

### Definitions

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Unless otherwise specified, the definitions provided herein control when the present definitions may be different from other possible definitions.

Unless explained otherwise, all technical and scientific terms used herein have the same meaning as commonly understood to one of ordinary skill in the art to which this disclosure belongs. All HUGO Gene Nomenclature Committee (HGNC) identifiers (IDs) mentioned herein are incorporated by reference in their entirety. Although methods and materials similar or equivalent to those described herein can be used in the practice or testing of the present disclosure, suitable methods and materials are described below. The materials, methods, and examples are illustrative only and not intended to be limiting.

An “antigen-presenting cell” (APC) is a cell that displays antigen at its cell surface. An antigen may be a peptide complexed with major histocompatibility complexes (MHCs, together a peptide-MHC (pMHC) complex) on the cell surfaces, a cell surface protein or any peptide sequence anchored, attached, bound to or associated with the cell surface of an APC. The process of expression of peptides that are associated with class I or class II MHC complexes is known as antigen presentation. T cells may recognize pMHC using their T cell receptors (TCRs), or may recognize other antigens depending on the expression of a suitable receptor molecule, including but not limited to Chimeric Antigen Receptors (CARs), single chain TCRs, single variable domain TCRs and TCR-like antibodies. APCs may process antigens within the cell and present them to T-cells or may be loaded with or binding to extracellular antigens. Almost all cell types can present antigens in some way and can be modified in their antigen-presentation capabilities, including but not limited to, genetic engineering. APCs are found in a variety of tissue types or can be derived from precursor cells or be based on cell lines. Professional antigen-presenting cells, including macrophages, B cells and dendritic cells, can present exogenously acquired foreign antigens to different T cell types as part of inducing a T cell immune response while virus-infected cells (or cancer cells) can present antigens originating inside the cell and thereby become detectable for T cells. In addition to the MHC family of proteins, antigen presentation relies on other specialized signaling molecules on the surfaces of both APCs and T cells.

“T cell receptor” or “TCR” denotes a molecule found on the surface of T cells or T lymphocytes that recognizes antigen bound as peptides to major histocompatibility complex (MHC) molecules. The TCR is composed of two different protein chains (that is, it is a hetero dimer). In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively). This ratio changes during ontogeny and in diseased states (such as leukemia). It also differs between species. Each TCR chain is composed of two extracellular domains: Variable (V) region and a Constant (C) region. The Constant region is proximal to the cell membrane, followed by a transmembrane region and a short cytoplasmic tail, while the Variable region binds to the peptide/MHC complex. The variable domain of both the TCRα and TCRβ chains have three hypervariable complementarity determining regions (CDRs), denoted CDR1, CDR2, and CDR3. In some embodiments, CDR3 is the main antigen-recognizing region. In some embodiments, CDR2 is considered the main MHC-recognizing region. In some embodiments, TCRα chain genes comprise V and J, and TCR chain genes comprise V, D and J gene segments that contribute to TCR diversity. The constant domain of the TCR consists of short connecting sequences in which a cysteine residue forms disulfide bonds, which form a link between the two chains. Notably, alternative TCR formats include but are not limited to, single-chain TCRs, single variable domain TCRs, TCR-like antibodies. Such alternative TCRs may recognize antigen presented by MHC or other antigen formats and may differ in their signaling capabilities from other TCRs.

The term “therapeutic TCR genes” can refer to specific combinations of TCRα and TCRβ chains that mediate a desired functionality, for example, being able to facilitate a host's immune system to fight against a disease. Therapeutic TCR genes can be selected from in vitro mutated TCR chains expressed as recombinant TCR libraries by phage-, yeast- or T cell-display systems or can be obtained from human and animal material. Therapeutic TCR genes can be autologous or allogeneic.

The term “cancer” denotes a malignant neoplasm that has undergone characteristic anaplasia with loss of differentiation, increased rate of growth, invasion of surrounding tissue, and is capable of metastasis. The term “cancer” shall be taken to include a disease that is characterized by uncontrolled growth of cells within a subject. In some embodiments, the terms “cancer” and “tumor” are used interchangeably. In some embodiments, the term “tumor” refers to a benign or non-malignant growth.

As used herein, the term “neo-antigen” refers to an antigen derived from a tumor-specific genomic mutation. For example, a neo-antigen can result from the expression of a mutated protein in a tumor sample due to a non-synonymous single nucleotide mutation, from the expression of alternative open reading frames due to mutation induced frame-shifts or from genomic rearrangement events. Thus, a neo-antigen may be associated with a pathological condition. In some embodiments, “mutated protein” refers to a protein comprising at least one amino acid that is different from the amino acid in the same position of the canonical amino acid sequence. In some embodiments, a mutated protein comprises insertions, deletions, substitutions, inclusion of amino acids resulting from reading frame shifts, fusion of multiple different proteins by genomic rearrangement events or any combination thereof, relative to the canonical amino acid sequence.

“Hematopoietic stem cells” (HSCs) are the stem cells that give rise to different types of blood cells, in lines called myeloid and lymphoid. Lymphoid cells include T cells, B cells, natural killer cells, and innate lymphoid cells. The hematopoietic tissue contains cells with long-term and short-term regeneration capacities and committed multipotent, oligopotent, and unipotent progenitors. Hematopoietic stem cells are found in the bone marrow of adults, especially in the pelvis, femur, and sternum. They are also found in umbilical cord blood and, in small numbers, in peripheral blood. Stem and progenitor cells can be taken from the pelvis, at the iliac crest, using a needle and syringe. The cells can be removed as liquid (to perform a smear to look at the cell morphology) or they can be removed via a core biopsy (to maintain the architecture or relationship of the cells to each other and to the bone). Hematopoietic stem cells cannot be isolated as a pure population, but they can be identified or isolated by the use of flow cytometry where the combination of several different cell surface markers (particularly CD34) are used to separate the Hematopoietic stem cells from the surrounding blood cells.

“Induced pluripotent stem cells” (iPSC) are a type of pluripotent stem cell that can be generated directly from a somatic cell. They can propagate indefinitely, as well as give rise to every other cell type in the body. Since iPSCs can be derived directly from adult tissues, they can be made in a patient-matched manner, which means that each individual could have their own pluripotent stem cell line. These unlimited supplies of autologous cells could be used to generate transplants without the risk of immune rejection. iPSCs are typically derived by introducing products of specific sets of pluripotency-associated genes, or “reprogramming factors”, into a given cell type. Upon introduction of reprogramming factors, cells begin to form colonies that resemble pluripotent stem cells, which can be isolated based on their morphology, conditions that select for their growth, or through expression of surface markers or reporter genes.

CRISPR/Cas9: CRISPR (clustered regularly interspaced short palindromic repeats) is a family of DNA sequences found in the genomes of prokaryotic organisms such as bacteria and archaea. These sequences are derived from DNA fragments of bacteriophages that had previously infected the prokaryote. They are used to detect and destroy DNA from similar bacteriophages during subsequent infections. Hence these sequences play a key role in the antiviral (i.e. anti-phage) defense system of prokaryotes. Cas9 (or “CRISPR-associated protein 9”) is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms. This editing process has a wide variety of applications including basic biological research, development of biotechnology products, and treatment of diseases.

“Transcription activator-like effector nucleases” (TALENs) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to practically any desired DNA sequence, so when combined with a nuclease, DNA can be cut at specific locations. The restriction enzymes can be introduced into cells, for use in gene editing or for genome editing in situ, a technique known as genome editing with engineered nucleases. Alongside zinc finger nucleases and CRISPR/Cas9, TALEN is a prominent tool in the field of genome editing.

A “minigene” is a minimal gene fragment that includes an exon and the control regions necessary for the gene to express itself in the same way as a wild type gene fragment. More complex minigenes can be constructed containing multiple exons and intron(s). Minigenes provide a valuable tool for researchers evaluating splicing patterns both in vivo and in vitro biochemically assessed experiments. Specifically, minigenes are used as splice reporter vectors (also called exon-trapping vectors) and act as a probe to determine which factors are important in splicing outcomes. They can be constructed to test the way both cis-regulatory elements (RNA effects) and trans-regulatory elements (associated proteins/splicing factors) affect gene expression. Minigenes can be used as a transgene to allow expression and antigen presentation of any short peptide sequence in APCs. In some embodiments, minigenes may be additionally engineered to allow efficient class I and II antigen presentation.

As used herein, gene names and protein nomenclature is context dependent, rather than identified by italics or not. Thus, “BCL6” can refer to both the protein and/or the gene, depending upon the context of how it is used (e.g., “encoding for a protein” would be a gene, while “BCL6 is used for . . . ” could be both a gene and/or a protein, while “10 ng of BCL6 is electroporated into cells” would be protein). This shorthand is used to avoid having to restate the same idea in various formats.

“BCL2-like 1” is used herein to denote the general genus. In preferred embodiments, the long isoform is used, BCL2L1 isoform BCL-xL. Where “BCL-xL” is used herein, it will also be appreciated by one of skill in the art, that a subgenus of BCL2-like 1 can also be used/is envisioned as appropriate and that “BCL-XL”, “BCLXL”, “Bcl-xL”, “Bcl-XL”, “bcl-xl” and other similarly meaning terms may be used synonymously in the context of this invention provided that substantially the same function as BCL-xL is maintained. The combination of BCL-6 and BCL-xL may be referred to as “BCL6/XL” or “BCL6/xL” or similar constructions. Thus, the specification contemplates both the genus of options and the individual species (BCL-xL and/or BCL-xS) within the genus BCL2-like 1. In some embodiments, BCL-xL is preferred. Where BCL2-like 1 is used herein, it will be appreciated by one of skill in the art that BCL-xL can be used and is specifically envisioned as a potential species to be used in the context of this invention.

In some embodiments, human genes or proteins are used. A person skilled in the art will appreciate that any variant sequences, including but not limited to homologues from other species may possibly be used in the context of this invention provided it has substantially the same function.

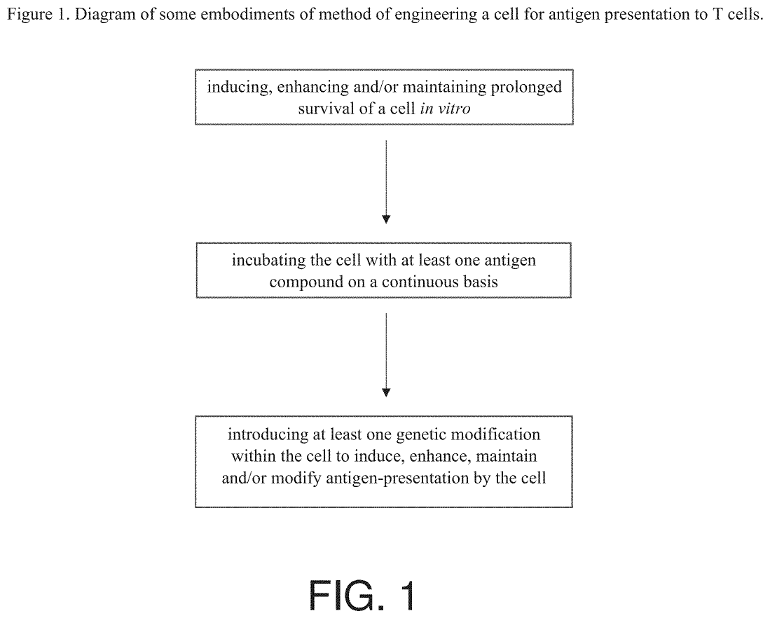

Some embodiments described herein relate to a method of engineering a cell for antigen presentation to T cells. FIG. 1 shows a diagram for some embodiments of method of engineering a cell for antigen presentation to T cells. A method can comprise one or more of the following steps: a) inducing, enhancing and/or maintaining prolonged survival of a cell in vitro; b) incubating the cell with at least one antigen compound on a continuous basis; and c) introducing at least one genetic modification within the cell to induce, enhance, maintain and/or modify antigen-presentation by the cell.

In some embodiments, a cell comprises an autologous primary human B cell. B cells, also known as B lymphocytes, are a type of white blood cell of the lymphocyte subtype. They function in the humoral immunity component of the adaptive immune system by secreting antibodies. Additionally, B cells present antigens (they are also classified as professional antigen-presenting cells (APCs)) and secrete cytokines. In some embodiments, the autologous primary human B cell is derived from peripheral blood. In some embodiments, the autologous primary human B cell is derived from a human tissue that contain B cells. In some embodiments, the human tissues comprise tumor tissue, lymph nodes, spleen, cord blood, body fluids, and bone marrow. In some embodiments, the autologous primary human B cell is autologous to a T cell used in the method. In some embodiments, the autologous primary human B cell is autologous to a TCR used in the method. In some embodiments, a cell comprises a primary human B cell which is not autologous.

In some embodiments, the autologous primary human B cell is or is the same as a cell obtained by differentiation of a precursor cell. In some embodiments, the precursor cell comprises hematopoietic stem cells (HSCs). Here, a precursor cell refers to an immature B cell. B cells develop from hematopoietic stem cells (HSCs). HSCs first differentiate into multipotent progenitor (MPP) cells, then common lymphoid progenitor (CLP) cells. From here, their development into B cells occurs in several stages, each marked by various gene expression patterns and immunoglobulin H chain and L chain gene loci arrangements. B cells undergo two types of selection (positive and negative selections) while developing in the bone marrow to ensure proper development, both involving B cell receptors (BCR) on the surface of the cell. To complete development, immature B cells migrate from the bone marrow into the spleen as transitional B cells, passing through two transitional stages: T1 and T2. Throughout their migration to the spleen and after spleen entry, they are considered T1 B cells. Within the spleen, T1 B cells transition to T2 B cells. T2 B cells differentiate into either follicular (FO) B cells or marginal zone (MZ) B cells depending on signals received through the BCR and other receptors. Once differentiated, they are now considered mature B cells, or naive B cells. Any cells mentioned above before becoming mature B cells are precursor cells. They can be differentiated into mature B cells upon introduction or provision of differentiation factors.

In some embodiments, the autologous primary human B cell is obtained by differentiation of induced pluripotent stem cells (iPSCs). iPSCs are a type of pluripotent stem cell that can be generated directly from a somatic cell. They can propagate indefinitely, and they can give rise to B cells upon introduction of differentiation factors.

In some embodiments, the inducing, enhancing and/or maintaining prolonged survival of the cell comprises co-culturing the cell with CD40L-expressing feeder cells. CD40L, also called CD40 ligand or CD154, is a protein that is primarily expressed on activated T cells and is a member of the TNF superfamily of molecules. It binds to CD40 (protein) on antigen-presenting cells (APCs). The binding leads to many effects depending on the target cell type. In total CD40L has three binding partners: CD40, α5β1 integrin and αIIbβ3. CD40L acts as a costimulatory molecule and is particularly important on a subset of T cells called T follicular helper cells (TFH cells). On TFH cells, CD40L promotes B cell maturation and function by engaging CD40 on the B cell surface and therefore facilitating cell-cell communication. In some embodiments, the CD40-CD40L interaction induces B cell proliferation as well as enhancing their antigen presentation capabilities. In some embodiments, the amino acid sequence for a hCD40L transgene can be, for example,

(SEQ ID NO: 9)
MIETYNQTSPRSAATGLPISMKIFMYLLTVFLITQMIGSALFAVYLHRRL
DKIEDERNLHEDFVFMKTIQRCNTGERSLSLLNCEEIKSQFEGFVKDIML
NKEETKKENSFEMQKGDQNPQIAAHVISEASSKTTSVLQWAEKGYYTMSN
NLVTLENGKQLTVKRQGLYYIYAQVTFCSNREASSQAPFIASLCLKSPGR
FERILLRAANTHSSAKPCGQQSIHLGGVFELQPGASVFVNVTDPSQVSHG
TGFTSFGLLKL*.

In some embodiments, CD40L is provided by co-culture with expressing cells or through direct expression by B cells. In some embodiments, BCL-6/BCL-xL are likewise transgenes. In some embodiments, rh-IL-2 can be used at about 100 IU/mL, rh-IL-4 at about (or BCL2-like 1 more generically) 50 ng/mL and rh-IL-21 at about 50 ng/mL. In some embodiments, IL-2/Il-4 are initially used before cells are cultured in IL-21. In some embodiments, prolonged culture with IL-2/IL-4 may lead to outgrowth of T cells which are residual after the B cell isolation/enrichment. In some embodiments, all of these are provided together (CD40L, IL-4, IL-2 and IL-21, in the amounts and timing described herein (e.g., as in Example 10).

In some embodiments, one can eliminate the need for feeder cells by introducing CD40L directly into the B cells. This can be preferable from an application standpoint and it enhances their antigen-presentation capability. Thus, in some embodiments, any of the embodiments provided herein can be a process that excludes the use of feeder cells (either completely or at a level where the feeder cells actually provide their traditional benefit).

Some embodiments further comprise co-culturing the cell with IL-2, IL-4 and IL-21. Interleukin-2 (IL-2) is an interleukin, a type of cytokine signaling molecule in the immune system. It is a 15.5-16 kDa protein that regulates the activities of white blood cells (leukocytes, often lymphocytes) that are responsible for immunity. IL-2 mediates its effects by binding to IL-2 receptors, which are expressed by lymphocytes. The major sources of IL-2 are activated CD4+ T cells and activated CD8+ T cells. The interleukin 4 (IL4, IL-4) is a cytokine that has many biological roles, including the stimulation of activated B-cell and T-cell proliferation, and the differentiation of B cells into plasma cells. It is a key regulator in humoral and adaptive immunity. Interleukin-21 is a cytokine that has potent regulatory effects on cells of the immune system, including natural killer (NK) cells and cytotoxic T cells. IL-21 mediates its effects by binding to IL-21 receptor, which is expressed on the surface of T, B and NK cells. In some embodiments, typically human protein variants for BCL-6, BCL-xL (or BCL2-like 1 more generically), CD40L, IL-2, IL-4 and IL-21 are most suited to manipulate human B cells, however, that sequences from other species as well as variants of the human protein may be suited as well. In some embodiments, variants can be used, such a soluble CD40L (e.g., to support B cell growth).

In some embodiments, the inducing, enhancing and/or maintaining prolonged survival of the cell comprises co-culturing with CD40L-expressing feeder cells in the presence of IL-2, IL-4 and IL-21. In some embodiments, the combination of IL-2, IL-4 and IL-21 can lead to T cell outgrowth. In some embodiments, IL-2 can be used alone to activate B-cells, then after 48 hours, one can use the combination of these 3 cytokines for 24 hours before one cultures the B cells just with IL-21. In some embodiments, hCD40L-feeders+IL2/IL4/IL21 are used for short-term survival as T cells grow out.

In some embodiments, the inducing, enhancing and/or maintaining prolonged survival of the cell comprises expressing BCL-6 and BCL2-like 1 genes in the cell. In some embodiments, these processes can occur for at least one month or longer, e.g., 4 weeks, 5, 6, 7, 8, 9, 10, 11, 12, or 13 weeks or longer or 3, 4, 5, 6, 7, 8, 9, 10 months or longer. In some embodiments, the long isoform of BCL2-like gene 1—BCL-XL—is used. The term “BCL-XL” is used herein as a short hand reference. Some embodiments further comprise expressing CD40L gene in the cell. The protein encoded by BCL-6 gene has clinical significance in lymphoma. BCL-6 protein is a master transcription factor which leads the differentiation of naive helper T cells in Follicular Helper T cells (TFH cells). It is exclusively present in the B-cells of both healthy and neoplastic germinal centers. B-cell lymphoma-extra large (BCL-XL) is a protein encoded by the BCL2-like 1 gene. BCL-XL is a transmembrane molecule in the mitochondria and plays a role in apoptosis. It will be understood that all such references and embodiments are also contemplated for use with any BCL-like gene 1.

BCL-6/BCL-xL immortalize B cells. Such immortalize B cells require stimulation with CD40L in some frequency. As provided herein, such stimulus can either be provided by CD40L expressing feeder cells or by introduction of CD40L into B cells (see, e.g., Example 10). In some embodiments, if CD40L is introduced into B cells this also improves the antigen-presentation capacity of the B cells (see various Examples). In some embodiments, EBV infection leads to immortalization of infected B cells.

In some embodiments, the inducing, enhancing and/or maintaining prolonged survival of the cell comprises infecting the cell with EBV. The Epstein-Barr virus (EBV), is a herpesvirus. EBV infects B cells of the immune system and epithelial cells. When EBV infects B cells in vitro, lymphoblastoid cell lines eventually emerge that are capable of indefinite growth. The growth transformation of these cell lines is the consequence of viral protein expression. Some embodiments further comprise expressing CD40L gene in the cell. In some embodiments, the inducing, enhancing and/or maintaining prolonged survival of the cell comprises expressing CD40L gene in the cell. In some embodiments, the inducing, enhancing and/or maintaining prolonged survival of the cell comprises co-culturing the cell with CD40L-expressing cells. The prolonged survival of the B cells is the consequence of available CD40-CD40L interaction. In some embodiments, cells can be maintained (for inducing, enhancing, and/or maintaining survival) for at least 0.5, 1, 2, 3, 4, 5, 6, 7, 8, 9, 10 or more months.

In some embodiments, the inducing, enhancing and/or maintaining prolonged survival of the cell comprises expressing BCL-6, BCL2-like 1, and CD40L genes in the cell. In some embodiments, the BCL-6, BCL2-like 1, and CD40L genes can be on the same expression construct, or different expression construct. In some embodiments, expression or stability of one or multiple genes may be regulated by a small molecule siRNA, shRNA or miRNA. In some embodiments, one or multiple genes may be expressed under an exogenous promotor. In some embodiments, one or multiple genes may be expressed under an endogenous promotor.

In some embodiments, the genes are introduced into the cell through viral transduction, electroporation, squeezing, transfection, site-specific integration, transposons, CRISPR/Cas9, or TALEN. The transgenes can be delivered by other methods that lead to stable transgene integration, including other viral gene delivery systems (i.e., lentivirus).

In some embodiments, the prolonged survival of the cell is at least three months in cell culture in vitro. In some embodiments, the prolonged survival of the cell is 0.5, 1, 2, 3, 4, 5, 6, 7, 8, 9, 10, 11, 12, 13, 14, 15, 16, 17, 18, 19, 20, 21, 22, 23, or 24 months or longer in cell culture in vitro.

In some embodiments, the at least one antigen compound is encoded by one transgene encoding between one and forty polypeptides. In some embodiments, the transgene encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95 or 100 polypeptides, where the polypeptides can be the same or different. In some embodiments, each polypeptide comprises at least eight amino acids. In some embodiments, each polypeptide comprises 9, 10, 11, 12, 13, 14, 15, 16, 17, 18, 19, 20, 21, 22, 23, 24, 25, 26, 27, 28, 29, 30, 50, 75, 100, 200, 300, 400, 500, 600, 700, 800, 900, 1000 or more amino acids. In some embodiments, the order of polypeptides in the transgene is chosen such that each antigen is processed and presented. In some embodiments, such order of polypeptides is predicted by in silico algorithms. In some embodiments, the order of polypeptides is determined experimentally. In some embodiments, multiple orders of polypeptides are used in the same or different transgenes to avoid positional effects on polypeptide antigen-presentation. In some embodiments, the transgene encodes longer parts of a protein or a full protein sequence. In some embodiments, the transgene may encode one or more mutated protein sequences found within a tumor. In some embodiments, the transgene may encode an alternative open reading frame. In some embodiments, the transgene may encode a genomic fusion side. In some embodiments, the transgene may include variants of the same polypeptide. In some embodiments, the transgene encodes a part of the human exome or part of a human genomic locus. In some embodiments, the transgene encodes one or multiple antigens from a cancer-associated virus. In some embodiments, the transgene encodes an antigen used to identify or characterize TCRs for cancer therapy or another human disease. In some embodiments, the transgene contains LAMP-1 signaling, transmembrane and truncated cytosolic sequences to target the antigen into the MHC-class II presentation pathway. In some embodiments, the transgene contains other sequences that allow efficient targeting of the antigen into the MHC-class II presentation pathway, for instance by inducing trafficking to the cell membrane or endocytic vesicles. In some embodiments, the various regions encoding polypeptide are separated by spacer sequence or a cleavage site, including but not limited to viral 2A sequence and protease cleavage sites. In some embodiments, other sequences are added to the transgene to increase stability of the encoded polypeptide. In some embodiment, other sequences are included in the transgene to increase the efficiency of MHC-class I or II related antigen presentation. In some embodiments, the transgene is expressed under an exogenous promotor. In some embodiments, the transgene is expressed under an endogenous promotor. In some embodiments, expression of the transgene is regulated by a small molecule, siRNA, shRNA or miRNA in bi- or polycistronic transgene cassette.

Antigen can be delivered to APCs in different formats. First, co-incubation of APCs with a polypeptide of variable length leads to antigen-presentation by APCs. Depending on the length of the peptide, presentation preferably occurs by the MHC-class I (approx. 9-11 amino acids) or MHC-class II (approx. 14-25 amino acids) presentation pathway. Notably, shorter (9-11 aa) peptides typically do not bind to MHC-class II molecules and longer peptides (14+ amino acids) are not always reliably processed and presented by the MHC-class I presentation pathway. Therefore, co-incubation with a defined peptide length is only suited to evaluate antigen presentation by either the MHC-class I or II pathway, but not both, unless electroporation with longer peptides is used.

Second, full-length recombinant protein has been used as antigen in several studies. Provided that the recombinant protein is efficiently endocytosed by APCs (e.g. by targeting endocytosis receptors on APCs), it will lead to both MHC-class I and II antigen presentation. However, given the involved production process, full-length recombinant protein is not suitable for studies that involve large numbers of different antigens, e.g. TCR discovery against mutated proteins in tumors which will vary between each tumor.

Third, antigen can be transiently expressed in APCs using either DNA-plasmids or mRNA. Given the ease of accessibility, antigen delivery by nucleic acids is particularly suitable when studying many different antigens. The main disadvantage of the method is the transient and variable expression of the antigen after transfection into APCs, leading to heterogeneous APC populations. Stable transduction with the antigen provides a possible solution but can be difficult to achieve with high efficiency and without detrimental loss of cell viability for many primary cell types without immortalization.

In some embodiments, the at least one antigen compound is stably expressed in the cell. The antigen encoding transgene can be delivered by any method that leads to stable transgene integration, including viral gene delivery systems (retro or lentivirus) as well as non-viral gene delivery methods such as site-specific integration, CRISPR/Cas9, TALEN, and Transposon-based vectors. In some embodiments, the process can include: viral transduction, electroporation, squeezing, transfection, site-specific integration, transposons, CRISPR/Cas9, or TALEN. Stable expression of an antigen allows one to assess antigen processing and presentation. Furthermore, stable expression of saturating amounts of antigen minimizes heterogenous antigen expression within the APC population (which would negatively impact sensitivity of TCR discovery). In addition, stable expression also eases the generation of high numbers of APCs. In some embodiments, the transgene is stably expressed under an exogenous promotor. In some embodiments, the transgene is stably expressed under an endogenous promotor. In some embodiments, the promotor for stable expression is chosen to allow control of the expression level.

In some embodiments, at least one antigen compound is transiently expressed in the cell. In some embodiments, the mix will contain at least one cell and at least one exogenous nucleic acid sequence encoding at least one antigen. In some embodiments, the mix will contain at least one cell and at least one polypeptide sequence encoding at least one antigen. In some embodiments, the cells are cultured in culture media. The transient expression in the cells can be achieved using either DNA-plasmids or mRNA. Delivering antigens by transiently expressing nucleic acids is particularly suitable when studying many different antigens. The main disadvantage of the method is that the transient and variable expression of the antigen after transfection into APCs leads to heterogeneous APC populations. In some embodiments, the DNA plasmid or mRNA encode a marker in combination with the antigen in order to enable to detect and select cells expressing the antigen compound.

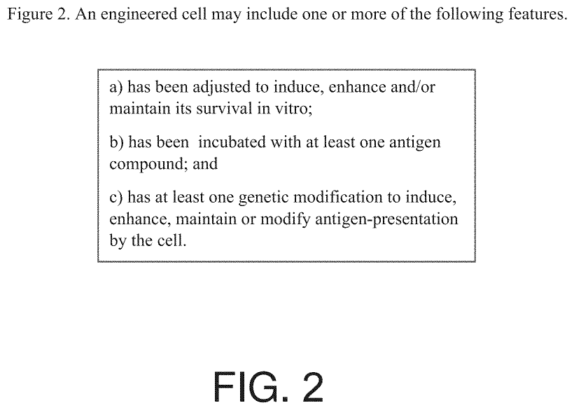

FIG. 2 shows some embodiments that relate to an engineered cell. In some embodiments, a cell includes one or more of the following features: a) has been adjusted to induce, enhance and/or maintain its survival in vitro; b) has been incubated with at least one antigen compound; and c) has at least one genetic modification to induce, enhance, maintain or modify antigen-presentation by the cell. The cells can be primary antigen-presenting cells, such as B cells, Dendritic cells, or macrophages. The cells can be tumor cells. The cells can be pathogen-infected cells. The cells can also be cell lines, COS-7, K562, or HEK293.

In some embodiments, the process is adjusted to induce, enhance and/or maintain its survival in vitro comprises expressing BCL-6 and BCL2-like 1 genes in the cell. In some embodiments, the process is adjusted to induce, enhance and/or maintain its survival in vitro comprises infecting the cell with EBV. Some embodiments further comprise expressing CD40L gene in the cell. In some embodiments, the expression of CD40L serves to induce, enhance and/or maintain its survival in vitro.

In some embodiments, the genes are introduced into the cell through viral transduction, electroporation, squeezing, transfection, site-specific integration, transposons, CRISPR/Cas9, or TALEN. The transgenes can be delivered by other methods that lead to stable transgene integration, including other viral gene delivery systems (i.e., lentivirus).

In some embodiments, the cell survival is at least three months in cell culture in vitro. In some embodiments, the prolonged survival of the cell is 0.5, 1, 2, 3, 4, 5, 6, 7, 8, 9, 10, 11, 12, 13, 14, 15, 16, 17, 18, 19, 20, 21, 22, 23, or 24 months in cell culture in vitro.

In some embodiments, the at least one antigen compound is encoded by at least one transgene encoding between one and forty polypeptides. In some embodiments, the transgene encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 polypeptides, where the polypeptides can be the same or different. In some embodiments, each polypeptide comprises at least eight amino acids. In some embodiments, each polypeptide comprises 9, 10, 11, 12, 13, 14, 15, 16, 17, 18, 19, 20, 21, 22, 23, 24, 25, 26, 27, 28, 29, 30, 75, 100, 200, 300, 400, 500, 600, 700, 800, 900, 1000 or more amino acids amino acids.

In some embodiments, the at least one antigen compound is stably expressed in the cell. The antigen encoding transgene can be delivered by any methods that lead to stable transgene integration, including viral gene delivery systems (retro or lentivirus) as well as non-viral gene delivery methods such as site-specific integration, CRISPR/Cas9, TALEN, and Transposon-based vectors. In some embodiments, the process can include viral transduction, electroporation, squeezing, transfection, site-specific integration, transposons, CRISPR/Cas9, or TALEN. In some embodiments, the at least one antigen compound is transiently expressed in the cell.

In some embodiments, an engineered cell comprises a) a nucleotide sequence for expression of a survival factor; b) a nucleotide sequence for expression of at least one transgene encoding an antigen; and c) a nucleotide sequence for expression of CD40L. In some embodiments, the survival factor comprises BCL-6 and/or BCL-XL. In some embodiments, at least one transgene encoding an antigen encodes for between one and forty polypeptides. In some embodiments, the transgene encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 polypeptides, where the polypeptides can be the same or different. In some embodiments, each polypeptide comprises eight or nine amino acids. In some embodiments, each polypeptide comprises 8, 9, 10, 11, 12, 13, 14, 15, 16, 17, 18, 19, 20, 21, 22, 23, 24, 25, 26, 27, 28, 29, 30, 50, 75, 100, 200, 300, 400, 500, 600, 700, 800, 900, 1000 or more amino acids.

In some embodiments, the nucleotide sequence for expression of CD40L comprises a gene that provides stable expression of CD40L.

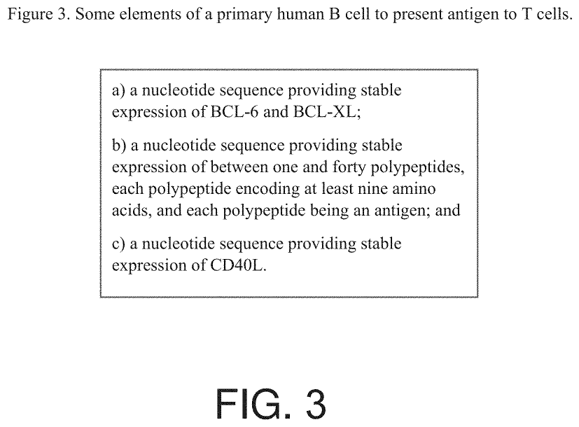

In some embodiments, as shown in FIG. 3, a primary human B cell to present antigen to T cells, the cell comprising: a) a nucleotide sequence providing stable expression of BCL-6 and BCL-XL (e.g., SEQ ID 1); b) a nucleotide sequence providing stable expression of between one and forty polypeptides, each polypeptide encoding at least eight or nine amino acids, and each polypeptide being an antigen; and c) a nucleotide sequence providing stable expression of CD40L. In some embodiments, the nucleotide sequence encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 polypeptides, where the polypeptides can be the same or different. In some embodiments, each polypeptide comprises 9, 10, 11, 12, 13, 14, 15, 16, 17, 18, 19, 20, 21, 22, 23, 24, 25, 26, 27, 28, 29, 30, 50, 75, 100, 200, 300, 400, 500, 600, 700, 800, 900, 1000 or more amino acids.

Amino acid sequence for a BCL6-T2A-BCL-xL-P2A-GFP
transgene (SEQ ID NO: 1):
MASPADSCIQFTRHASDVLLNLNRLRSRDILTDVVIVVSREQFRAHKTV
LMACSGLFYSIFTDQLKCNLSVINLDPEINPEGFCILLDFMYTSRLNLR
EGNIMAVMATAMYLQMEHVVDTCRKFIKASEAEMVSAIKPPREEFLNSR
MLMPQDIMAYRGREVVENNLPLRSAPGCESRAFAPSLYSGLSTPPASYS
MYSHLPVSSLLFSDEEFRDVRMPVANPFPKERALPCDSARPVPGEYSRP
TLEVSPNVCHSNIYSPKETIPEEARSDMHYSVAEGLKPAAPSARNAPYF
PCDKASKEEERPSSEDEIALHFEPPNAPLNRKGLVSPQSPQKSDCQPNS
PTESCSSKNACILQASGSPPAKSPTDPKACNWKKYKFIVLNSLNQNAKP
EGPEQAELGRLSPRAYTAPPACQPPMEPENLDLQSPTKLSASGEDSTIP
QASRLNNIVNRSMTGSPRSSSESHSPLYMHPPKCTSCGSQSPQHAEMCL
HTAGPTFPEEMGETQSEYSDSSCENGAFFCNECDCRFSEEASLKRHTLQ
THSDKPYKCDRCQASFRYKGNLASHKTVHTGEKPYRCNICGAQFNRPAN
LKTHTRIHSGEKPYKCETCGARFVQVAHLRAHVLIHTGEKPYPCEICGT
RFRHLQTLKSHLRIHTGEKPYHCEKCNLHFRHKSQLRLHLRQKHGAITN
TKVQYRVSATDLPPELPKACGSGEGRGSLLTCGDVEENPGPMSQSNREL
VVDFLSYKLSQKGYSWSQFSDVEENRTEAPEGTESEMETPSAINGNPSW
HLADSPAVNGATGHSSSLDAREVIPMAAVKQALREAGDEFELRYRRAFS
DLTSQLHITPGTAYQSFEQVVNELFRDGVNWGRIVAFFSFGGALCVESV
DKEMQVLVSRIAAWMATYLNDHLEPWIQENGGWDTFVELYGNNAAAESR
KGQERFNRWFLTGMTVAGVVLLGSLFSRKEFGSGATNFSLLKQAGDVEE
NPGPMVSKGEELFTGVVPILVELDGDVNGHKFSVSGEGEGDATYGKLTL
KFICTTGKLPVPWPTLVTTLTYGVQCFSRYPDHMKQHDFFKSAMPEGYV
QERTIFFKDDGNYKTRAEVKFEGDTLVNRIELKGIDFKEDGNILGHKLE
YNYNSHNVYIMADKQKNGIKVNFKIRHNIEDGSVQLADHYQQNTPIGDG
PVLLPDNHYLSTQSALSKDPNEKRDHMVLLEFVTAAGITLGMDELYK*

Some embodiments can include the engineered cells described herein and a culturing medium.

### Additional Embodiments

There are several shortcomings of various technologies that are applied to screen several thousands to millions of T cells/TCRs. Several TCR discovery studies are based on the use of recombinant MHC-multimer based reagents (Tsuji et al. Cancer Immunol Res 2018; Peng et al. Cell Reports 2019; Spindler et al. Nat Biotech 2020) and do not use any APCs in their discovery process. Such reagents are only available for certain HLA-alleles and do not provide conclusive proof for antigen-processing and presentation by cells. Also, such methods allow for screening biophysical properties of the TCR, but do not allow screening based on functional properties such as activation of a T cell. Other studies utilize primary human APCs transiently expressing antigen delivered in the form of peptide, mRNA or plasmid (Tran et al. Science 2014; Tran et al. Science 2015; Gros et al. Nat Med 2016). Such delivery methods lead to heterogenous antigen levels on APCs and do not provide a solution as to how to track antigen expression/presentation by APCs over time (i.e. whether an APC still presents antigen hours or days after loading). Furthermore, large antigen amounts, e.g. mRNA, may be required to generate large numbers of antigen-loaded APCs. Thus, these methods are not suited if large numbers of APCs with homogeneous and sustained antigen expression levels are required. Further, engineered APCs have been used in a recent study (Kula et al. Cell 2019). There, APCs were transduced with an antigen-library of interest and a reporter system to mark APCs that present antigen recognized by T cells. While the antigen is stably expressed and large numbers of APCs can be obtained, this system is not suited for fully personalized TCR discovery since HLA-alleles of interest need to be introduced into the engineered APC for every individual. Methods using a cell line engineered to express one or more defined HLA-alleles and to be capable to present antigen (Butler et al. Immunol Rev 2014) are likewise unsuited.

In some embodiments, the APCs described herein can have at least one of the following three advantages: first, the APC can be easily expanded to large cell numbers and maintained for prolonged periods in cell culture. Second, the APC can stably express antigen(s) of interest to achieve high and sustained antigen presentation, with reduced heterogeneity within the cell population. Third, the APC can present putative T cell epitopes to focus TCR discovery on particular aspects of interest (MHC-class I or class II pathways, specific HLA-alleles and activation thresholds). In some embodiments, the APC can have one or more genetic modifications to allow more efficient antigen presentation. In some embodiments, the APC include a LAMP1 targeting sequence. In some embodiments, the APC described herein can have one of the following advantages: first, they express an antigen to achieve high and sustained antigen presentation, with reduced heterogeneity within the cell population and they express CD40L in order to enhance MHC-class I and class II by the APC. In some embodiments, the antigen is a tandem-minigene. In some embodiments, the antigen is stably expressed. In some embodiments, the tandem-minigene encodes one or more mutated protein sequence found within a tumor. In some embodiments, the APC is used to identify and/or characterize TCRs for therapy of humans, including cancer therapy.

Some embodiments of the APC described herein are suitable for therapeutic TCR discovery efforts that are performed on an individual, fully personalized basis or involve large numbers of different MHC-class I and II alleles (or HLA-alleles). In those therapeutic TCR discovery efforts:
(1) TCR discovery is carried out against all class I and II HLA-alleles of the individual and cover one or all HLA-alleles of a given individual by use of an APC derived from an autologous cell. Thus, TCR discovery is independent of the HLA-allele of the individual.
(2) Only TCRs specific for antigens that can be processed and presented by autologous cells are identified by the screening. Thus, TCRs should recognize antigens that are truly processed and presented by cells, and processing and presentation of candidate antigens during TCR discovery can be evaluated; and
(3) Reporter T cells—rather than patient-derived T cells—as well as APC populations preferably with homogenously and stable levels are used in a functional genetic screening, reducing or eliminating heterogenous functional phenotypes with regards to T cells and APCs which would negatively impact screening sensitivity. In some embodiments, APCs expressing the same TMG should roughly express the same antigen levels.

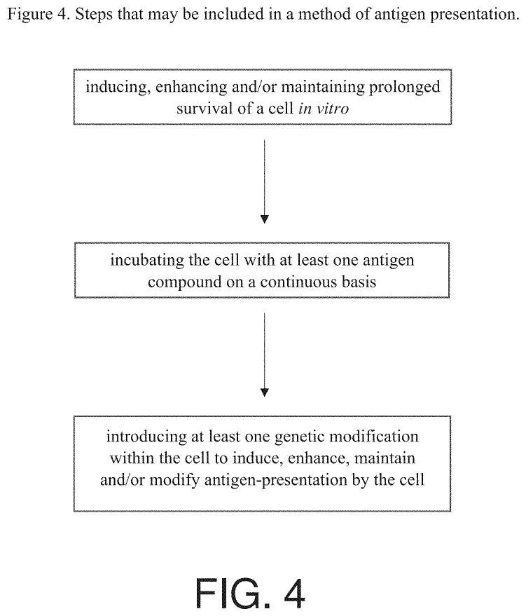

Some embodiments relate to a method of antigen presentation. As shown in FIG. 4, a method can comprise one or more of the following steps: a) inducing, enhancing and/or maintaining prolonged survival of a cell in vitro; b) incubating the cell with at least one antigen compound on a continuous basis; and c) introducing at least one genetic modification within the cell to induce, enhance, maintain and/or modify antigen-presentation by the cell. Additional steps can be added and can be intervening. In some embodiments, any one or more of the above steps can be repeated.

In some embodiments, the inducing, enhancing and/or maintaining prolonged survival of the cell comprises co-culturing the cell with CD40L-expressing feeder cells. Some embodiments further comprise co-culturing the cell with IL-2, IL-4 and/or IL-21. In some embodiments, the combination of IL-2, IL-4 and IL-21 can lead to T cell outgrowth. In some embodiments, IL-2 can be used alone to activate B-cells, then after about 1-3 days, for example 48 hours, one can use the combination of these 3 cytokines for 24 hours before one cultures the B cells just with IL-21 of the three ingredients. In some embodiments, hCD40L-feeders+IL2/IL4/IL21 are used for short-term survival as T cells grow out.

In some embodiments, the inducing, enhancing and/or maintaining prolonged survival of the cell comprises expressing BCL-6 and BCL2-like 1 genes in the cell. Some embodiments further comprise expressing CD40L gene in the cell.

In some embodiments, the inducing, enhancing and/or maintaining prolonged survival of the cell comprises infecting the cell with EBV. Some embodiments further comprise expressing CD40L gene in the cell.

In some embodiments, the BCL-6, BCL2-like 1, or CD40L gene is introduced into the cell through any method known to those of skill in the art, including, for example, retroviral transduction, site-specific integration, transposons, CRISPR/Cas9, or TALEN. In some embodiments, the gene can be introduced via viral transduction, electroporation, transfection, site-specific integration, transposons, CRISPR/Cas9, or TALEN.

In some embodiments, the prolonged survival of the cell is at least three months in cell culture in vitro. In some embodiments, the prolonged survival of the cell is 0.5. 1, 2, 3, 4, 5, 6, 7, 8, 9, 10, 11, 12, 13, 14, 15, 16, 17, 18, 19, 20, 21, 22, 23, or 24 months in cell culture in vitro.

In some embodiments, the at least one antigen compound is encoded by one transgene encoding between one and forty polypeptides. In some embodiments, the transgene encodes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 polypeptides, where the polypeptides can be the same or different. In some embodiments, each polypeptide comprises at least eight or nine amino acids. In some embodiments, each polypeptide comprises 9, 10, 11, 12, 13, 14, 15, 16, 17, 18, 19, 20, 21, 22, 23, 24, 25, 26, 27, 28, 29, 30, 50, 75, 100, 200, 300, 400, 500, 600, 700, 800, 900, 1000 or more amino acids.

In some embodiments, at least one antigen compound is stably expressed in the cell. The antigen encoding transgene can be delivered by any methods that lead to stable transgene integration, including viral gene delivery systems (retro or lentivirus) as well as non-viral gene delivery methods such as site-specific integration, CRISPR/Cas9, TALEN, and Transposon-based vectors. In some embodiments, the transgene can be added via viral transduction, electroporation, squeezing, transfection, site-specific integration, transposons, CRISPR/Cas9, or TALEN.

In some embodiments, at least one antigen compound is transiently expressed in the cell.

Antigen-presentation by APCs can be enhanced by various strategies. First, the co-incubation with adjuvants, such as Toll-like receptor (TLR) ligands, cytokines and soluble receptor ligands (e.g. trimerized CD40L), has been shown to enhance antigen-presentation by APCs. Second, genetic engineering can be used to activate or inactivate certain antigen-presentation pathways within APCs. For example, the modulation of Class II Transcription Activator (CIITA) can be used to control MHC-class II presentation in APCs while the genetic knock-out of Beta-2-Microglobulin (B2M) or Transporter Associated with antigen Presentation (TAP) will abolish MHC-class I antigen presentation. In some embodiments, genetic engineering includes expression of CD40L by the cell. In some embodiments, CD40L enhances antigen-presentation and induces, enhances and maintains prolonged survival of the APC. In some embodiments, engineering includes expression of co-stimulatory molecules, including but not limited to CD80, CD83 and CD86. In some embodiments, genetic engineering includes genetic knockout of one or more inhibitory receptor molecules, including but not limited to, PD-L1. In some embodiments genetic engineering includes the expression of soluble factors, such as cytokines and chemokines, including but not limited IL-2. In some embodiments, genetic engineering includes modifying cells to express or deplete components of the antigen presenting machinery. In some embodiments, genetic engineering includes modifying cells to express components of the antigen presenting machinery that are modified to enhance their function in antigen presentation. In some embodiments, genetic engineering includes modifying cells to express factors other than CD40L that enhance class I and/or class II presentation. In some embodiments, genetic engineering includes modifying cells to display enhanced trafficking of polypeptides encoded by a TMG to the cell membrane and/or to endocytic vesicles. In some embodiments, genetic engineering includes modifying cells to induce random gene expression, for instance by expression of AIRE. Third, antigen-presentation by APCs can be enhanced by delivering the antigen compound in an optimized formulation. In some embodiments, the antigen compound is stably expressed from a tandem-minigene that is designed to target both MHC-class I and II antigen presentation pathways by inclusion of specific sequences, including but not limited to, LAMP-1 signaling and transmembrane sequences. In some embodiments, the transgene contains other sequences that allow efficient targeting of the antigen into the MHC-class II presentation pathway, for instance by inducing trafficking to the cell membrane or endocytic vesicles. In some embodiments, the various regions encoding minigenes are separated by spacer sequence. In some embodiments, other sequences are added to the transgene to increase stability of the encoded polypeptide. In some embodiment, other sequences are included in the transgene to increase the efficiency of MHC-class I or II related antigen presentation. In some embodiments, the tandem minigene includes at least one marker to detect and enrich APCs expressing the TMG in order to ensure high and homogenous antigen. In some embodiments, antigen-presentation by APCs is enhanced by incubation with a small-molecule, antibody or nucleic acid, including but not limited to siRNA, shRNA and miRNA.

In some embodiments, the antigen compound is a polypeptide with at least 12 amino acids that is electroporated into the APC in order to target both MHC-class I and II antigen presentation pathways. In some embodiments, the polypeptide includes one or more non-natural amino acids. In some embodiments, the polypeptide is chemically modified to alter its properties, including but not limited to stability, protease sensitivity and binding to MHC-molecules. In some embodiments, electroporation is substituted with an alternative method that allows a polypeptide to cross the cell membrane. In some embodiments, titrating amounts of the polypeptide are electroporated into the APCs in order to optimize antigen-presentation in relation to antigen amounts. This allows to optimize antigen-presentation for TCR characterization studies. In some embodiments, electroporation with pools of polypeptides is useful for TCR discovery, including but not limited to, the screening of TCR libraries. In some embodiments, agents enhancing the processing and/or presentation of the class I or II antigen machinery are co-delivered with the polypeptide.

In some embodiments, the antigen compound is a polypeptide with at least 8 amino acids that is electroporated into the APC. In some embodiments, the antigen compound is a polypeptide of 8, 9, 10, 11, 12, 13, 14, 15, 16, 17, 18, 19, 20, 21, 22, 23, 24, or 25 amino acids that is electroporated into the APC. Persons of skill in the art will recognize that different lengths of polypeptides can be used or compared in some embodiments of the invention or to determine different features or properties of the TCR, antigen, or peptide-MHC complex, including identification of the minimal epitope. In some embodiments, the antigen compound that is incubated with the cell is processed by the cell before an antigen is presented.

As described herein, an antigen compound typically refers to the molecule or unique aspect thereof (such as the bound peptide in a peptide-MHC complex) recognized by an immunological protein (e.g., a TCR). Persons of skill in the art will recognize that in some contexts, a precursor molecule (such as a 25-mer polypeptide) may be referred to as an antigen compound even though the molecule recognized and bound by the immunological protein is a molecule (e.g., a polypeptide) that is modified, processed, or complexed from the precursor. Persons of skill in the art will also recognize that in some contexts, a polynucleotide encoding a polypeptide may be referred to as an antigen compound even though the molecule or unique aspect thereof that is recognized and bound by the immunological protein is a molecule (e.g., a polypeptide) encoded by or expressed from the polynucleotide; in such cases, the antigen compound is understood to be a polypeptide expressed from the polynucleotide and not the polynucleotide itself. Persons of skill in the art will also recognize that immunological proteins can bind polynucleotides or small molecules as antigens; and that in such cases, an antigen compound need not be a polypeptide. This shorthand is used to avoid having to restate the same idea in various formats. As used herein, an antigen compound is “incubated” with a cell (e.g., an APC), when the antigen compound is exogenously introduced in or presented to the cell by any method including, but not limited to, addition of the antigen compound to the cell culture media, electroporation of the antigen compound into the cell, or expression of the antigen compound from an exogenous genetic modification. A “TCR antigen compound” specifically refers to the peptide or other molecule that is bound by an MHC (or MHC-like molecule), or to a precursor peptide to the peptide bound by an MHC (or MHC-like molecule), and not to the peptide-MHC complex as a whole.

In the above-described embodiments, several elements to create an APC for TCR discovery can be combined:

In some embodiments, autologous primary human B cells are used to allow TCR discovery against all HLA-alleles of a given individual. Immortalization of B cells either based on infection by Epstein-Barr-Virus (EBV) or stable transduction with BCL-6 and BCL-XL allows one to obtain high cell numbers. The BCL-6/BCL-XL immortalization method are enhanced by demonstrating that CD40 receptor stimulation on B cells—a useful stimulation to maintain primary human B cells in vitro—can be achieved by direct transduction of B cells with a CD40L molecule rather than by co-culture of B cells with CD40L-transduced feeder cells. This modification significantly improves the industrial application by easing the maintenance of the immortalized B cells in cell culture. In some embodiments, autologous primary human B cells for TCR discovery can be generated from small volumes of human blood. In some embodiments, the volume of human blood used includes 1, 2, 3, 4, 5, 6, 7, 8, 9, 10, 11, 12, 13, 14, 15, 16, 17, 18, 19 or 20 ml of human blood. In some embodiments, autologous primary human B cells for TCR discovery are obtained from other human tissue, including but not limited to, tumor and lymph node biopsies. In some embodiments, autologous primary human B cells for TCR discovery are expanded to 10, 20, 30, 40, 50, 60, 70, 80, 90, 100, 150, 200, 250, 500 and 750 Million or 1, 2, 3, 4, 5, 6, 7, 8, 9 and 10 Billion cells.

Second, in some embodiments, the antigen is delivered by stable transduction of immortalized B cells. In some embodiments, stable transduction is achieved by integration of a nucleic acid sequence into the genome of the cells. In some embodiments, stable transduction leads to continuous expression of the antigen. This mode of delivery allows one to assess antigen processing and presentation. Furthermore, continuous expression of saturating amounts of antigen minimizes heterogenous antigen expression within the APC population (which would negatively impact sensitivity of TCR discovery). In addition, stable expression also eases the generation of high numbers of APCs. In some embodiments, delivery of antigen in a transient form may be preferable for certain applications, included but not limited to, TCR isolation and TCR characterization studies.

Third, in some embodiments, MHC-class I and MHC-class II antigen presentation of the B cells is modulated by expression of a CD40L transgene. T cell activation of MHC-class I and MHC-class II restricted T cells is enhanced in the presence of CD40L-modified B cells, thereby increasing TCR discovery sensitivity.

The embodiments described herein can be applied to TCR discovery in various therapeutic areas besides oncology, such as autoimmunity and infectious disease. Some embodiments relate to the identification of TCR sequences with defined functional properties (e.g. recognition of specific antigen) for therapeutic use, e.g. in cancer, from a large collection of TCR variants. Some embodiments relate to the characterization of TCR sequences (e.g. confirmation of TCR specificity, mapping of HLA-restriction or evaluation of TCR sensitivity) for therapeutic use, e.g. in cancer.

Furthermore, the invention may be useful to expand patient-derived, antigen-specific T cells for therapeutic use. Examples include the expansion of tumor-infiltrating lymphocytes (TIL) by co-culture with neo-antigen presenting B cells, e.g. to replace dendritic cells as APCs, or the expansion of blood-derived T cells by co-culture with neo-antigen presenting B cells. These approaches depend on the use of autologous antigen presenting cells for T cell activation to enable their expansion.

In some embodiments, besides peripheral blood, primary human B cells can be derived from other human tissues that contain B cells, including but not limited to, tumor tissue, lymph nodes, spleen, cord blood and bone marrow.

In some embodiments, primary human B cells may be obtained by differentiation of precursor cells, including but not limited to hematopoietic stem cells.

In some embodiments, human B cells may be obtained by differentiation of induced pluripotent stem cells (iPSC).

The immortalization of B cells with EBV (Traggiai et al. Methods Mol Biol 2012) and BCL-6/BCL-XL (Kwakkenbos et al. Nat Med 2008) has been described previously. The various embodiments provided herein is the first demonstration that concomitant transduction with CD40L is a feasible strategy to supply CD40 receptor stimulation on B cells and can replace co-culture with CD40L expressing feeder cells (Kwakkenbos et al. Nat Med 2008) and/or stimulation by trimerized CD40L recombinant protein (Lapointe et al. Cancer Res 2003). The presented solution is preferable for industrial application as it eliminates the need to supplement any other exogenous CD40L stimulation (e.g. by CD40L expressing feeder cells) and furthermore enhances MHC-class I and II presentation by the B cells

Stable expression of the antigen as achieved by retroviral transduction offers several advantages: based on stable antigen-expression for prolonged time periods, it is possible to initially modify a small number of B cells and subsequently expand the cell population to sufficient cell numbers rather than loading larger number of APCs with antigen. The latter approach is technically more cumbersome (i.e. more cells have to be loaded, more antigen is required), thereby hampering industrial application. More importantly, it will also lead to heterogeneous antigen expression levels within the APC population which can negatively impact the sensitivity of TCR discovery. Of note, stable expression of an antigen-encoding transgene additionally facilitates detection and selection of antigen-expressing B cells which is either impossible (peptides and protein) or challenging (mRNA, DNA-plasmid) based on other antigen compounds.

In some embodiments, TCR discovery can identify T cell receptor genes with specificity for mutated proteins presented in tumor cells. The vast majority of neo-antigens are formed by non-synonymous point mutations leading to single amino acid changes within a protein sequence. Mutated protein sequences are encoded as 25-amino acid long polypeptides (with 12 amino acids up- and downstream the mutated amino acid residue) in minigenes. Twelve minigenes are combined into a tandem-minigene (TMG) construct. In some embodiments, antigens in the form of a TMG can be delivered. A person of skill in the art will recognize that numbers of minigenes other than twelve can be used in the TMG format, including 2, 3, 4, 5, 6, 7, 8, 9, 10, 11, 13, 14, 15, 16, 17, 18, 19, 20, 21, 22, 23, 24, 25, 26, 27, 28, 29, 30, 31, 32, 33, 34, 35, 36, 37, 38, 39, 40, or more than 40 minigenes. In some embodiments, TCR discovery may focus on specific types on neo-antigens, including but not limited to, alternative open reading frames or neo-antigens shared between patients (e.g. mutations in KRAS, p53, PI3K and other driver mutations). In some embodiments, TCR discovery may focus on viral antigens occurring in cancer, including but not limited to, Human Papillioma Virus (HPV), Merkel Cell and Epstein-Barr Virus (EBV). In some embodiments, TCR discovery may focus on human endogenous retroviruses (HERVs). In some embodiments, TCR discovery may focus on shared tumor antigens. In some embodiments, T cells or TCRs discovered with the process provided herein are used for cell therapy. In some embodiments, the cell therapy is based autologous patient donor cells. In some embodiments, the cell therapy is based on allogeneic donor cells. In some embodiments, the allogeneic donor cells are derived from a healthy human donor. In some embodiments, the allogeneic donor cells are derived from a hematopoietic, pluripotent, induced or another stem cell. In some embodiments, the allogeneic donor cells are genetically engineered to improve their persistence or function. In some embodiments, the cell therapy is a TCR-engineered cell therapy.

In some embodiments, stable antigen expression can also be achieved in EBV-immortalized B cells.

Instead of TMGs, also any other open-reading frame designed to be operable for expression in B cells can be used. Examples include single minigenes (as described above), polypeptides of any length and full length protein.

The antigen encoding transgene can be delivered by other methods that lead to stable transgene integration, including other viral gene delivery systems (Lentivirus) as well as non-viral gene delivery methods such as CRISPR/Cas9, TALEN and Transposon-based vectors. In some embodiments, the transgene can be added via viral transduction, electroporation, squeezing, transfection, site-specific integration, transposons, CRISPR/Cas9, or TALEN.

The antigen may be site-specifically integrated into an endogenous locus and under control of an endogenous promotor to achieve defined antigen expression levels.

The antigen may be expressed under an inducible promotor (e.g. a Tetracyclin-regulated promotor) in order to control antigen expression levels.

In the below is the first report demonstrating the stable transduction of primary human B cells with TMG constructs and the first report showing stable transduction of antigen in BCL-6/BCL-XL immortalized B cells. In some embodiments, TMGs are not delivered by either electroporation, viral transduction, transposon, nuclease mediated DNA integration or transfection of plasmid- or linear DNA into APCs, but some other process. In some embodiments, TMGs are delivered by electroporation of or transfection of plasmid-DNA or mRNA into APCs. In some embodiments, TMGs are expressed under the control of an endogenous promotor.

In some embodiments, CD40L transgenes can be used for the expression of CD40L. In some embodiments, there are alternative embodiments beyond CD40L transgene expression. These include: Genetic Knock-out of B2M and/or TAP may be used to abrogate MHC-class I antigen presentation by the engineered B cells; Genetic Knock-out of CIITA and/or CD74 (HLA-DR-antigens-associated invariant chain) may be used to abrogate MHC-class II antigen presentation by the engineered B cells; Genetic Knock-out of specific HLA-alleles may be used to direct TCR discovery towards certain HLA-alleles; and co-stimulatory molecules, including but not limited to, CD80, CD86, CD70, PD-L1 may either be knocked-out or overexpressed to control the threshold for T cell activation by the B cell.

Any desired genetic knock-out as outlined above may be achieved by site-specific integration of any transgene used to engineer the B cell (BCL-6; BCL-XL; antigen; CD40L) into the target locus provided that bi-allelic editing is highly efficient for the chosen target locus.

### Peptide Arrangements

In some embodiments, rather than nucleic acids being delivered to a cell, a peptide can instead (or in addition) be delivered. In some embodiments, provided herein are methods for efficient MHC class I and MHC class II antigen presentation by immortalized B cells via the electroporation of polypeptides of T cell epitopes. In some embodiments, instead of adding the antigen by adding a nucleic acid to a cell, the antigen can be added in polypeptide form via electroporation. The peptide can be part of a T cell epitope.

In some embodiments, a Lonza Nucleofactor 4D electroporation device is used for electroporation. In some embodiments, program DN100 on the device is used.

In some embodiments, the present method involves long (e.g., 14-25) polypeptides of a uniform length (e.g. 25mer) as part of minimal CD8+ T cell epitopes or CD4+ T cell epitopes to be delivered to immortalized B cells, presented by MHC class I and MHC class II molecules, respectively. In some embodiments, presentation by MHC-class I and II molecules on cells can be measured by probing cells with antigen-specific T cells If a T cell epitope of interest is presented by cells in quantities sufficient to activate the antigen-specific T cells, T cell activation markers can be evaluated and will be increased compared to control conditions. In some embodiments, T cell activation markers include, but are not limited to, CD69, CD62L, CD137, IL-2, TNF-a, IFN-g, IL-10, IL17 and GM-CSF. In some embodiments, presentation by MHC-class I and II molecules on cells can be measured by mass spectrometry. In some embodiments, the size of the antigen is long enough to be bound to both MHC-class I and II molecules after appropriate processing. Class I peptides are most commonly 9-11 mers; Class II peptides are 14-25 mers. In some embodiments, these can be longer than 12mers, e.g., 13, 14, 15, 16, 17, 18, 19, 20, 21, 22, 23, 24, 25, 26, 27, 28, 29, or 30mer of longer. In some embodiments, class I peptides are traditionally 9-11 and class II are 14-25. As provided here, longer sequences are used for the present system (e.g., 12 or longer).

In some embodiments, a method for the delivery of polypeptides encoding putative T cell epitopes to immortalized B cells for efficient presentation via the MHC class I or MHC class II antigen presentation pathway for utilization in T cell epitope and T cell receptor (TCR) discovery and characterization studies (hereafter: TCR discovery) is provided.

In some embodiments, the technology can be applied in the identification of neo-antigen specific TCR genes from non-viable tumor biopsies on a per patient basis. DNA and RNA derived from the patient's tumour biopsy can be used for targeted genomic sequencing to obtain both bulk TCR and mutanome sequence information. Some embodiments include generating TCR libraries by combinatorial assembly of tumor-derived TCRα and TCR chains identified by the bulk TCR chain sequencing of DNA or RNA isolated from the tumor tissue. TCRαβ pairs are encoded as transgenes of approx. 1.8 kb and introduced into reporter T cells. In parallel, a small peripheral blood sample (e.g. 10 mL) is also taken from the patient and used to generate immortalised B-cells. The tumour mutanome sequence information is used to synthesise a library of neo-antigen genes and these are introduced into the immortalized B-cells, thereby generating personalized neo-antigen presenting B-cells. By stimulation with antigen-expressing cells, reporter T cells expressing antigen-reactive TCRαβ combinations can be selected in a genetic variant library screening based on expression of T cell activation markers, including but not limited to CD69 and CD62L. Given the combinatorial assembly, each TCR variant can only be unambiguously identified by determining both TCRα and TCRβ variable sequences. Hence, transgenes encoded in reporter cells isolated during the genetic screen are recovered as PCR amplicons of approx. 1.5 kb, sequenced in full length using Oxford Nanopore sequencing (or any other appropriate sequencing approach) and analyzed using bioinformatics. This process identifies neo-antigen specific TCR leads that can be further evaluated for potential use in cancer therapy. In some embodiments, the APCs presented herein can be used to screen non-combinatorial libraries.

In some embodiments, the further evaluation can involve a process of TCR and antigen characterization to ensure safety and to optimize the therapeutic effects of the therapy. Laboratory based immuno-assays, involving the co-culture of T cells engineered to express identified neo-antigen specific TCR leads and antigen expressing autologous immortalized B cells, are conducted to obtain information on, optionally, one or more of the following: 1) Mutation specificity—demonstrated by specific recognition of the mutated antigen, 2) HLA restriction—MHC class I/II restriction is determined, 3) TCR sensitivity—evaluated by peptide titration and/or recognition of targets cells with expression of defined antigen levels, 4) Absence of off-target activity—demonstrated by absence of reactivity against autologous B cells and autologous B cells engineered to express wild-type (non-mutated) antigen.

For the purposes of TCR and antigen characterization in a fully personalized neo-antigen specific TCR therapy setting, a variety of different antigen formats and types of antigen delivery systems can be utilized. The delivery of antigen to APCs in the format of a polypeptide is useful as polypeptides can be produced in a high throughput manner and are well suited to industrial application. Co-incubation of APCs with a polypeptide of variable length leads to antigen-presentation by APCs. Depending on the length of the peptide, presentation preferably occurs by the MHC-class I (approx. 9-11 amino acids) or MHC-class II (approx. 14-25 amino acids) presentation pathway. Notably, shorter (9-11 aa) peptides typically do not bind to MHC-class II molecules and longer peptides (14+ amino acids) are not always reliably processed and presented by the MHC-class I presentation pathway. Therefore, co-incubation of APCs with polypeptides of a uniform length (e.g. 25mer) have not traditionally been reliably used for the purpose of evaluating antigen recognition by both MHC-class I restricted TCRs and MHC-class II restricted TCRs.

In some embodiments, the embodiments provided herein can be applied to activate, expand and/or characterize TCRs specific for mutated proteins in tumor suppressors and oncogenes, including but not limited to KRAS, p53 and PI3K. Polypeptides containing common single amino acid changes are electroporated into APCs to express antigen through both MHC-class I and II pathways. Antigen-expressing APCs are used to activate, expand and/or characterize mutation specific T cells or TCRs. In some embodiments, T cells are derived from tumor lesions. In some embodiments, T cells are derived from peripheral blood. In some embodiments, T cells express TCRs obtained from a TCR library. In some embodiments, the technology can be used to activate, expand and/or characterize TCRs restricted to either MHC-class I and II or a mix of TCRs restricted to both. In some embodiments, the method is an antigen delivery method to APCs applying polypeptides of a uniform length which results in efficient processing and presentation of putative T cell peptide epitopes via the MHC class I and MHC class II pathway, thereby allowing characterization of both MHC class I restricted TCRs and MHC class II restricted TCRs. This can be applied to the characterization of neo-antigen-specific TCRs identified in functional genetic screens of TCR libraries. In some embodiments, the length of the peptide can be: more than a 10 mer in length, 10-14, 14-25, or longer than 25 mer.

Prior to the present disclosure, it had been not been established that (i) longer peptides would be properly processed and presented on MHC-class I after electroporation and (ii) that longer peptides would be presented on MHC-class II after electroporation (normally they are exogenously loaded and if expressed within the cell require specific target sequences (see LAMP-1 sequences used in TMG).

In some embodiments, the amount of the peptide applied can be any functional amount, including, 20 ug/mL-1 pg/mL, for example or 100 uM-1 nM. In some embodiments, the amount can be 0.25 ng/mL or more for MHC class I. In some embodiments, the amount can be 10 ng/mL for MHC II, e.g., 50, 100, 150, 160, 200, ng/mL or more of the peptide.

In prior approaches, if one did not know the exact restriction element, one would have to test both a series of short peptides (for Class I; a series because the exact 9-11mer may not be known, so overlapping peptides would be made) and longer peptides (for Class II). However, with some of the embodiments presented herein, it is possible to use a single 25mer and cover both Class I and Class II. Thus, in some embodiments, a single antigen sequence can be used. This can be applied to any of the embodiments provided herein.

In some embodiments, the 25mer can be longer or shorter than 25 if there is a frameshift mutation (e.g., 13 to 300 amino acids). In some embodiments, a cell optimized to present antigen to T cells is provided. The optimization can include the delivery of polypeptides of a uniform length that are or include putative T cell antigen(s) via electroporation allowing for the efficient processing and presentation of putative T cell peptide epitopes derived from the polypeptide via the MHC class I or MHC class II antigen processing and presentation pathway. In some embodiments (e.g., a frameshift mutation), multiple overlapping peptides of a uniform length (e.g., 25 amino acids) can be used for electroporation to determine the antigen.

In some embodiments, a method to deliver polypeptides of a uniform length to immortalized primary human B cells is provided. The polypeptides can be delivered via electroporation allowing for efficient processing and presentation of putative T cell peptide epitopes derived from the polypeptide via the MHC class I or MHC class II antigen processing and presentation pathway, thereby allowing for the characterization of MHC class I restricted TCRs and MHC class II restricted TCRs. In some embodiments, a method to deliver polypeptides of a uniform length to other human professional antigen presenting cells, including but not limited to dendritic cells, monocytes and macrophages is provided. In some embodiments, a method to deliver polypeptides of a uniform length to tumor cells or cell lines is provided.

In some embodiments, the process can be used for characterizing neo-antigen specific TCRs identified as part of a neo-antigen TCR isolation process from tumor biopsies. For example, this can include, in a therapeutic application to develop fully personalized neo-antigen specific engineered TCR therapy for the treatment of solid cancer. This can occur in a process utilizing functional genetic screens of tumor-derived TCR libraries that allows the identification of neo-antigen specific TCR genes from tumor biopsies on an individual patient basis. Following their identification, neo-antigen specific TCR leads can be further evaluated for potential use in cancer therapy.

In some embodiments, the evaluation involves a process of TCR and antigen characterization to ensure safety and to optimize the therapeutic effects of the therapy. Laboratory based immuno-assays, involving the co-culture of T cells engineered to express identified neo-antigen specific TCR leads and antigen expressing autologous immortalized B cells, can be conducted to obtain information on one or more of the following: 1) Mutation specificity—demonstrated by specific recognition of the mutated antigen, 2) HLA restriction—MHC class I/II restriction is determined, 3) TCR sensitivity—evaluated by peptide titration and/or recognition of targets cells with expression of defined antigen levels, 4) Absence of off-target activity—demonstrated by absence of reactivity against autologous B cells and autologous B cells engineered to express wild-type (non-mutated) antigen.

In some embodiments, antigen is delivered into immortalized B cells (e.g. cells are immortalized by BCL-6/BCL-XL overexpression via retroviral gene transfer). In some embodiments, immortalized B cells are electroporated in the presence of a polypeptide of a uniform length (e.g. 25mer), which results in efficient processing and presentation of putative T cell peptide epitopes encoded by the polypeptide via the MHC class I and MHC class II antigen processing and presentation pathway. This allows for the characterization of both MHC class I restricted TCR leads and MHC class II restricted TCR leads in subsequent immuno-assays involving the co-culture of T cells engineered to express identified neo-antigen specific TCR leads and polypeptide antigen electroporated autologous immortalized B cells.

In some embodiments, the process can be used for characterizing TCRs isolated from peripheral blood. For example, this can include, in a therapeutic application to develop fully personalized neo-antigen specific engineered TCR therapy for the treatment of solid cancer. This can occur in a process utilizing functional genetic screens of blood-derived TCR libraries that allows the identification of neo-antigen specific TCR genes from patient blood on an individual patient basis. Following their identification, neo-antigen specific TCR leads can be further evaluated for potential use in cancer therapy.

In some embodiments, the process can be used for characterizing TCRs isolated from a synthetic TCR library. For example, this can include synthesizing a TCR library and screening this library for TCRs with a desired antigen-specificity. In some embodiments, a desired TCR is specific for a tumor neo-antigen. In some embodiments, a desired TCR is specific for a viral antigen. In some embodiments, a desired TCR is specific for a shared tumor-antigen. Following their identification, TCR leads can be further evaluated for potential use in cancer therapy.

In some embodiments, delivery of antigen in the form of a polypeptide of uniform length via electroporation offers one or more advantages. Firstly, polypeptides can be produced rapidly in a high throughput manner and are well suited to industrial application. Secondly, in contrast to co-incubation as a means of polypeptide antigen delivery to APCs, electroporation allows for a polypeptide of a uniform length (e.g. 25mer) to be used to evaluate antigen recognition by both MHC class I restricted TCRs and MHC class II restricted TCRs. Thirdly, the efficient processing and presentation via the MHC class I and MHC class II pathways of putative T cell epitopes that is achieved with electroporation of APCs with graded concentrations of polypeptide of a uniform length allows for the assessment of the sensitivity of TCR leads without the need to first define the class restriction and the minimal T cell epitope in immuno-assays, and therefore is well suited to the personalised nature of the TCR therapy and the short timelines often involved to complete the TCR and antigen characterization process. Fourth, the efficient processing and presentation via the MHC class I and MHC class II pathways of putative T cell epitopes that is achieved with electroporation enables rapid evaluation of large numbers of peptides. In some embodiments, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000 and 10000 peptides are evaluated. In some embodiments, a complete neo-antigen set is evaluated. In some embodiments 100% or less of the antigen se is evaluated, e.g., 99.9, 99, 98, 97, 96, 95, 90, 80, 70, 60, 50% or less of a full neo-antigen set is evaluated.

In some embodiments, the efficient processing and presentation via MHC class I and MHC class II of putative T cell epitopes via electroporation of a polypeptide of a uniform length can also be achieved in EBV-immortalized B cells.

In some embodiments, instead of B cells as an APC, other types of APCs such as dendritic cells, macrophages or artificial APCs (e.g. K562 cells engineered to express relevant HLA alleles) can be utilized.

In some embodiments, using crude peptide for electroporation to the APC has one or more of the following advantages: 1) it can be cheaper; 2) crude peptide will not be purified, so it will naturally contain length variants, e.g., if a 25mer is synthesized there will be 9, 10, 11mer or more peptides in the mix. This will enhance presentation because some of these shorter peptides may contain the right T cell epitope and bind to MHC directly without any need for processing. In some embodiments, efficient processing and presentation on MHC class I and MHC class II of putative T cell epitopes by APCs (e.g. immortalized B cells, DCs) via electroporation of polypeptides of a non-uniform length (e.g. crude peptide extracts) can be used.

In some embodiments, the process involves the delivery via electroporation of polypeptides of a uniform length to APCs (e.g., primary human immortalized B cells) to be used as a means to achieve highly efficient processing and presentation of putative T cell peptide epitopes encoded by the polypeptide via the MHC class I and MHC class II antigen processing and presentation pathways.

In some embodiments, the process allows for the characterization of both MHC class I restricted TCRs and MHC class II restricted TCRs using a single antigen format and antigen delivery method to APCs.

In some embodiments, the process comprises the delivery via electroporation of polypeptides of a uniform length to APCs (e.g., primary human immortalized B cells) as a means to achieve highly efficient processing and presentation of putative T cell peptide epitopes encoded by the polypeptide via the MHC class I and MHC class II antigen processing and presentation pathways. When applied in the context of TCR characterization this approach offers, in some embodiments, one or more of the following advantages: 1) It allows for a polypeptide of a uniform length (e.g. 25mer) to be used to evaluate antigen recognition by one or both MHC class I restricted TCRs and MHC class II restricted TCRs without knowledge of class restriction of the TCRs. In some embodiments, if one does not know which applies, then both option applies for example, if a set of TCRs specific for the same antigen is tested and some are Class I and some are Class II. 2) It allows for the efficient processing and presentation via the MHC class I and MHC class II pathways of putative T cell epitopes that is achieved with electroporation of APCs with graded concentrations of polypeptide of a uniform length allows for the assessment of the sensitivity of TCR leads without the need to first define the minimal T cell epitope in immuno-assays. In some embodiments, the graded concentration can be 1 uM->100 pM with a 10 step dilution for the titration. In some embodiments, one can see a signal from 10 nM (class I) or 100 nM (class II). In some embodiments, this is further superior in Bcl-6/xL B cells. 3) Polypeptides can be produced rapidly in a high throughput manner and are well suited to industrial application.

In some embodiments, the process can be part of a high-throughput TCR discovery on a per patient basis with a variety of platforms. Following identification of TCR leads they are further evaluated in a process of TCR and antigen characterization to ensure safety and to optimize the therapeutic effects of the therapy. Laboratory based immuno-assays, involving the co-culture of T cells engineered to express identified neo-antigen specific TCR leads and antigen expressing autologous immortalized B cells, can be conducted to obtain information on one or more the following: 1) Mutation specificity—demonstrated by specific recognition of the mutated antigen, 2) HLA restriction—MHC class I/II restriction is determined, 3) TCR sensitivity—evaluated by peptide titration and/or recognition of targets cells with expression of defined antigen levels, 3) Absence of off-target activity—demonstrated by absence of reactivity against autologous B cells and autologous B cells engineered to express wild-type (non-mutated) antigen.

In some embodiments, the process allows one to use a single antigen format, polypeptide of uniform length (e.g. 25mer), combined with a single antigen delivery approach, electroporation, to generate antigen expressing autologous B cells that can be used for all the TCR and antigen characterizations assays that are required to select safe and effective TCRs for a fully personalized TCR therapy platform. In some embodiments, it is particularly well-suited to the personalised nature of the TCR therapy and the short timelines required to complete the TCR and antigen characterization process.

In contrast, current combinations of antigen format & antigen delivery technologies that are used to characterize T cells/TCRs can be more time consuming as a single combination of antigen format and antigen delivery system cannot be used to deliver all the TCR/antigen characterization required to select safe and effective TCRs for fully personalized TCR therapy. In some embodiments, for the peptide embodiments provided herein, the process can include a 1 day co-culture, and a 1 day readout. In some embodiments, for the DNA constructs provided herein, the process can include a 1 week transfection+transduction+4 days selection+expansion of cells+1 day co-culture+1 day readout).

In some embodiments, the process involves the characterization of TCR sequences (e.g. confirmation of TCR specificity, mapping of HLA-restriction or evaluation of TCR sensitivity) for therapeutic use, e.g. in cancer. In some embodiments, the process involves the identification of TCR sequences with defined functional properties (e.g. recognition of specific antigen) for therapeutic use, e.g. in cancer, from a large collection of TCR variants. In some embodiments, the process is particularly suited for fully personalized TCR discovery that aims to utilize both HLA class I and HLA class II restricted TCRs. Given that it allows for TCR/antigen characterization for both HLA class I and HLA class II restricted TCRs with a single antigen format and antigen delivery system it is particularly well-suited to personalised/customized TCR therapy. In some embodiments, it allows for especially short timelines for completing the TCR and antigen characterization process. For example, instead of taking approx. 10 days for transduction and selection of antigen-expressing APCs, the process can be reduced by as much as 90%, and be done in as little time as 1 day. Since the TCR/antigen characterization process is a valuable step in the TCR discovery process, a streamlined and rapid TCR/antigen characterization process can be useful for TCR discovery and characterization.

In some embodiments, the process can be applied to TCR discovery in all therapeutic, including those areas besides oncology, such as autoimmunity and infectious disease. In some embodiments, the process can be useful to expand patient-derived, antigen-specific T cells for therapeutic use. Examples include the expansion of tumor-infiltrating lymphocytes (TIL) by co-culture with neo-antigen presenting APCs, e.g. dendritic cells, or the expansion of blood-derived T cells by co-culture with neo-antigen presenting APCs. These approaches depend on the use of autologous antigen presenting cells that have been modified to express antigens of interest for T cell activation to allow their expansion.

In some embodiments, any of the presently disclosed APCs or methods involving APCs or other related embodiments can be applied to any of the methods or compositions provided in U.S. Pub. No. 2021/0040558, published Feb. 11, 2021 in the corresponding APC related composition or method or antigen screening, etc. That is, any one or more of the APC related aspects in U.S. Pub. No. 2021/0040558 can employ any of the embodiments provided herein. U.S. Pub. No. 2021/0040558, published Feb. 11, 2021, is hereby incorporated by reference in its entirety, and further including those embodiments involving APCs and antigen presentation.

As used herein, stimulating CD40 receptor on B cells can include CD40L based stimulation which may be referred to as CD40L stimulation.

### Arrangements

In some embodiments, any of the following arrangements or subparts thereof can be part of or combined with the embodiments provided herein. Arrangements are numbered 1-107 as follows:

1. A method of engineering a cell for antigen presentation to T cells, the method comprising:
inducing, enhancing and/or maintaining prolonged survival of a cell in vitro;
incubating the cell with at least one antigen compound on a continuous basis; and
introducing at least one genetic modification within the cell to induce, enhance, maintain and/or modify antigen-presentation by the cell.

2. The method of arrangement 1, wherein the cell comprises a primary human B cell, optionally wherein the primary human B cell is autologous with respect to the T cells or to a TCR presented by the T cells.

3. The method of arrangement 2, wherein the primary human B cell is derived from peripheral blood.

4. The method of arrangement 2, wherein the primary human B cell is derived from a human tissue that contain B cells

5. The method of arrangement 4, wherein the human tissues comprise tumor tissue, lymph nodes, spleen, cord blood, body fluids, and bone marrow.

6. The method of arrangement 2, wherein the primary human B cell is obtained by differentiation of a precursor cell.

7. The method of arrangement 6, wherein the precursor cell comprises hematopoietic stem cells.

8. The method of arrangement 2, wherein the primary human B cell is obtained by differentiation of induced pluripotent stem cells (iPSC).

9. The method of arrangement 1, wherein the inducing, enhancing and/or maintaining prolonged survival of the cell comprises co-culturing the cell with CD40L-expressing feeder cells.

10. The method of arrangement 9, further comprising co-culturing the cell with IL-2, IL-4 and IL-21.

11. The method of arrangement 1 or 10, wherein the inducing, enhancing and/or maintaining prolonged survival of the cell comprises expressing BCL-6 and BCL2-like 1 genes in the cell.

12. The method of arrangement 1, wherein the inducing, enhancing and/or maintaining prolonged survival of the cell comprises infecting the cell with EBV.

13. The method of arrangement 10, 11, or 12, further comprising expressing CD40L gene in the cell.

14. The method of arrangement 1, wherein the inducing, enhancing and/or maintaining prolonged survival of the cell comprises expressing BCL-6, BCL2-like 1, and CD40L genes in the cell.

15. The method of arrangement 14, wherein the BCL-6, BCL2-like 1, and CD40L genes can be on the same expression construct, or different expression construct.

16. The method of arrangement 11, 13, or 14, wherein the genes are introduced into the cell through viral transduction, electroporation, squeezing, transfection, site-specific integration, transposons, CRISPR/Cas9, or TALEN.

17. The method of arrangement 1, wherein the prolonged survival of the cell is at least three months in cell culture in vitro.

18. The method of arrangement 1, wherein the at least one antigen compound is encoded by one transgene encoding between one and forty polypeptides.

19. The method of arrangement 18, wherein each polypeptide comprises at least eight or nine amino acids.

20. The method of arrangement 1, wherein the at least one antigen compound is expressed in the cell.

21. The method of arrangement 1, wherein the at least one antigen compound is transiently expressed in the cell.

22. An engineered cell, wherein the cell:
has been adjusted to induce, enhance and/or maintain its survival in vitro;
has been incubated with at least one antigen compound; and
has at least one genetic modification to induce, enhance, maintain or modify antigen-presentation by the cell.

23. The engineered cell of arrangement 22, wherein adjusted to induce, enhance and/or maintain its survival in vitro comprises expressing BCL-6 and BCL2-like 1 genes in the cell.

24. The engineered cell of arrangement 22, wherein adjusted to induce, enhance and/or maintain its survival in vitro comprises infecting the cell with EBV.

25. The engineered cell of arrangement 23 or 24, further comprising expressing CD40L gene in the cell.

26. The engineered cell of arrangement 23 or 25, wherein the genes are introduced into the cell through viral transduction, electroporation, squeezing, transfection, site-specific integration, transposons, CRISPR/Cas9, or TALEN.

27. The engineered cell of arrangement 22, wherein the cell survival is at least three months in cell culture in vitro.

28. The engineered cell of arrangement 22, wherein the at least one antigen compound is encoded by at least one transgene encoding between one and forty polypeptides.

29. The engineered cell of arrangement 28, wherein each polypeptide comprises at least eight amino acids.

30. The engineered cell of arrangement 22, wherein the at least one antigen compound is stably expressed in the cell.

31. The engineered cell of arrangement 22, wherein the at least one antigen compound is transiently expressed in the cell.

32. An engineered cell comprising:
a nucleotide sequence for expression of a survival factor;
a nucleotide sequence for expression of at least one transgene encoding an antigen; and
a nucleotide sequence for expression of CD40L.

33. The engineered cell of arrangement 32, wherein the survival factor comprises BCL-6 and/or BCL-XL.

34. The engineered cell of arrangement 32, wherein the at least one transgene encoding an antigen encodes for between one and forty polypeptides.

35. The engineered cell of arrangement 34, wherein each polypeptide comprises at least eight amino acids.

36. The engineered cell of arrangement 32, wherein the nucleotide sequence for expression of CD40L comprises a gene that provides stable expression of CD40L.

37. A primary human B cell to present antigen to T cells, the cell comprising:
a nucleotide sequence providing stable expression of BCL-6 and BCL-XL;
a nucleotide sequence providing stable expression of between one and forty polypeptides, each polypeptide encoding at least eight amino acids, and each polypeptide being an antigen; and
a nucleotide sequence providing stable expression of CD40L.

38. A method of antigen presentation, the method comprising:
inducing, enhancing and/or maintaining prolonged survival of a cell in vitro;
incubating the cell with at least one antigen compound on a continuous basis; and
introducing at least one genetic modification within the cell to induce, enhance,
maintain and/or modify antigen-presentation by the cell.

39. The method of arrangement 38, wherein the inducing, enhancing and/or maintaining prolonged survival of the cell comprises co-culturing the cell with CD40L-expressing feeder cells.

40. The method of arrangement 39, further comprising co-culturing the cell with IL-2, IL-4 and IL-21.

41. The method of arrangement 38, wherein the inducing, enhancing and/or maintaining prolonged survival of the cell comprises expressing BCL-6 and BCL2-like 1 genes in the cell.

42. The method of arrangement 38, wherein the inducing, enhancing and/or maintaining prolonged survival of the cell comprises infecting the cell with EBV.

43. The method of arrangement 41 or 42, further comprising expressing CD40L gene in the cell.

44. The method of arrangement 43, wherein the BCL-6, BCL2-like 1, or CD40L gene is introduced into the cell through viral transduction, electroporation, squeezing, transfection, site-specific integration, transposons, CRISPR/Cas9, or TALEN.

45. The method of arrangement 38, wherein prolonged survival of the cell is at least three months in cell culture in vitro.

46. The method of arrangement 38, wherein the at least one antigen compound is encoded by one transgene encoding between one and forty polypeptides.

47. The method of arrangement 46, wherein each polypeptide comprises at least eight amino acids.

48. The method of arrangement 38, wherein the at least one antigen compound is stably expressed in the cell.

49. The method of arrangement 38, wherein the at least one antigen compound is transiently expressed in the cell.

50. A culturing mix comprising engineered cells according to any one of arrangements 22-37 and a culturing medium.

51. The method of arrangement 20, wherein the antigen compound is expressed from a polynucleotide that is integrated into the cell genome to achieve continuous expression.

52. The method of arrangement 20, wherein expression is at an effective level.

53. The method of arrangement 20, wherein the antigen is introduced once and that the cells can be expanded afterwards, and used multiple times to assess T cells without a need to deliver the antigen a second time.

54. The method of any one of the preceding arrangements involving BCL2-like 1, wherein BCL2-like 1 is BCL-xL.

55. A method of engineering a cell for antigen presentation, the method comprising:
inducing, enhancing and/or maintaining prolonged survival of a cell in vitro;
incubating the cell with at least one antigen compound for a sufficient duration to present antigen on a surface of the cell; and
introducing at least one genetic modification within the cell to induce, enhance, maintain and/or modify antigen-presentation by the cell.

56. The method of arrangement 55, wherein the cell comprises a primary human B cell.

57. The method of arrangement 56, wherein the primary human B cell is derived from peripheral blood.

58. The method of arrangement 56, wherein the primary human B cell is derived from a human tissue that contain B cells.

59. The method of arrangement 58, wherein the human tissues comprise tumor tissue, lymph nodes, spleen, cord blood, body fluids, and bone marrow.

60. The method of arrangement 56, wherein the primary human B cell is obtained by differentiation of a precursor cell.

61. The method of arrangement 60, wherein the precursor cell comprises hematopoietic stem cells.

62. The method of arrangement 56, wherein the primary human B cell is obtained by differentiation of induced pluripotent stem cells (iPSC).

63. The method of any of arrangements 55 to 62, wherein the inducing, enhancing and/or maintaining prolonged survival of the cell comprises co-culturing the cell with CD40L-expressing feeder cells.

64. The method of arrangement 63, further comprising co-culturing the cell with IL-2, IL-4 and IL-21.

65. The method of any of arrangements 55 to 62 or 64 or 10, wherein the inducing, enhancing and/or maintaining prolonged survival of the cell comprises expressing BCL-6 and BCL2-like 1 genes in the cell.

66. The method of any of arrangements 55 to 62, wherein the inducing, enhancing and/or maintaining prolonged survival of the cell comprises infecting the cell with EBV.

67. The method of arrangement 64, 65, or 66, further comprising expressing CD40L gene in the cell.

68. The method of any of arrangements 55 to 62, wherein the inducing, enhancing and/or maintaining prolonged survival of the cell comprises expressing BCL-6, BCL2-like 1, and CD40L genes in the cell.

69. The method of arrangement 68, wherein the BCL-6, BCL2-like 1, and CD40L genes can be on the same expression construct, or different expression construct.

70. The method of arrangement 65, 67, or 68, wherein the genes are introduced into the cell through viral transduction, electroporation, squeezing, transfection, site-specific integration, transposons, CRISPR/Cas9, or TALEN.

71. The method of any of arrangements 55 to 70, wherein the prolonged survival of the cell is at least three months in cell culture in vitro.

72. The method of any of arrangements 55 to 71, wherein the at least one antigen compound is encoded by one transgene encoding between one and forty polypeptides.

73. The method of arrangement 72, wherein each polypeptide comprises at least eight or nine amino acids.

74. The method of any of arrangements 55 to 73, wherein the at least one antigen compound is stably expressed in the cell.

75. The method of any of arrangements 55 to 73, wherein the at least one antigen compound is transiently expressed in the cell.

76. The method of any of arrangements 55 to 71, wherein the at least one antigen compound is electroporated into the cell.

77. The method of arrangement 76, wherein each polypeptide comprises at least eight or nine amino acids.

78. The method of arrangement 77, wherein the antigen compound is a polypeptide between 8 to 25 amino acids long.

79. The method of arrangement 78, wherein the polypeptide is 25 amino acids long.

80. The method of any of arrangements 55-79, wherein the antigen is presented to a T cell.

81. An engineered cell, wherein the cell:
has been adjusted to induce, enhance and/or maintain its survival in vitro;
has been electroporated with at least one antigen compound or has been engineered to express at least one antigen compound; and
has at least one genetic modification to induce, enhance, maintain or modify antigen-presentation by the cell.

82. The engineered cell of arrangement 81, wherein adjusted to induce, enhance and/or maintain its survival in vitro comprises expressing BCL-6 and BCL2-like 1 genes in the cell.

83. The engineered cell of arrangement 81, wherein adjusted to induce, enhance and/or maintain its survival in vitro comprises infecting the cell with EBV.

84. The engineered cell of arrangement 82 or 83, further comprising expressing CD40L gene in the cell.

85. The engineered cell of arrangement 82 or 84, wherein the genes are introduced into the cell through viral transduction, electroporation, squeezing, transfection, site-specific integration, transposons, CRISPR/Cas9, or TALEN.

86. The engineered cell of any of arrangements 81 to 85, wherein the cell survival is at least three months in cell culture in vitro.

87. The engineered cell of any of arrangements 81 to 86, wherein has been engineered to express at least one antigen compound comprises at least one transgene encoding between one and forty polypeptides wherein the polypeptides correspond to the at least one antigen compound.

88. The engineered cell of arrangement 87, wherein each polypeptide comprises at least eight amino acids.

89. The engineered cell of arrangement 87, wherein the at least one antigen compound is stably expressed in the cell.

90. The engineered cell of arrangement 81, wherein the at least one antigen compound is transiently expressed in the cell.

91. The engineered cell of any of arrangements 81 to 90, wherein the at least one antigen compound that has been electroporated into the cell is a polypeptide comprising at least eight or nine amino acids.

92. The engineered cell of arrangement 91, wherein the polypeptide is between 8 to 25 amino acids long.

93. The engineered cell of arrangement 93, wherein the polypeptide is 25 amino acids long.

94. The engineered cell of any of arrangements 32 to 36 or 81 to 93, wherein the engineered cell comprises a primary human B cell.

95. The engineered cell of arrangement 94, wherein the primary human B cell is derived from peripheral blood.

96. The engineered cell of arrangement 94, wherein the primary human B cell is derived from a human tissue that contain B cells

97. The engineered cell of arrangement 96, wherein the human tissues comprise tumor tissue, lymph nodes, spleen, cord blood, body fluids, and bone marrow.

98. The engineered cell of arrangement 94, wherein the primary human B cell is obtained by differentiation of a precursor cell.

99. The engineered cell of arrangement 98, wherein the precursor cell comprises hematopoietic stem cells.

100. The engineered cell of arrangement 94, wherein the primary human B cell is obtained by differentiation of induced pluripotent stem cells (iPSC).

101. A culturing mix comprising engineered cells according to any one of arrangements 81-100 and a culturing medium.

102. The method of arrangement 74, wherein the antigen compound is expressed from a polynucleotide that is integrated into the cell genome to achieve continuous expression.

103. The method of arrangement 74, 75, or 102, wherein expression is at an effective level.

104. The method of arrangement 74, 102, or 103, wherein the antigen is introduced once and that the cells can be expanded afterwards, and used multiple times to assess T cells without a need to deliver the antigen a second time.

105. The method of any one of the arrangements 55-80 or 102-104 involving BCL2-like 1, wherein BCL2-like 1 is BCL-xL.

106. The engineered cell of any one of the arrangements 22-36 or 81-100 involving BCL2-like 1, wherein BCL2-like 1 is BCL-xL.

107. The method or engineered cell of any of the above arrangements, wherein the antigen compound is a TCR antigen compound.

### EXAMPLES

### Example 1: Demonstration that Primary Human B Cells can be Efficiently Immortalized by BCL-6/BCL-XL or Epstein-Barr Virus (EBV) and Rapidly Expanded to Large Cell Numbers

Example 1 demonstrates that different immortalization methods can be used to immortalize primary human B cells isolated from peripheral blood mononuclear cells (PBMC) material and subsequently expand such immortalized B cells to high cell numbers in a short time-frames. Use of primary human B cells as antigen-presenting cells in TCR discovery and characterization studies can have several advantages: first, as described in Example 1, primary human B cells can be efficiently expanded to large cell numbers. Second, B cells have the capability to process antigen through both the MHC-class I and II antigen presentation pathway and disclosures herein describe further methods to achieve and improve such antigen-presentation. Third, if used in an autologous setting, B cells allow one to efficiently discover T cells and TCRs against all human leukocyte alleles (HLA) of a subject.

This example describes the efficient immortalization and expansion of primary human B cells by stable transduction with BCL-6/BCL-XL or infection of primary human B cells with Epstein-Barr Virus. Further experimental details are also provided in Example 10.

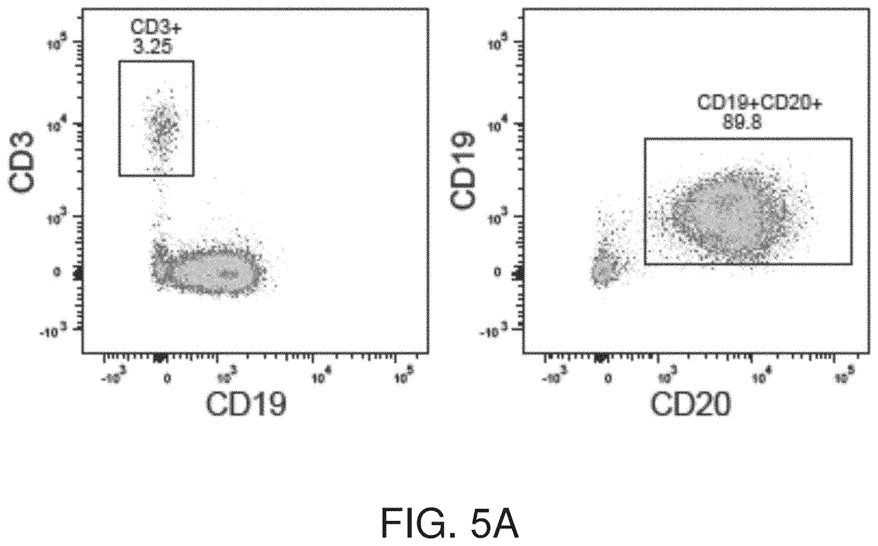

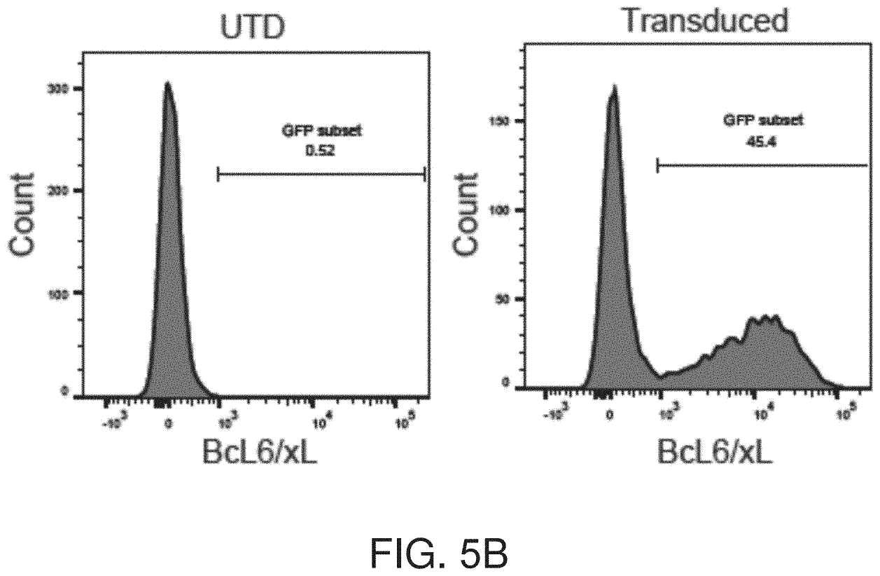

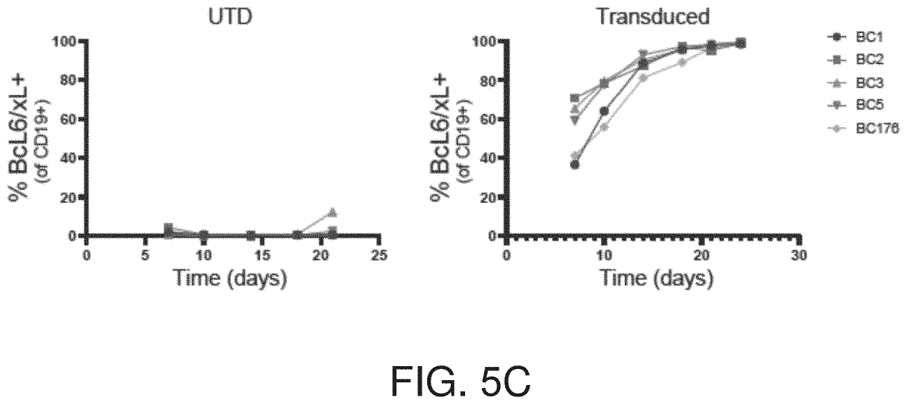

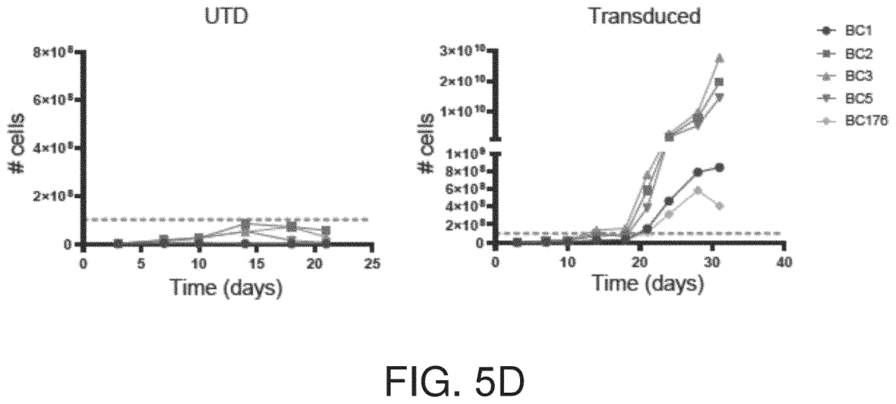

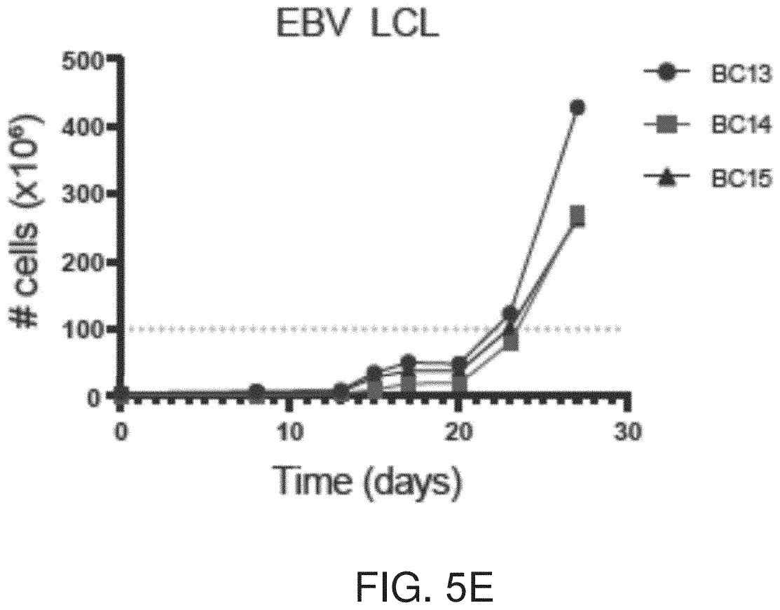

B cells are isolated from cryopreserved healthy donor PBMC using one of the available magnetic bead cell separation techniques known to the skilled artisan. In this example, the B cells are isolated using the MojoSort Human Pan B cell Isolation Kit (Biolegend) according to manufacturer's protocol. Briefly, PMBCs are incubated with biotin-labelled antibodies against CD2, CD3, CD14, CD16, CD16, CD36, CD56, CD123 and CD235. After incubation, magnetic streptavidin labelled nanobeads are added. After the second incubation, the tube containing the cells is put inside a magnet and the magnetically labelled fraction is retained on the sides of the tube and the liquid will contain the untouched B cells and can be aspirated from the tube for further use. (FIG. 5A). An ordinary person skilled in the art will appreciate that primary human B cells may be isolated by using either different isolation methods, such as flow cytometry sorting, as well as other phenotypic markers, including markers directly expressed by B cells for positive selection provided that such positive selection does not interfere with downstream processing of isolated B cells, including but not limited to interference with EBV infection or introduction of immortalization factors such as BCL-6/BCL-xL.

BCL-6/BCL-xL genes are expressed in germinal center B cells and allow them to proliferate without differentiating. Kwakkenbos et al. described a method to immortalize B cells by introducing these two genes into primary B cells and culture these cells in the presence of IL-21 and CD40L to stimulate the cells and expand them for a long period of time (Kwakkenbos et al. Nat Med 2010). The isolated B cells are activated by seeding human CD40L (hCD40L)-expressing L-cells in a TC-treated plate at least 4 hours before addition of B cells. CD40L expressing L cells are generated by retrovirally transducing murine L cells (ATCC) with the CD40L transgene. Retrovirus is generated by transfection of Phoenix-Eco producer cells and this virus is used to transduce L cells. CD40L transduced cells are subsequently selected by FACS-based sorting and irradiated with 50 Gy. Generally, between 20,000 and 200,000 isolated B cells are cultured on irradiated hCD40L-expressing L-cells at a ratio between 1:1-1:5. B cells are cultured in Complete B cell medium (RPMI1640 medium supplemented with 10% (v/v) Fetal Calf Serum (FCS), 1% (v/v) Penicillin/Streptomycin (P/S), 1% (v/v) Sodium Pyruvate, 1% (v/v) Non-essential amino acids, 1% (v/v) Glutamax, 50 nM 2-Mercaptoethanol) in the presence of rh-IL-2 (100 IU/mL) at a density of 0.1-0.25e6 B cells/mL.

After 48 hours, the culture medium is replaced with fresh complete B cell medium supplemented with rh-IL-2 (100 IU/mL), rh-IL-4 (50 ng/mL) and rh-IL-21 (50 ng/mL). After 24 hours, the activated B cells are immortalized by stable transduction with BCL-6/BCL-xL; protocols for retroviral transduction of primary B cells have been described in the literature (e.g. Kwakkenbos et al. Nat Med 2010) as well as in Example 10 are known to the skilled artisan. Retrovirally transduced B cells are cultured on irradiated human CD40L-transduced feeder cells in the presence of IL-21 (50 ng/mL) at cell densities between 200,000 and 250,000 cells/mL. CD40 receptor stimulation on B cells is essential to maintain primary human B cells in vitro. The BCL-6/BCL-xL construct contains a GFP marker which is used to monitor expression of BCL-6/BCL-xL on the immortalized, autologous B cells over time (FIG. 5B; FIG. 5C; FIG. 5D).

Another known immortalization method (as an alternative) is based on infection of the B cells by EBV which has been described in the literature, for example by Traggiai et al. Methods Mol Biol 2012. The B cells are isolated as described above and are cultured in complete B cell medium (RPMI1640 medium supplemented with 10% (v/v) Fetal Calf Serum (FCS), 1% (v/v) Penicillin/Streptomycin (P/S), 1% (v/v) Sodium Pyruvate, 1% (v/v) Non-essential amino acids, 1% (v/v) Glutamax, 50 nM 2-Mercaptoethanol) supplemented with 2×CpG ODN 2006 (final concentration of 2.5 ug/mL) to activate the Toll Like Receptors for subsequent EBV infection with 1 mL EBV-virus supernatant (ATCC) and the cells are incubated for 4 hours at 37° C. Cells are washed and resuspended in complete B cell medium supplemented with 2×CpG ODN 2006 (final concentration of 2.5 ug/mL) and 1% EBV virus supernatant. Approximately on day 3 and day 8, when the yellow medium indicates a low pH due to cell metabolism, cells are washed and resuspended in complete B cell medium without CpG. Once proliferation is visible by the presence of clumps of B cells, the cells are washed and cultured in RPMI1640 medium supplemented with 20% (v/v) FCS and 1% (v/v) P/S. Every 2 or 3 days the cells are split in a ratio of 1:2 and they are expanded for 4 weeks (FIG. 5E). The data shows that primary human B cells can be efficiently immortalized by BCL-6/BCL-XL or EBV and subsequently expanded to large cell numbers and maintained in culture for prolonged periods of time.

The results provided in FIG. 5A-5E demonstrate that primary human B cells can be efficiently immortalized by BCL-6/BCL-XL or EBV, including a description on isolation, immortalization and expansion of the B cells and growth kinetics. The results can be summarized as: FIG. 5A shows the isolation of B cells from PBMC material. B cells were isolated from healthy donor PBMC using the magnetic bead-based cell separation method from Mojosort. Untouched B cells were isolated using the pan B cell negative kit according to manufacturer protocol. Purity of the isolated B cells was measured using antibodies directed against CD3, CD19 and CD20. FIG. 5B applies to immortalization of B cells by stable transduction with BCL-6 and BCL-XL. A BCL-6-BCL-xL that also encodes GFP construct was transfected into Phoenix cells for virus production, and the resulting viral supernatant was used for retroviral transduction of activated B cells from 5 different healthy donors. Expression of BCL-6-BCL-XL was measured using the GFP signal. FIG. 5C applies to the expansion of immortalized B cells. The cells from 5B) were expanded by culturing the cells on irradiated hCD40L expressing L-cells in the presence of IL-21 (50 ng/mL final concentration) and split twice a week at a concentration of 0.2e6/mL cells. Expression of Bcl-6-Bcl-xL was measured using the GFP signal on the flow cytometer. FIG. 5D applies to the expansion of immortalized B cells. The cells from 5B) were expanded by culturing the cells on irradiated hCD40L expressing L-cells in the presence of IL-21 (50 ng/mL final concentration) and split twice a week at a concentration of 0.2e6/mL cells. Total cumulative cell counts were calculated based on counts that were measured while splitting the cells. FIG. 5E applies to the immortalization and expansion of B cells by EBV infection. Untouched B cells were isolated from healthy donor PBMC using the MojoSort magnetic bead-based cell separation method from Biolegend. Isolated B cells were immortalized by EBV infection and the cells were split every 2 or 3 days for a period of 4 weeks. Total cumulative cell counts were calculated based on counts that were measured while splitting the cells.

### Example 2: Demonstration that STAT5 Introduction into Primary Human B Cells Leads to Low B Cell Expansion

Example 2 demonstrates that immortalization of B cells with signal transducer and activator of transcription 5 (STAT5) leads to lower B cell expansion compared to BCL-6/BCL-xL immortalized B cells. This finding is relevant because while STAT5 has been described to inhibit differentiation of B cells and increase their life span (e.g. Scheeren et al. Plos One 2011) in a similar fashion to BCL-6/BCL-xL transduction, the presented data shows that it is a less preferred solution compared to BCL-6/BCL-xL and EBV immortalization.

B cells are isolated from cryopreserved healthy donor PBMC using a MojoSort Human Pan B Cell Isolation Kit described herein (Example 10).

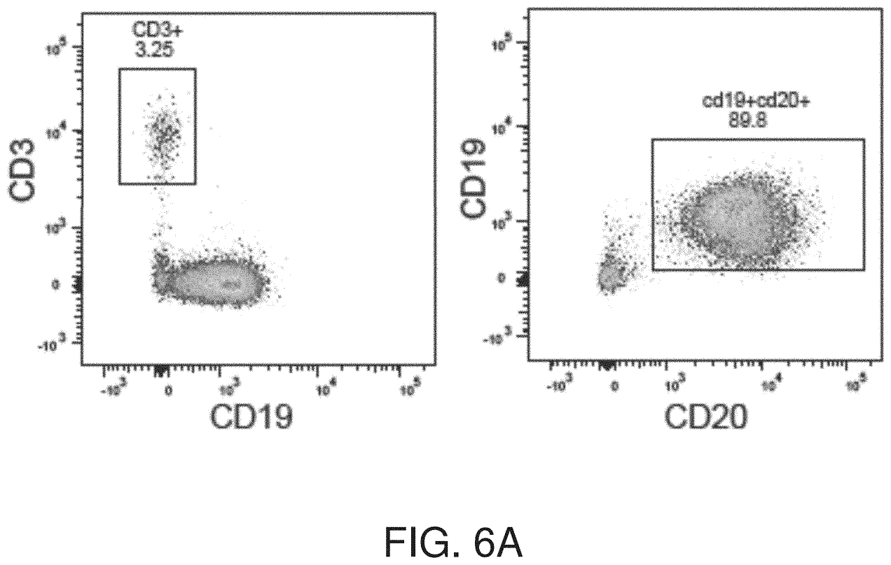

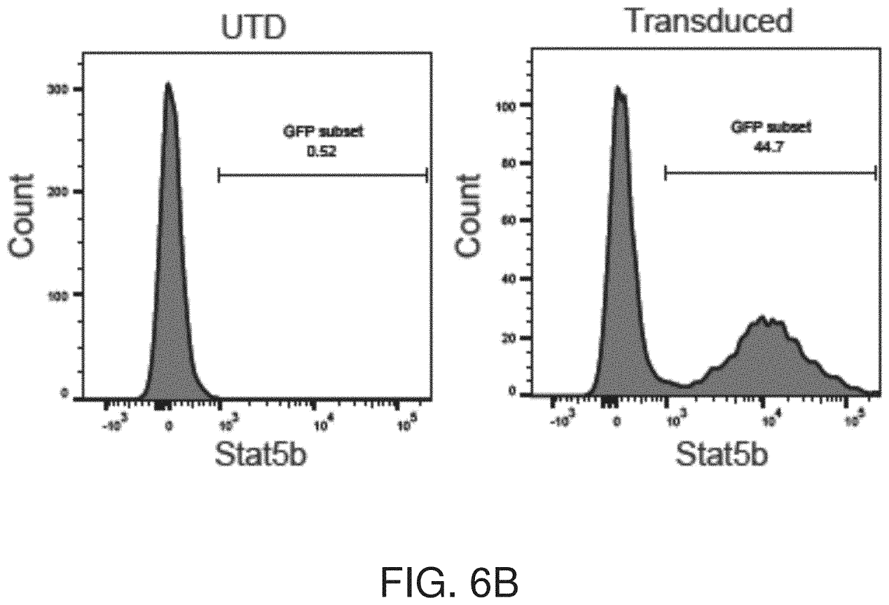

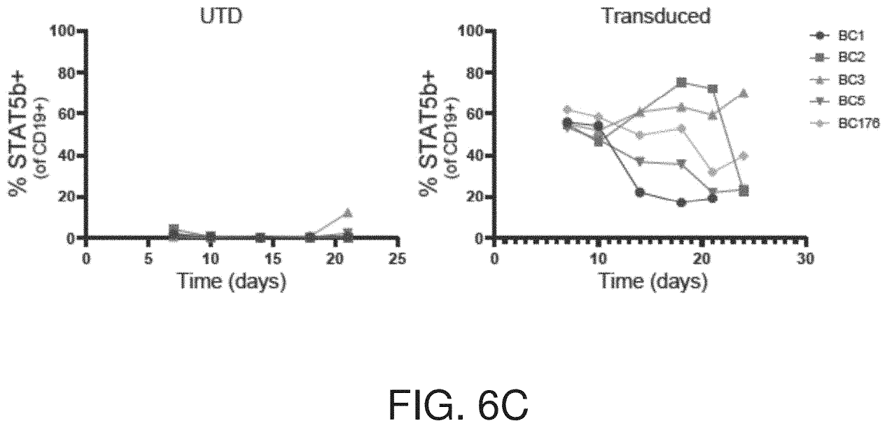

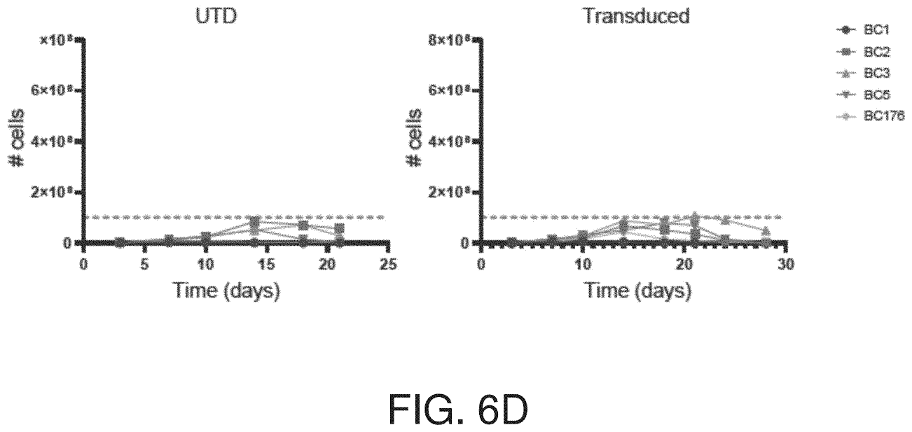

The isolated and activated B cells from the same healthy donor as described in Example 1 (FIG. 6A) are transduced with a murine-derived constitutively active STAT5 transgene, protocols for retroviral transduction of primary B cells have been described in the literature (e.g. Kwakkenbos et al. Nat Med 2010) and in this invention (Example 10). Retrovirally transduced B cells are cultured on irradiated human CD40L-transduced feeder cells in the presence of IL-21 (50 ng/mL). CD40 receptor stimulation on B cells is essential to maintain primary human B cells in vitro (see also example 7). The STAT5 retroviral construct contains a GFP marker which is used to monitor expression of STAT5 on the immortalized, autologous B cells over time (FIG. 6B-D). The data indicates that the total number of STAT5-expressing B cells does not meaningfully increase within 30 days of culture after immortalization compared to other B cell immortalization methods (FIG. 5).

As shown in FIG. 6A-6D, the present results demonstrate that STAT5 leads to less robust B cell expansion. As shown in FIG. 6A, which depicts the isolation of B cells from PBMC material, B cells were isolated from healthy donor PBMC using the magnetic bead-based cell separation method from Mojosort. Untouched B cells were isolated using the pan B cell negative kit according to manufacturer protocol. Purity of the isolated B cells was measured using antibodies directed against CD3, CD19 and CD20. As shown in FIG. 6B, which depicts results regarding immortalization of B cells by stable transduction with STAT5, the STAT5 construct was transfected into Phoenix cells for virus production, and the resulting viral supernatant was used for retroviral transduction of activated B cells from 5 different healthy donors. Expression of STAT5 was measured by GFP expression. As shown in FIG. 6C, which depicts the expansion and STAT5 expression on immortalized B cells, the cells from 6B) were expanded by culturing the cells on irradiated hCD40L expressing L-cells in the presence of IL-21 (50 ng/mL final concentration) and split twice a week at a concentration of 0.2e6/mL cells. Expression of STAT5 was measured by GFP expression. As shown in FIG. 6D, which depicts the expansion of immortalized B cells, the cells from 6B) were expanded by culturing the cells on irradiated hCD40L expressing L-cells in the presence of IL-21 (50 ng/mL final concentration) and split twice a week at a concentration of 0.2e6/mL cells. Total cumulative cell counts were calculated based on counts that were measured while splitting the cells.

### Example 3: Demonstration that CD40L Transgene Expression by B Cells can be Used as a Replacement for CD40L-Expressing Feeder Cells for Expansion

Example 3 demonstrates an improvement on the BCL-6/BCL-XL immortalization method by introduction of a CD40L transgene into BCL-6/BCL-xL transduced B cells. The data shows that CD40 receptor stimulation of B cells—a stimulation to maintain primary human B cells in vitro—can be achieved by direct transduction of B cells with a CD40L molecule rather than by co-culture of B cells with CD40L-transduced feeder cells. This modification significantly improves the industrial application by easing the maintenance of the immortalized B cells in cell culture and furthermore improves antigen-presentation by BCL-6/BCL-xL transduced B cells (Example 7 and 9).

CD40 receptor stimulation on B cells maintains primary human B cells in vitro. Typically, CD40L stimulation of BCL-6/BCL-XL immortalized B cells is typically performed by CD40L expressing feeder cells (L-cells) generated for example as described in Example 10. To ease the maintenance of the immortalized B cells in cell culture, we evaluated modification of B cells to express CD40L to allow for paracrine and/or autocrine CD40 receptor stimulation. Importantly, this eliminates the need to culture the B cells on CD40L expressing L-cells.

B cells were isolated from cryopreserved healthy donor PBMC using Miltenyi anti-CD20 microbeads and the autoMACS device (Miltenyi) as described in this invention (Example 10).

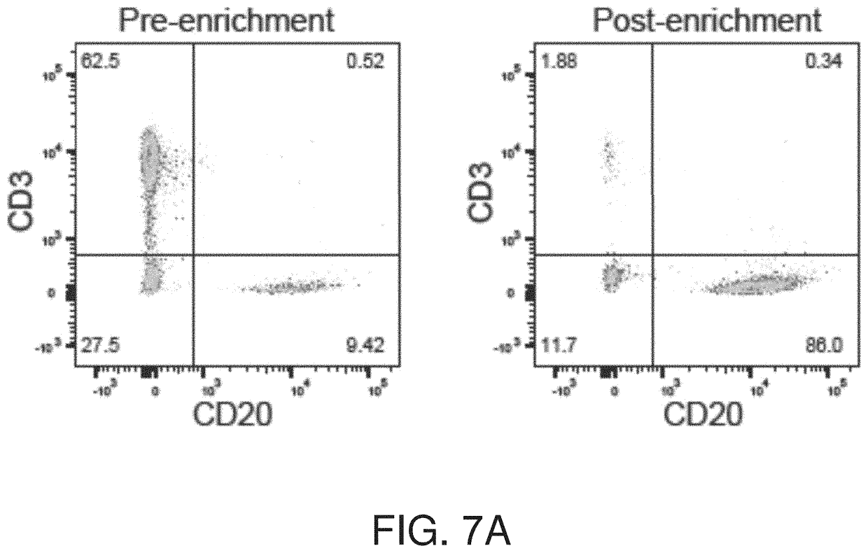

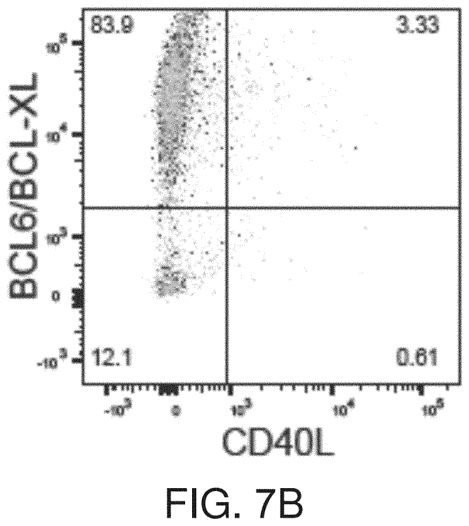

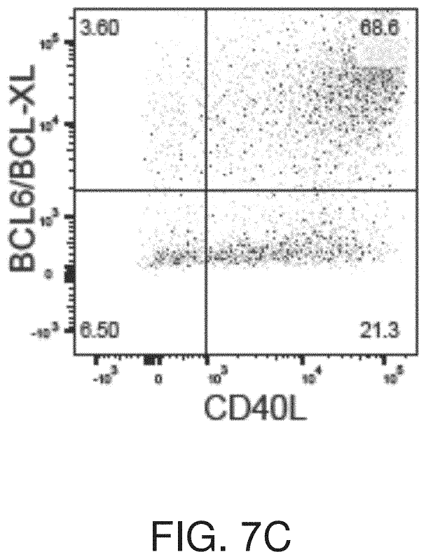

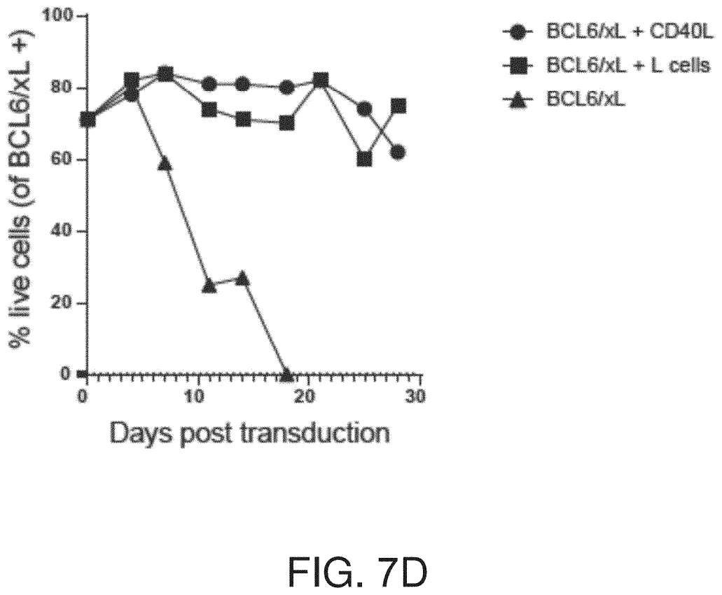

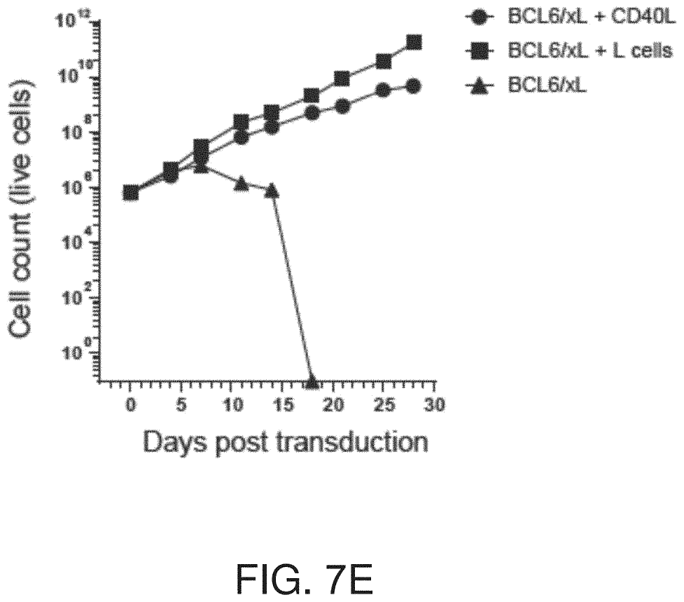

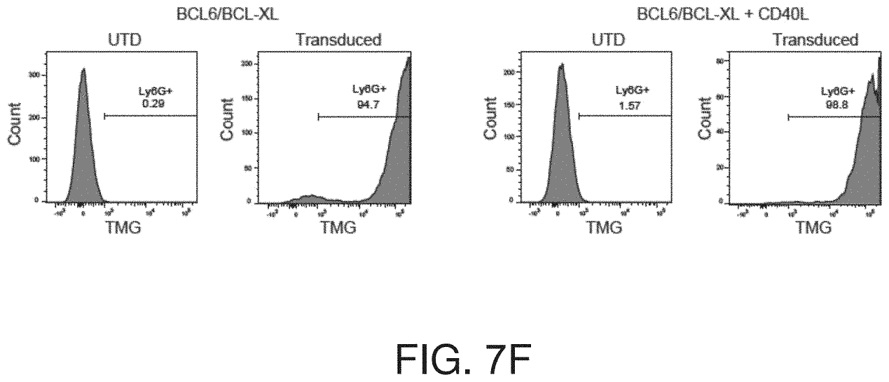

The activated, isolated B cells (FIG. 7A) were stably transduced with BCL-6/BCL-xL or a combination of BCL-6/BCL-XL and CD40L based protocols for retroviral transduction of primary B cells have been described in the literature (e.g. Kwakkenbos et al. Nat Med 2010) and as described in this invention (Example 10).

After 72 hours BCL-6/BCL-XL modified B cells were harvested, split and either cultured in the absence of L-cells or cultured on hCD40L expressing L cells. BCL-6/BCL-xL and CD40L transduced B cells are cultured in complete B cell medium supplemented with 50 ng/mL IL-21 in the absence of L-cells. The BCL-6/BCL-xL construct contains a GFP marker which is used to measure expression of BCL-6/BCL-xL on the immortalized B cells and monitor their proliferation over time (FIG. 7B; FIG. 7D; FIG. 7E). CD40L expression on the B cells is measured on a flow cytometer using an antibody directed against CD154 (FIG. 7C).

Delivery of the antigen in a Tandem-minigene (TMG) format by stable transduction allows one to utilize immortalized B cells as antigen-presenting cells (APCs). In order to demonstrate the possibility of introducing a TMG transgene into immortalized B cells, either BCL-6/BCL-xL or BCL-6/BCL-XL and CD40L transduced B cells were retrovirally transduced with a TMG construct using methods described in this invention (Example 10). In the retroviral construct the TMG is flanked by a LAMP-1 signaling domain and the transmembrane and truncated cytosolic domain and is linked to the cell surface expressed marker Ly6G and to a puromycin resistance gene using 2A-elements. Thus, it is possible to select for TMG-expression cells by culturing the cells in complete B cell medium supplemented with rh-IL21 (50 ng/mL) and 1 ug/mL puromycin for 3-4 days after which the expression of the TMG transgene was measured by using a Ly6G specific antibody (FIG. 7F). The data shows that CD40L transgene expression by B cells can be used as a replacement for CD40L-expressing feeder cells for expansion and that TMG transgenes can efficiently be introduced into both BCL-6/BCL-xL and BCL-6/BCL-XL/CD40L transduced B cells.

FIGS. 7A-7F demonstrate that CD40L transgene expression by B cells can be used as a replacement for CD40L-expressing feeder cells for expansion. FIG. 7A shows the isolation of B cells from PBMC material. B cells were isolated from healthy donor PBMC using the magnetic bead-based cell separation method from Miltenyi. CD20+ B cells were isolated using anti-CD20 microbeads and subsequent cell separation using the autoMACS according to manufacturer protocol. Purity of the isolated B cells was measured by FACS using antibodies directed against CD3 and CD20. FIG. 7B shows immortalization of B cells by stable transduction with BCL-6 and BCL-XL. A BCL-6-BCL-xL-GFP construct was transfected into 293Vec-Baev cells for virus production, and the resulting viral supernatant was used for retroviral transduction of activated B cells from a healthy donor. Expression of BCL6/BCL-xL was measured by FACS by GFP expression. FIG. 7C shows immortalization of B cells by stable co-transduction with BCL6 and BCL-XL and CD40L. The BCL6/BCL-xL construct and the CD40L construct were transfected into 293Vec-Baev cells for virus production, and the resulting viral supernatant was used for retroviral co-transduction of activated B cells from a healthy donor. Expression of BCL6/BCL-xL was measured by GFP expression and expression of CD40L was measured using an antibody directed against CD154, analyzed by FACS. FIG. 7D shows expansion of immortalized B cells in presence or absence of CD40L. The cells from 7B and 7C) were expanded by culturing the cells for 3 days on irradiated hCD40L expressing L-cells in the presence of IL-21 (50 ng/mL final concentration). After 72 hours, B cells were harvested, and half of the BCL-6/BCL-xL cells were cultured on hCD40L expressing L cells while the other half, as well as the BCL-6/BCL-xL/hCD40-L cells were taken of L cells. Cells were split twice a week at a concentration of 0.2e6/mL cells and cell viability was analyzed. FIG. 7E shows expansion of immortalized B cells in presence or absence of CD40L. The cells from 7B and 7C) were expanded by culturing the cells for 3 days on irradiated hCD40L expressing L-cells in the presence of IL-21 (50 ng/mL final concentration). After 72 hours, B cells were harvested, and some of the BCL-6/BCL-xL cells were cultured on CD40L expressing L cells, while other BCL-6/BCL-xL cells, as well as BCL-6/BCL-xL/hCD40L cells, were not. Cells were split twice a week at a concentration of 0.2e6/mL cells. The total cumulative cell counts was calculated based on counts that were measured while splitting the cells. FIG. 7F shows stable transduction of a TMG on BCL-6/BCL-XL and BCL-6/BCL-XL-CD40L immortalized B cells after puromycin (1 ug/mL) selection, a method known to the skilled artisan and described in Example 10. The TMG construct was transfected into 293Vec-Baev cells for virus production, and the resulting viral supernatant was used for retroviral transduction of immortalized B cells from 7B). Expression of TMG was measured by FACS using an antibody directed against Ly6G. Gating strategy: Lymphocytes>live cells>CD20+ cells>GFP+ cells>Ly6G.

Amino acid sequence for a hCD40L transgene
(SEQ ID NO: 10):
MIETYNQTSPRSAATGLPISMKIFMYLLTVFLITQMIGSALFAVYLHRRL
DKIEDERNLHEDFVFMKTIQRCNTGERSLSLLNCEEIKSQFEGFVKDIML
NKEETKKENSFEMQKGDQNPQIAAHVISEASSKTTSVLQWAEKGYYTMSN
NLVTLENGKQLTVKRQGLYYIYAQVTFCSNREASSQAPFIASLCLKSPGR
FERILLRAANTHSSAKPCGQQSIHLGGVFELQPGASVFVNVTDPSQVSHG
TGFTSFGLLKL*

### Example 4: Stable Transduction and Selection of Immortalized Human B Cells Expressing an Antigen-Encoding Transgene

Example 4 demonstrates that immortalized B cells can be stably transduced with transgenes encoding antigens and that surface or antibiotic selection markers included in the polycistronic vector enable efficient detection and enrichment of these antigen-expressing B cells. Stable expression of antigen is beneficial because it eases the production of larger numbers of antigen-presenting B cells. Alternative antigen delivery methods, such as exogenous peptide loading, electroporation with an antigenic compound (e.g. mRNA, DNA, peptide, protein) would require substantially more handling work compared to stably modification of small numbers of B cells and their subsequent expansion. Furthermore, stable antigen expression ensures high and continued expression of antigen and likely reduces heterogeneity in antigen-expression levels within the B cell population.

The transduction and selection of antigen-expressing immortalized B cells is needed to detect neo-antigen reactive TCRs as they are used as APCs in our TCR isolation platform.

The transgenes encoding the patient-specific mutations are made in a tandem minigene format (TMG). These constructs include up to 12 tumor-specific mutations as 25 mer concatenated polypeptide minigenes and also include LAMP-1 signaling and transmembrane domains.

This example shows the generation of antigen-expressing, immortalized B cells using different immortalization techniques and stable transduction with transgenes encoding patient-specific mutations in a tandem-minigene format (TMG). The TMG-encoding vector additionally contains both a Ly6G protein as well as a puromycin-resistance gene allowing to select TMG-modified B cells by antibiotic selection and detect TMG-expression by flow cytometry using a Ly6G-specific antibody.

EBV-immortalized B cells, derived from a patient with mismatch-repair proficient colorectal cancer (MMRp-CRC) are cultured in RPMI1640 medium supplemented with 20% (v/v) Fetal Calf Serum (FCS) and 1% (v.v) Penicillin/Streptomycin (P/S) at a density of 0.3-0.6e⁶ cells/mL.

In order to retrovirally transduce B cells with Tandem-minigene (TMG)-transgenes protocols known by the skilled artisan and described in this invention (Example 10) are used. TMGs include up to 12 tumor-specific mutations encoded as 25mer concatenated polypeptide minigenes and also include LAMP-1 signaling and transmembrane domains as well as a puromycin resistance gene and a Ly6G marker to enable efficient selection and detection of TMG-expressing B cells.

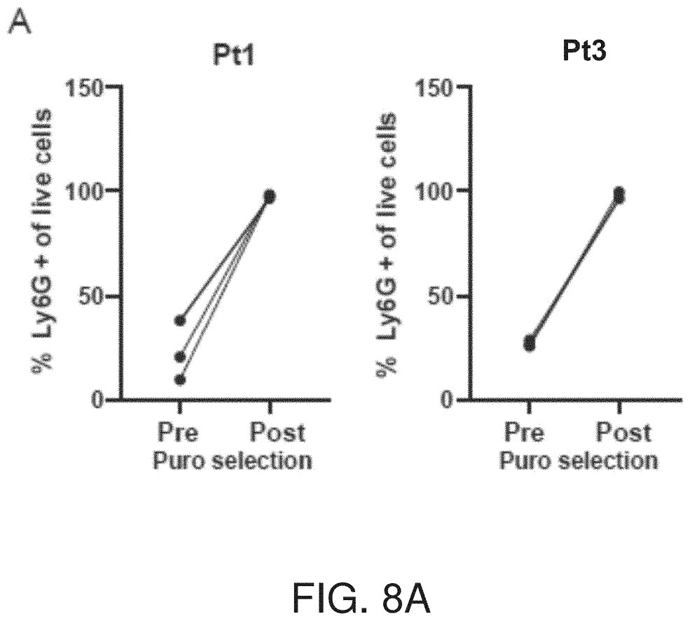

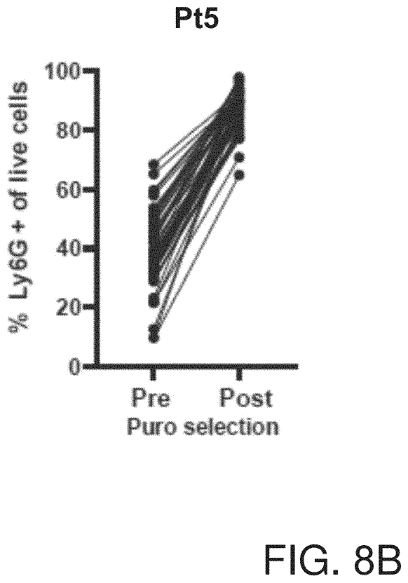

The frequency of TMG-expressing B cells is measured by flow cytometry using a fluorochrome labeled Ly6G-specific antibody on day 4 post-transduction. Subsequently, cells are cultured in media containing puromycin at a concentration of 5 ug/mL for 4 days. To determine the frequency of TMG-expressing B cells after puromycin-based selection is measured by flow cytometry using a fluorochrome labeled Ly6G-specific antibody (FIG. 8A)

In addition, B cells from a patient with basal cell carcinoma are isolated from PBMCs by negative selection and subsequently immortalized by BCL-6/BCL-XL and CD40L as described in this application (Example 3 and 10). Subsequent, transduction of the immortalized B cells with transgenes encoding TMG transgene is performed using methods known to the skilled artisan and as described in this invention (Example 10). The frequency of TMG-expressing B cells is measured on day 4 post-transduction by flowcytometry using a fluorescently-labelled, Ly6G specific antibody. Subsequently, cells are cultured in media containing puromycin (1 ug/mL) for 4 days. after which the purity is measured by flowcytometry. To determine the frequency of TMG-expressing B cells after puromycin-based selection is measured by flow cytometry using a fluorochrome labeled Ly6G-specific antibody (FIG. 8B). The data in this example shows that both EBV-immortalized B cells as well as BCL-6/BCL-XL immortalized B cells can be stably transduced with transgenes encoding tumor-specific mutations in a tandem-minigene format and that antibiotic selection can be used to obtain highly enriched populations of TMG-expressing B cells (FIGS. 8A and 8B).

With respect to further details of this experiment and the results provided in FIGS. 8A and 8B, it is noted that they demonstrate stable transduction and selection of immortalized human B cells. FIG. 8A demonstrates transduction and selection of EBV immortalized B cells with TMG-encoding constructs. EBV-immortalized B cells were virally transduced by spin-based transduction. 4 days post transduction, cells were analyzed for Ly6G expression by flowcytometry. Cells were then selected by adding the antibiotic Puromycin to the culture medium and re-analyzed for Ly6G expression FIG. 8B demonstrates transduction and selection of BCL-6/BCL-XL immortalized B cells with TMG-encoding constructs. Primary B cells were isolated from PBMCs and immortalized by introducing BCL-6/BCL-XL and CD40L. After immortalization, B cells were virally transduced with patient-specific TMG constructs, after 4 days transduction efficiencies were measured by analyzing Ly6G-expression by flowcytometry. Subsequently cells were supplemented with puromycin to allow selection of transduced cells and were re-analyzed by flowcytometry for Ly6G expression.

### Example 5: Demonstration that Inclusion of LAMP1 Signaling and Transmembrane Sequences and CD40L Expression by Immortalized B Cells Enhances Antigen-Presentation of MHC-Class II Restricted T Cell Epitopes

Example 5 demonstrates that inclusion of LAMP1 signaling and transmembrane sequences within TMG transgene sequences enhance presentation of MHC-class I and II restricted T cell epitopes by both EBV and/or BCL-6/BCLXL immortalized B cells.

This is a useful improvement over the current state of the art, in at least that typically different antigen formats are used to achieve optimal antigen presentation in the MHC-class I and II pathways, respectively. Optimizing antigen-presentation in both pathways is of use for TCR discovery efforts aiming to detect MHC-class I and II restricted T cell receptors. The example presents different TMG designs encoding varying numbers of 25mer epitopes (1, 3, 12 or 40 epitopes) with or without LAMP1 signaling and transmembrane sequences. The TMG-encoding transgene further includes a puromycin-resistance gene and GFP allowing rapid detection and enrichment of TMG-expressing B cells based on antibiotic resistance. In in this example, the impact of CD40L expression on MHC-class I and II antigen presentation by BCL-6/BCL-XL immortalized B cells is evaluated.

B cells from healthy donors are isolated from PBMCs using magnetic-bead enrichment using protocols known to the skilled artisan and subsequently immortalized either by EBV infection or transduction with BCL-6/BCL-xL with methods known to the skilled artisan and as described herein (Example 10). Non-EBV immortalized B cells are either transduced with BCL-6/BCL-xL alone or co-transduced with CD40L transgene. Retroviral supernatant for B cell transduction is produced using methods known to a person skilled in the art and as described in this invention (Example 10). B cells are retrovirally transduced with different TMG constructs. The example presents different TMG designs encoding varying numbers of 25mer epitopes (1, 3, 12 or 40 epitopes) with or without LAMP1 signaling and transmembrane sequences. 96 hours after transduction, TMG-expressing B cells are enriched based on puromycin resistance using protocols described in the art and this invention (Example 10). In brief, BCL-6/BCL-xL immortalized B cells are cultured in complete B cell medium (RPMI1640 medium supplemented with 10% (v/v) Fetal Calf Serum (FCS), 1% (v/v) Penicillin/Streptomycin (P/S), 1% (v/v) Sodium Pyruvate, 1% (v/v) Non-essential amino acids, 1% (v/v) Glutamax, 50 nM 2-Mercaptoethanol) containing rh-IL-21 (50 ng/mL) and 1 ug/ml puromycin and at a density of 200,000-250,000 cells/ml for 96 hours. In some cases, where no cell death is observed within 48 hours after addition of puromycin to the medium, 1 ug/ml puromycin is added to the culture. EBV-immortalized B cells are cultured in medium (RPMI1640 medium supplemented with 20% (v/v) Fetal Calf Serum (FCS), 1% (v/v) Penicillin/Streptomycin (P/S)) containing 5 ug/ml puromycin for 96 hours. In some cases, where no cell death is observed within 48 hours after addition of puromycin to the medium, 5 ug/ml puromycin is added to the culture.

In parallel, Jurkat T cells are transduced with various T cell receptor genes using protocols known to the skilled artisan. In brief, TCR-encoding plasmids are transfected into Phoenix-Ampho virus producer cells (ATCC) using Fugene transfection reagent and protocols known to the skilled artisan. The resulting retroviral virions are used to transduce Jurkat reporter T cells. The Jurkat reporter T cells are modified to express human CD8α and CD8β after transduction with a CD8α-P2A-CD8β transgene (SEQ ID NO: 2) using methods known to the skilled artisan.

SEQ ID NO: 2:
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYVGSGATNFSLLKQAGD
VEENPGPMRPRLWLLLAAQLTVLHGNSVLQQTPAYIKVQTNKMVMLSCEA
KISLSNMRIYWLRQRQAPSSDSHHEFLALWDSAKGTIHGEEVEQEKIAVF
RDASRFILNLTSVKPEDSGIYFCMIVGSPELTFGKGTQLSVVDFLPTTAQ
PTKKSTLKKRVCRLPRPETQKGPLCSPITLGLLVAGVLVLLVSLGVAIHL
CCRRRRARLRFMKQFYK

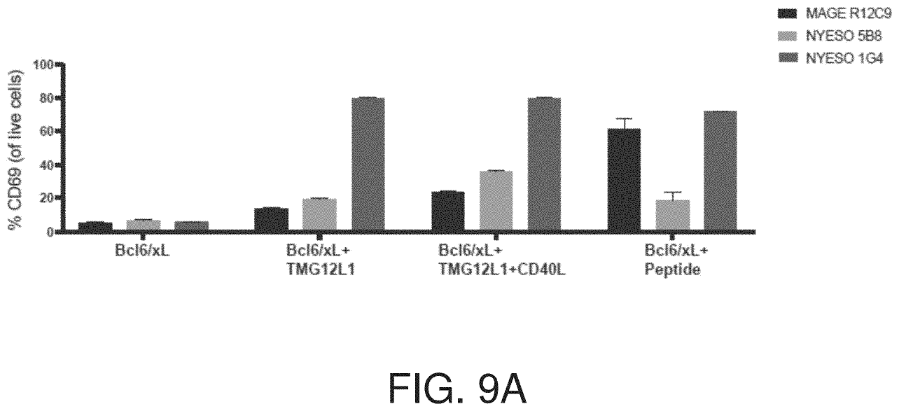

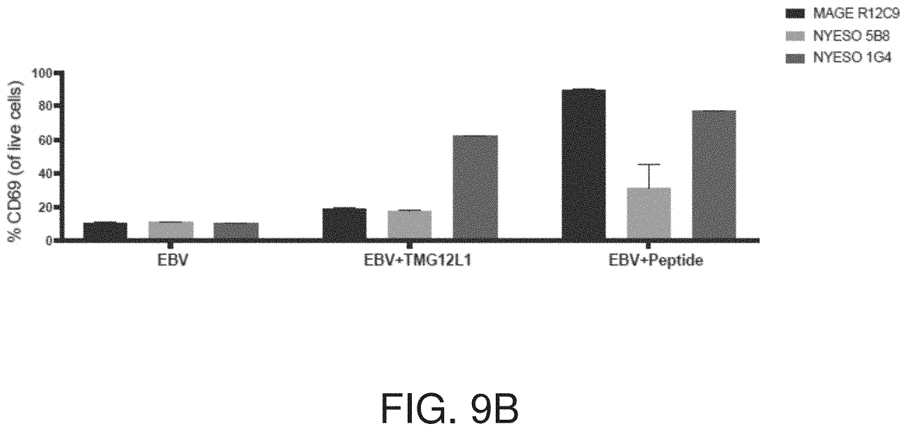

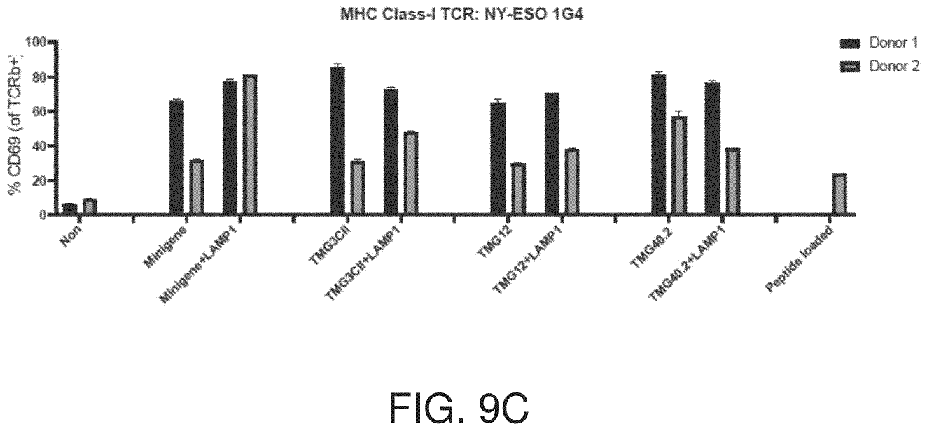

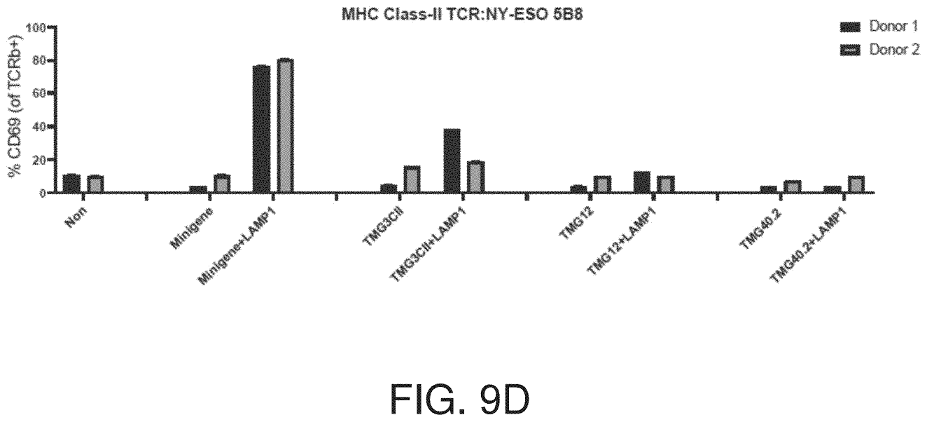

The use of murine TCR constant domain sequences as incorporated in the TCR transgene allows for the detection of TCR-modified Jurkat T cells by flow cytometry using a murine TCRβ constant domain specific antibody. The TCR-modified are sorted to enrich this population before performing a co-culture with these cells (FIG. 9A; FIG. 9B) or in some situations these cells are not sorted and the % of TCR+ varies between 25-80% (FIGS. 9C and 9D). In this example TCR sequences with specificity for the following T cell epitopes are used: MAGE-A3 (R12C9 epitope; MHC-class II restricted T cell epitope), NY-ESO-1 (5B8 epitope; MHC-class II restricted T cell epitope) and NY-ESO-1 (1G4 epitope; MHC-class I restricted T cell epitope) and GCN1L1 (GCN1L1 epitope; MHC-class I restricted T cell epitope).

48 hours prior to co-culture with immortalized B cells, TCR transduced Jurkat cells are seeded at a low density (1e5/mL) in culture medium (RPMI1640 medium supplemented with 10% (v/v) Fetal Calf Serum and 1% (v/v) Penicillin/Streptomycin (P/S)). In order to assess antigen-presentation by B cells, B cells and TCR transduced Jurkat cells are cultured in a 1:1 E:T ratio (200,000 cells in 200 ul culture media per well in a 96-well microtiter plate) for 20 hours. As a positive control, B cells are loaded with either 9mer peptides (MHC-class I epitopes) by incubation with 10 ug/mL peptide or 25mer peptides (MHC-class II epitopes) by incubation with 20 ug/mL peptide o/n at 37 C. EBV-immortalized B cells are peptide-loaded with 25-mer peptides at a cell density of 0.5e6/mL cells and BCL-6/BCL-xL immortalized B cells are peptide-loaded at a cell density of 0.75e6/mL. EBV and BCL-6/xL immortalized B cells are peptide-loaded with 10-mer peptides at a cell density of 1e6/mL cells. Peptide-loaded B cells are washed prior to co-culture with TCR transduced Jurkat T cells. After 20 hours, CD69 expression by TCR transduced Jurkat cells is measured by flowcytometry. The data demonstrates that expression of CD40L by immortalized B cells can enhance MHC-class II restricted antigen-presentation (FIGS. 9A and 9B) and that addition of LAMP1 signaling and transmembrane sequences improves MHC-class II restricted antigen-presentation by B cells stably expressing the antigen, e.g. as a TMG-transgene (FIGS. 9C and D). Furthermore, the data shows that MHC-class II restricted antigen presentation may be impaired if more than twelve 25mer polypeptides are encoded in a single TMG transgene (FIG. 9D).

Further details regarding the results in FIG. 9A-D are provided for the demonstration that antigen-expressing B cells can stimulate both MHC-class I and class II restricted TCR transduced T cells with addition of CD40L and LAMP1. In FIG. 9A, which assesses T cell reactivity towards BCL-6/BCL-XL immortalized B cells with or without CD40L transduced with TMG, immortalized B cells were co-transduced with CD40L and subsequently transduced with a TMG encoding 12 epitopes and LAMP1 sequences. TCR transduced Jurkats directed against the epitopes were then co-cultured with the B cells and reactivity as measured by the activation marker CD69 was assessed by flowcytometry. In FIG. 9B, which assesses T cell reactivity towards EBV immortalized B cells with or without transduced with TMG, EBV immortalized B cells were transduced with a TMG encoding 12 epitopes and LAMP1 sequences. TCR transduced Jurkats were then co-cultured with the B cells and reactivity as measured by the activation marker CD69 was assessed by flowcytometry. In FIG. 9C, which shows Class-I restricted T cell reactivity towards BCL-6/BCL-XL immortalized B cells expressing TMGs with or without LAMP1 signaling and transmembrane sequences, immortalized B cells from two different healthy donors were transduced with TMG constructs encoding a range of epitopes in the presence or absence of LAMP1. TCR transduced Jurkat T cells were co-cultured with the B cells overnight and subsequently analyzed for upregulation of CD69 by flowcytometry. In FIG. 9D, which shows class-II restricted T cell reactivity towards BCL-6/BCL-XL immortalized B cells expressing TMGs with or without LAMP1 signaling and transmembrane sequences, immortalized B cells from two different healthy donors were transduced with TMG constructs encoding a range of epitopes in the presence or absence of LAMP1. TCR transduced Jurkat T cells were co-cultured with the B cells overnight and subsequently analyzed for upregulation of CD69 by flowcytometry

### Example 6: Demonstration that Exogenous Loading of Immortalized B Cells with 25mer Polypeptides is Unsuited for TCR Discovery of MHC-Class I Restricted TCRs

The discovery of MHC-class II restricted T cells and TCR genes is of interest for various therapeutic applications, including cancer therapy. As provided herein, there can be B cells that stably express a T cell antigen and thereby efficiently present antigen through both MHC-class I and II presentation pathway. A conceivable alternative would be the exogenous loading of 25mer peptides onto B cells which can bind to MHC-class II molecules as well as MHC-class I molecules if processed by cellular proteases to a length suitable for binding to MHC-class I molecules (approx. 9-11mer length).

This example demonstrates that exogenous loading of B cells with 25mer polypeptides at titrating amounts—unlike peptide loading with shorter (9- or 10-mer) polypeptides encoding the relevant MHC-class I restricted epitope—does not achieve efficient antigen-presentation for MHC-class I restricted T cell epitopes. Therefore, exogenous loading of 25mer polypeptides is not a preferred solution when using human B cells as antigen-presenting cells for TCR discovery.

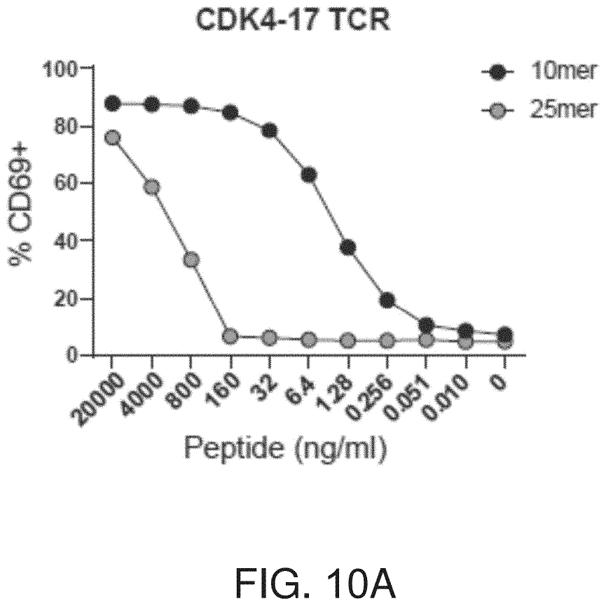

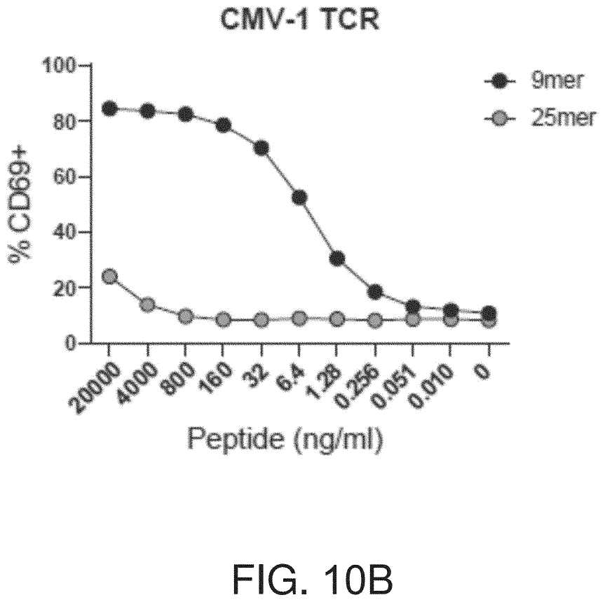

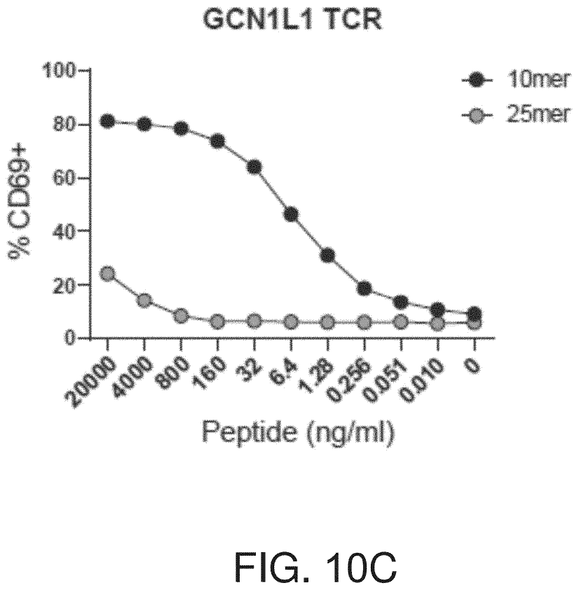

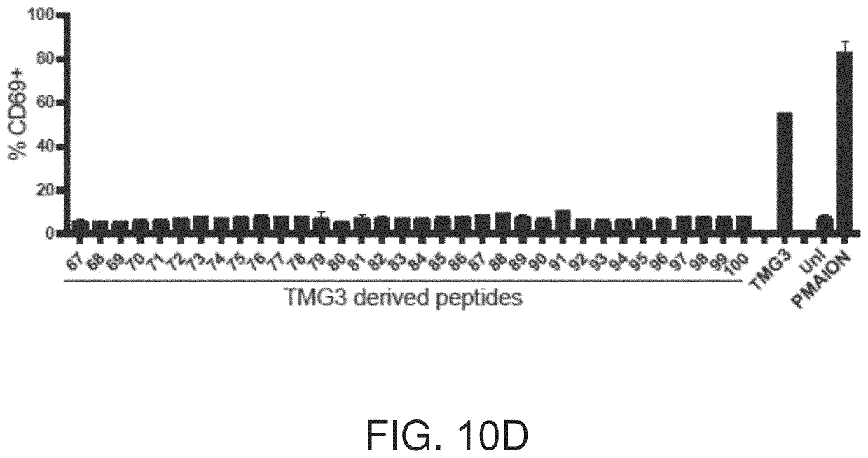

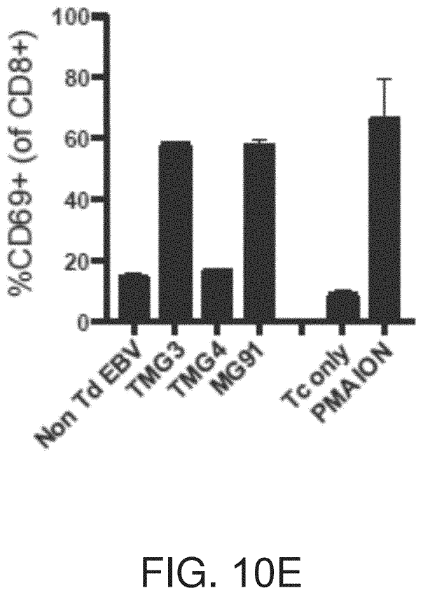

EBV immortalized B cells are loaded at a density of 0.5e6 cells/mL with either (i) titrating amounts of 25-mer peptides (we need to add sequences) overnight at 37 C or at a cell density of 1e6 cells/mL with (ii) 9- or 10-mer peptides derived from the 25mer peptides (add sequences) for 90 mins at 37 C. After exogenous peptide loading, APCs are washed extensively and co-cultured with TCR transduced Jurkat T cells. In order to generate TCR transduced Jurkat T cells, TCR-encoding plasmids are transfected into Phoenix-Ampho virus producer cells (ATCC) using Fugene transfection reagent and protocols known to the skilled artisan. The resulting retroviral virions are used to transduce a Jurkat reporter T cell line. The Jurkat reporter T cell line lacks endogenous TCR expression (generation of such a genetic knock-out being described for example in Mezzadra et al Nature 2017) and is modified to express human CD4, CD8α and CD8β after transduction with a CD8α-P2A-CD8β-T2A-CD4 transgene using methods known to the skilled artisan. The use of murine TCR constant domain sequences as incorporated in the TCR transgene allows for the detection of TCR-modified Jurkat T cells by flow cytometry using a murine TCRβ constant domain specific antibody. The TCR transduced Jurkat T cells (47-67% TCR+) are cultured at low density 48 hours prior to the assay. The APCs and TCR transduced Jurkat T cells are co-cultured at a 1:1 ratio (200.000 cells in 200 ul culture media per well in a 96-well microtiter plate) overnight at 37 C. Subsequently, CD69 expression by TCR transduced Jurkat T cells is analyzed. The data indicates that for three MHC-class I restricted TCR genes specific for CMV pp65, mutated CDK4 and mutated GCN1L1 exogenous loading with 25mer-polypeptides only leads to Jurkat T cell activation at higher concentrations (FIG. 10A-C).

In a further assay, 33 different 25mer polypeptides encoded in a single TMG-transgene (TMG3) are synthesized and exogenously loaded on EBV-immortalized B cells as described above. Peptide-loaded B cells are co-cultured with Jurkat T cells expressing a TCR with specificity for polypeptide 91 encoded in the TMG as described above. B cells expressing TMG3 and a transgene encoding polypeptide 91 (MG91) are used as additional controls and are generated as described in Example 10 by retroviral transduction and subsequent enrichment based on antibiotic resistance. Notably, while TCR transduced Jurkat T cells are activated by TMG3- and MG91-expressing B cells, B cells exogenously loaded with the relevant 25-mer polypeptide do not lead to CD69 upregulation by TCR transduced Jurkat T cells as measured by flow cytometry (FIGS. 10D and 10E). Thus, this data further suggests that exogenous loading of 25mer polypeptides is unsuited to reliably identify and characterize MHC-class I restricted T cells and TCRs.

Additional details regarding FIG. 10A-D, regarding their demonstration that exogenous loading of immortalized B cells with 25mer polypeptides is unsuited for TCR discovery of MHC-class I restricted TCRs, is provided below. As shown in FIG. 10A, CDK4-17 TCR transduced T cell reactivity towards exogenously loaded EBV immortalized B cells. Immortalized B cells were loaded with titrating amounts of peptide of different lengths encoding the cognate antigen and subsequently cultured with TCR transduced T cells. CD69 upregulation was assessed by flowcytometry. As shown in FIG. 10B, CMV TCR TCR transduced T cell reactivity towards exogenously loaded EBV immortalized B cells. Immortalized B cells were loaded with titrating amounts of peptide of different lengths encoding the cognate antigen and subsequently cultured with TCR transduced T cells. CD69 upregulation was assessed by flowcytometry. As shown in FIG. 10C, GCN1L1 TCR transduced T cell reactivity towards exogenously loaded EBV immortalized B cells. Immortalized B cells were loaded with titrating amounts of peptide of different lengths encoding the cognate antigen and subsequently cultured with TCR transduced T cells. CD69 upregulation was assessed by flowcytometry. FIG. 10D indicates TCR validation on peptides encoding single mutations present in TMG. EBV immortalized B cells were loaded with 25mer peptides encoding the single mutations present in the TMG construct. Subsequent co-incubation with pure population of the TCR transduced T cells show no reactivity towards any peptide. FIG. 10E indicates TCR validation of minigene constructs encoding single mutations present in TMG. EBV immortalized B cells were retrovirally transduced with constructs encoding the single mutations present in the TMG construct. Co-incubation with pure population of the TCR transduced T cells shows reactivity towards 1 epitope.

### Example 7: Demonstration that Expression of a CD40L Transgene can Enhance Both MHC-Class I and II Antigen-Presentation by Immortalized B Cells

Example 7 provides evidence that introduction of CD40L transgene into BCL-6/BCLXL immortalized B cells can enhance antigen-presentation through both the MHC-class I and II presentation pathway. This finding is relevant when B cells are used for TCR discovery and characterization studies.

This example describes the activation of TCR transduced T cells by antigen-expressing immortalized B cells.

B cells from two healthy donors are isolated and immortalized by transduction with BCL-6/BCL-xL as described in Example 5 and 10 of this invention. In this Example, B cells are either transduced with BCL-6/BCL-xL alone or in combination with a CD40L transgene. After immortalization B cells are transduced with different TMG constructs encoding different numbers of 25mer polypeptides (1, 3, 12 or 40) and including LAMP1 signaling and transmembrane sequences. 96 hours post-transductions TMG-expressing B cells are enriched by antibiotic resistance selection as described in Examples 5 and 10. B cells are cultured as previously described, at a density of 0.2-0.25e6 cells/mL in the presence of rh-IL-21 (50 ng/mL).

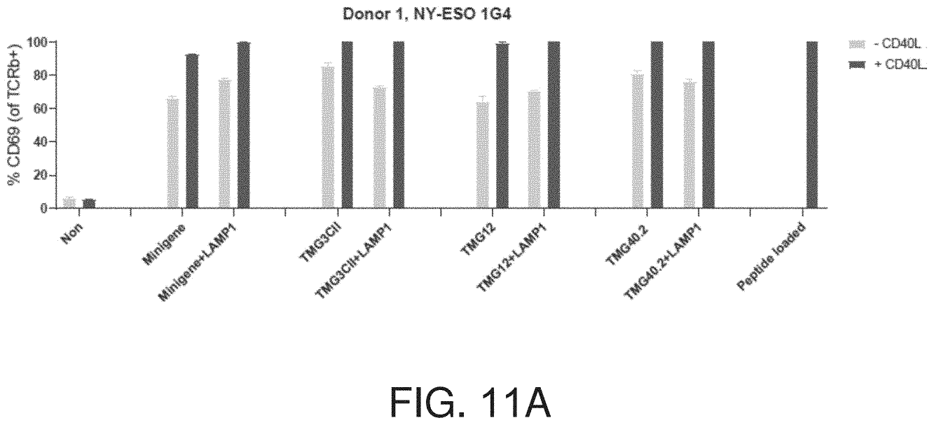

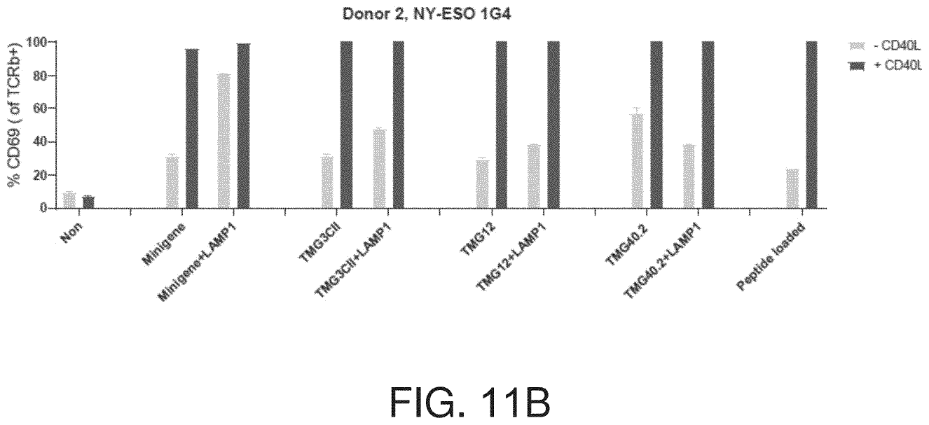

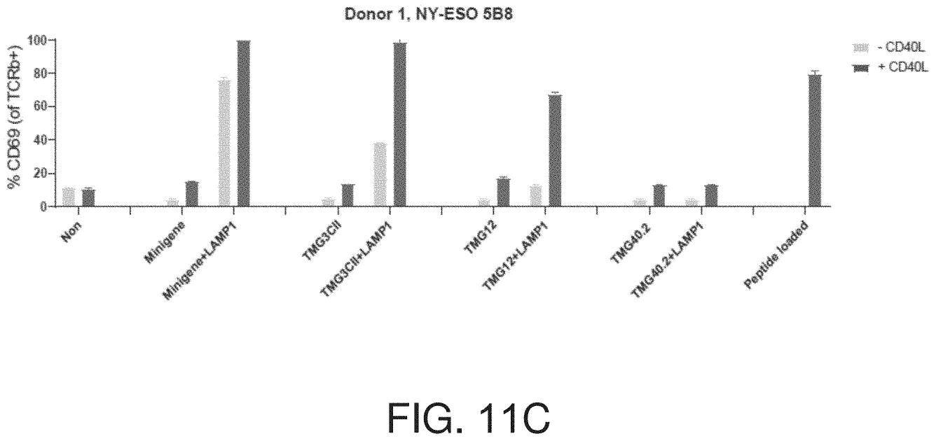

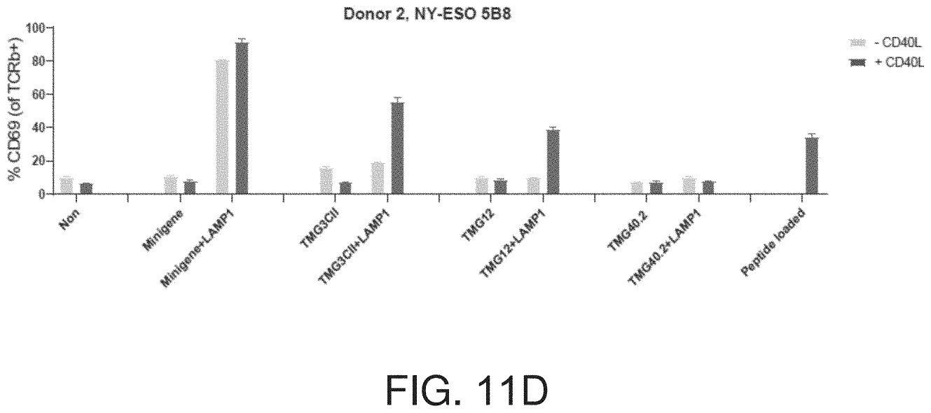

TCR expressing Jurkat T cells specific for two NY-ESO-1 derived T cell epitopes (5B8 epitope SLLMWITQCFLPVF; SEQ ID NO: 3; MHC-class II restricted T cell epitope and 1G4 epitope SLLMWITQC; SEQ ID NO: 4; MHC-class I restricted T cell epitope) are generated as described in Examples 5 and 10. 48 hours prior to co-culture with immortalized B cells, the TCR transduced Jurkat T cells are seeded at a low density (1e5/mL). Subsequently, B cells and TCR transduced Jurkat cells are cultured in a 1:1 E:T ratio (200.000 cells in 200 ul culture media per well in a 96-well microtiter plate) for 20 hours. As a positive control, B cells are loaded with either 9mer peptides (MHC-class I epitopes) by incubation with 10 ug/mL peptide or 25mer peptides (MHC-class II epitopes) by incubation with 20 ug/mL peptide overnight at 37 C. Peptide-loaded B cells are washed prior to co-culture with TCR transduced Jurkat T cells. After co-culture, CD69 expression by TCR transduced Jurkat T cells is measured by flow cytometry. The data demonstrates that CD40L transgene expression by B cells can increase MHC-class I antigen-presentation (FIG. 11A, B) as well as MHC-class II antigen presentation (FIG. 11C, D) by BCL-6/BCL-xL immortalized B cells. Furthermore, the data shows that MHC-class II restricted antigen presentation may be impaired if more than twelve 25mer polypeptides are encoded in a single TMG transgene (FIG. 11C, D).

Additional detail regarding the results in FIG. 11A-11D, demonstrating that addition of the CD40L transgene enhances recognition of antigen-expressing B cells by TCR transduced T cells on HLA-class I and HLA-class II alleles, is provided below. FIG. 11A is Donor 1: NY-ESO 1G4 TCR transduced T cell reactivity towards TMG transduced BCL-6/BCL-XL immortalized B cells with or without CD40L. Immortalized B cells were co-transduced with CD40L or not and subsequently transduced with a panel of TMG constructs encoding 1, 3, 12 or 40 epitopes with or without flanking LAMP1 sequences. NY-ESO 1G4 TCR transduced Jurkat cells directed against one of the epitopes presented on Class I were then co-cultured with the B cells and reactivity was assessed by flowcytometry focusing on upregulation of CD69 on T cells. FIG. 11B is from Donor 2: NY-ESO 1G4 TCR transduced T cell reactivity towards TMG transduced BCL-6/BCL-XL immortalized B cells with or without CD40L. Immortalized B cells were co-transduced with CD40L or not and subsequently transduced with a panel of TMG constructs encoding 1, 3, 12 or 40 epitopes with or without flanking LAMP1 sequences. NY-ESO 1G4 TCR transduced Jurkat cells directed against one of the epitopes presented on Class I were then co-cultured with the B cells and reactivity was assessed by flowcytometry focusing on upregulation of CD69 on T cells. FIG. 11C is from Donor 1: NY-ESO 5B8 TCR transduced T cell reactivity towards TMG transduced BCL-6/BCL-XL immortalized B cells with or without CD40L. Immortalized B cells were co-transduced with CD40L or not and subsequently transduced with a panel of TMG constructs encoding 1, 3, 12 or 40 epitopes with or without flanking LAMP1 sequences. NY-ESO 5B8 TCR transduced Jurkat cells directed against one of the epitopes presented on Class II were then co-cultured with the B cells and reactivity was assessed by flowcytometry focusing on upregulation of CD69 on T cells. FIG. 11D is from Donor 2: NY-ESO 5B8 TCR transduced T cell reactivity towards TMG transduced BCL-6/BCL-XL immortalized B cells with or without CD40L. Immortalized B cells were co-transduced with CD40L or not and subsequently transduced with a panel of TMG constructs encoding 1, 3, 12 or 40 epitopes with or without flanking LAMP1 sequences. NY-ESO 5B8 TCR transduced Jurkat cells directed against one of the epitopes presented on Class II were then co-cultured with the B cells and reactivity was assessed by flowcytometry focusing on upregulation of CD69 on T cells.

### Example 8: Demonstration that BCL-6/BCL-XL Immortalized B Cells can be Generated from Limited Amounts of Patient B Cells and can be Utilized to Detect Neo-Antigen Specific T Cells within Autologous TIL of a Renal Cell Cancer Patient

Example 8 demonstrates that BCL-6/BCL-xL immortalized B cells expressing a CD40L transgene and a TMG-transgene are suitable to discover antigen-specific T cell responses among tumor infiltrating lymphocytes (TIL).

Detection of antigen-specific T cells among TIL has been previously described using antigen-presenting cells, including dendritic cells and B cells (e.g. Robbins et al. Nat Med 2013 and Linnemann et al. Nat Med 2014). However, antigen-specific T cells among TIL are often present at very low frequency. This example shows that engineered immortalized B cells as described herein can be generated from small numbers of patient-derived B cells and that the generated B cells can detect low-frequency, antigen-specific T cells among TIL.

This observation further emphasizes the suitability of the antigen-presenting cells described in this disclosure to study T cell and TCR specificity.

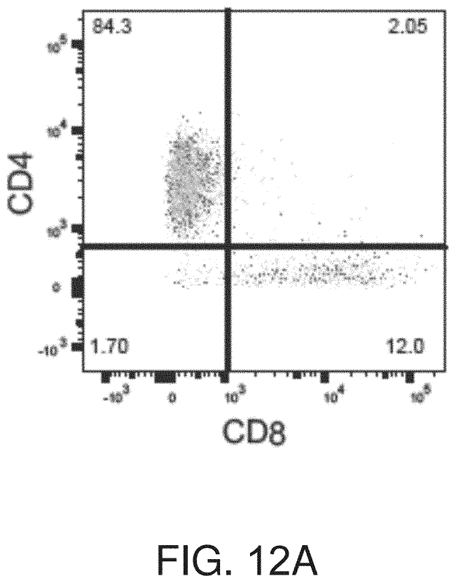

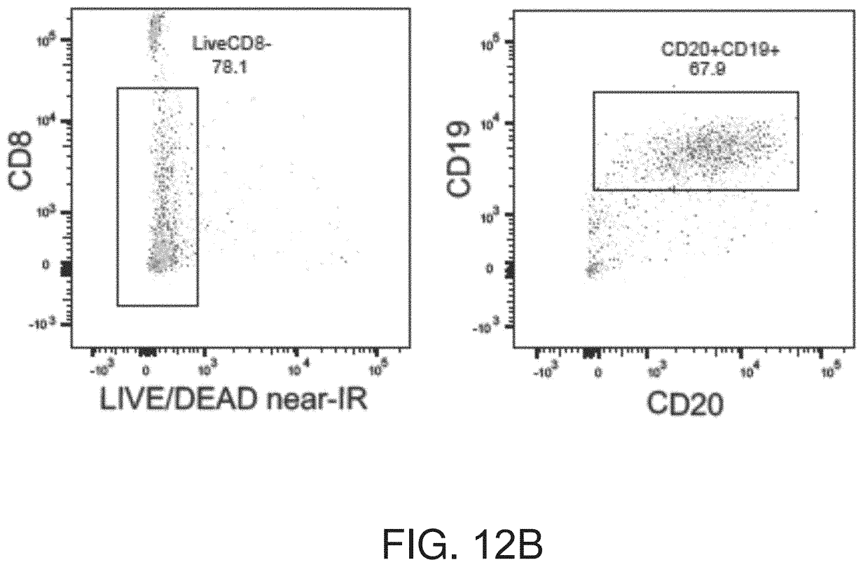

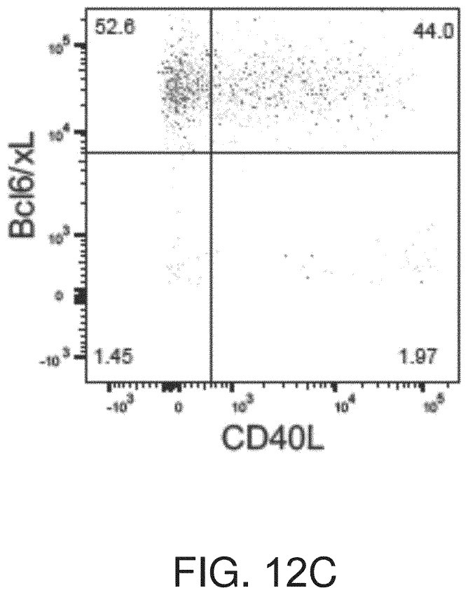

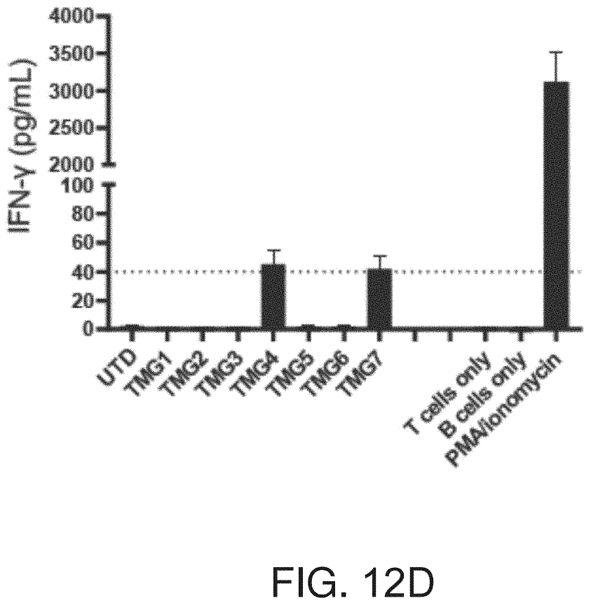

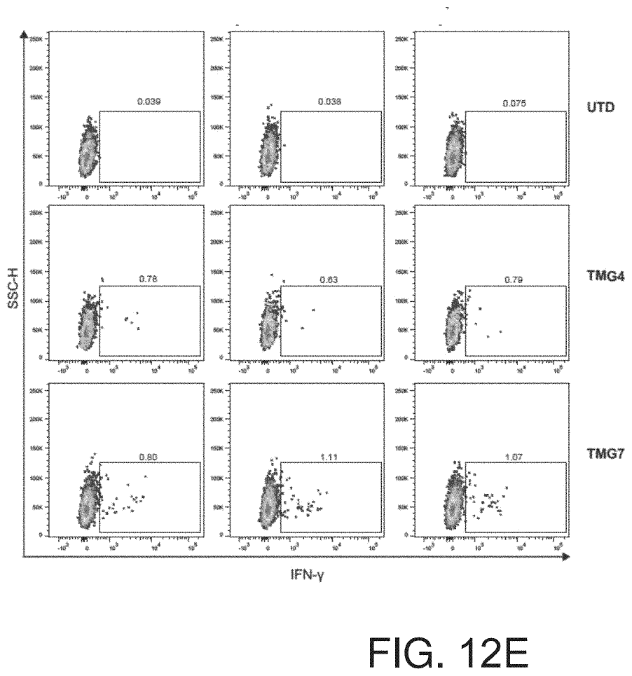

In order to analyze TIL obtained from a patient with renal cell carcinoma for the presence of neo-antigen specific T cells, TIL is expanded using rapid expansion protocol (REP) as described in the literature (Jin et al. J Immunother 20212) and known by the skilled artisan. In short, TIL are cultured in the presence of irradiated PBMCs and in the presence of 6000 IU/ml rh-IL-2 in suitable culture media (X-VIVO medium supplemented with 10% (v/v) Human Serum (HS) and 1% (v/v) Penicillin/Streptomycin (P/S)) at a ratio of TIL:feeders of 1:200. After 4 and after 8 days, half of the medium is replaced with fresh culture media supplemented with rh-IL2 (3000 IU/mL). After 11 days of REP, the CD4/CD8 composition of the cell product is monitored by flow cytometry (FIG. 12A).

Simultaneously, autologous B cells are isolated from PBMC material by negative selection as described in this invention (Example 1). For the Example 1e5 B cells are obtained from 3.1 e6 PBMC and immortalized by transduction with BCL-6/BCL-xL and CD40L with methods described in Example 10 and known to the skilled artisan. 96 hours post-transduction, expression of BCL-6/BCL-xL (as measured by GFP expression) and CD40L by live, CD8⁻, CD19⁺/CD20⁺ B cells is measured by flow cytometry (FIG. 12B, C).

Subsequently, B cells are cultured and expanded to sufficient numbers using methods described herein (Example 10). Tumor mutations are identified using whole-exome-sequencing and methods known to the skilled artisan. Subsequently, B cells are transduced with seven TMG constructs each encoding 12 different patient-specific neo-antigens flanked by LAMP1 signaling and transmembrane sequences and linked to a puromycin resistance gene and the Ly6G marker. TMG-expressing B cells are generated by retroviral transduction and enriched by antibiotic selection for 5 days as described in Example 10.

One day prior to co-culture with TMG-expressing B cells, thawed TIL are rested at 37° C. overnight. Subsequently TIL are incubated with the autologous-TMG expressing BCL-6/BCL-xL/CD40L expressing B cells in a 1:1 E:T ratio (200,000 cells in 200 ul culture media per well in a 96-well microtiter plate) at 37° C. for 20 hours. Following co-incubation, culture supernatant is harvested and analyzed for presence of IFN-γ by cytometric bead array according to manufacturer's protocol (BD). T cell responses specific for two different TMG-constructs (TMG4 and TMG7) are detected (FIG. 12D). Subsequently, detected T cell responses are confirmed by intracellular cytokine staining. Following a co-culture of TIL and TMG-expressing B cells, TIL are stained for intracellular IFN-γ using protocols known to the skilled artisan. Thereby, the presence of TMG-specific T cell among TIL is confirmed (FIG. 12E).

Additional detail regarding FIG. 12, which demonstrates that BCL-6/BCL-XL immortalized B cells can be generated from limited amounts of blood-derived B cells and used to detect neo-antigen specific T cells within autologous TIL of a renal cell cancer patient, is provided below. FIG. 12A depicts an analysis of TIL material after rapid expansion. Tumor infiltrating lymphocytes were obtained and cryopreserved after which a REP was performed to expand these cells. Analysis by flowcytometry shows a high percentage of CD4 T cells in this culture. FIG. 12B shows a selection of B cells from autologous PBMCs. B cells were isolated using magnetic bead-based negative selection. After selection B cells were immortalized and analyzed for purity of the culture by flowcytometry using antibodies directed against CD8, CD19 and CD20. Dead cells were excluded by a near-IR dye. FIG. 12C shows the transduction of selected B cells with immortalization constructs and the CD40L transgene. Selected B cells were retrovirally transduced with BCL-6/BCL-XL GFP constructs and the CD40L transgene. Transduction efficiency was assessed by flowcytometry looking at the GFP and CD154 expression of the B cells shown in 12B. FIG. 12D shows TIL reactivity as measured by the IFNγ cytokine towards 2 different TMG constructs. TMG transduced immortalized B cells were co-cultured with autologous TIL and cultures were analyzed for IFNg production in the cell medium using cytometric bead array. Specific T cell responses were found towards 2 TMG constructs. FIG. 12E shows that reactivity of TIL can also be detected on a cellular level. Cells from the cultures of 12D were analyzed by IFNg antibody staining to determine the response per cell. Columns represent multiple plots of the same condition.

### Example 9: Demonstration that CD40L Transgene Expression by BCL-6/BCL-xL Immortalized B Cells can Enhance MHC-Class II Restricted Antigen Presentation and that TMG Format can Impact the Efficacy of MHC-Class II Antigen Presentation

Example 9 demonstrates that BCL-6/BCL-xL immortalized B cells expressing a CD40L transgene show enhanced antigen-expression through both MHC-class I and II presentation pathways. The example further shows that the position of a T cell epitope and/or the nature of neighboring T cell epitopes can influence MHC-class II antigen presentation in some selected cases. This example provides further rationale to investigate additional improvements to the TMG design, including but not limited to, assessing the possibility of using TMG-designs encoding less T cell epitopes and bicistronic TMG designs encoding the same set of T cell epitopes in two different TMG-designs (varying positions and neighboring sequences).

B cells from a healthy donor are isolated and subsequently immortalized by transduction with either BCL-6/BCL-XL alone or in combination with a CD40L transgene as previously described (Example 10). Briefly, BCL-6/BCL-XL and CD40L constructs are separately transfected into 293Vec-Baev (BioVec Pharma) or Phoenix-Ampho (ATCC) virus producer cells using Fugene transfection reagent and protocols known to the skilled artisan. The resulting retroviral virions are used to transduce immortalized B cells isolated from a healthy donor (HC858) with BCL-6/BCL-XL alone or in combination with CD40L. This donor expresses the following HLA-alleles: HLA-A*02:01, DPB1*04:01, DRB1*15:01 and DQB1*06:02.

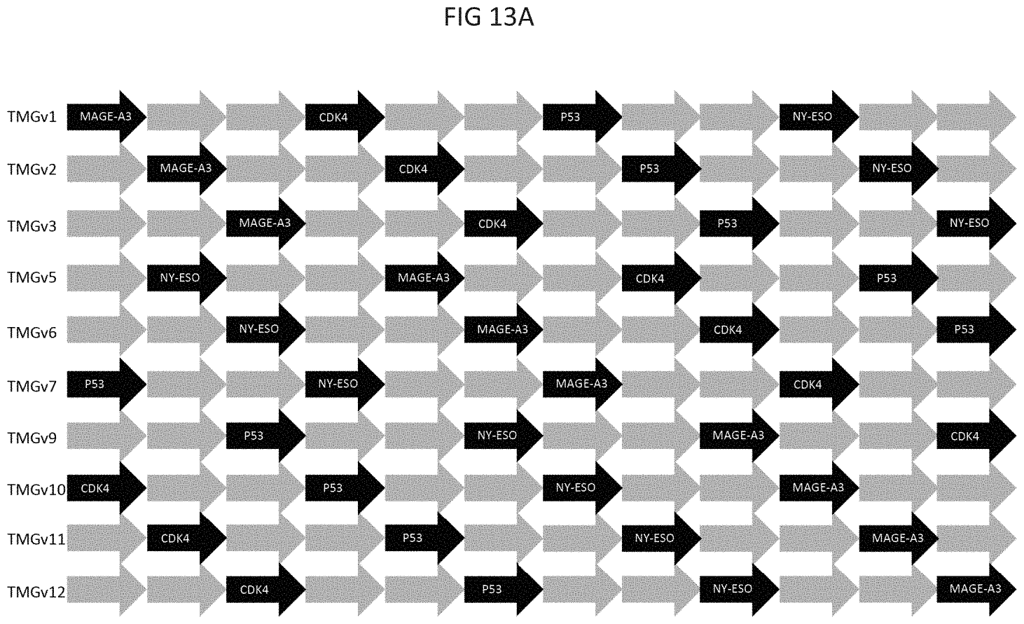

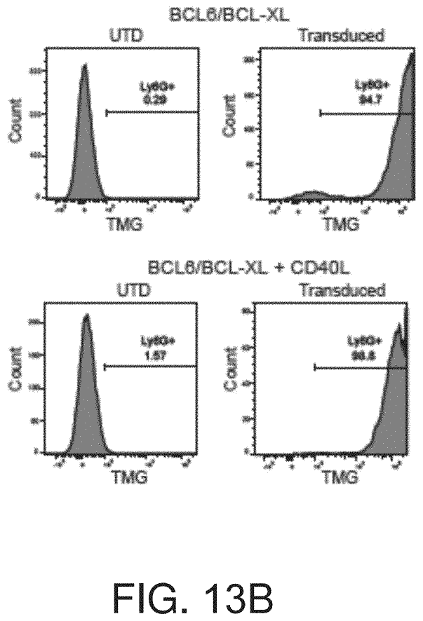

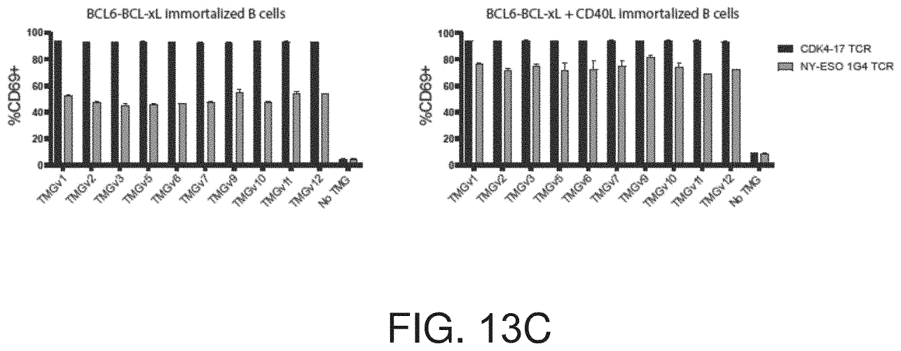

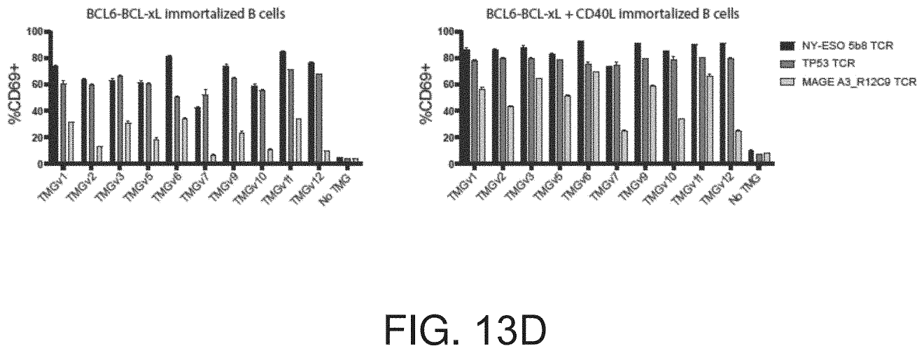

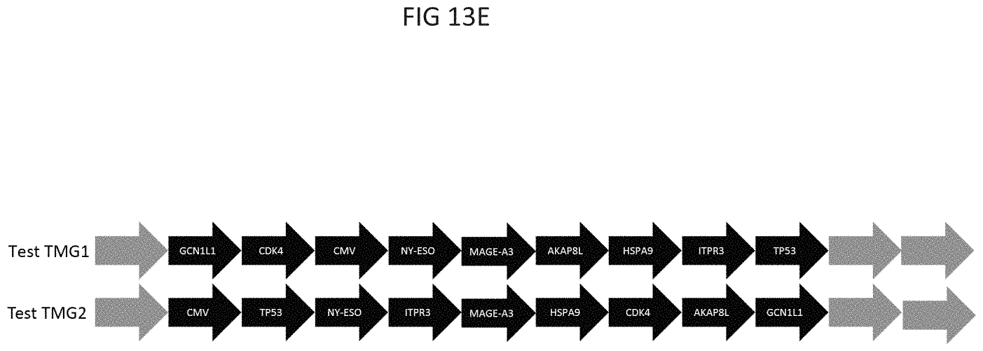

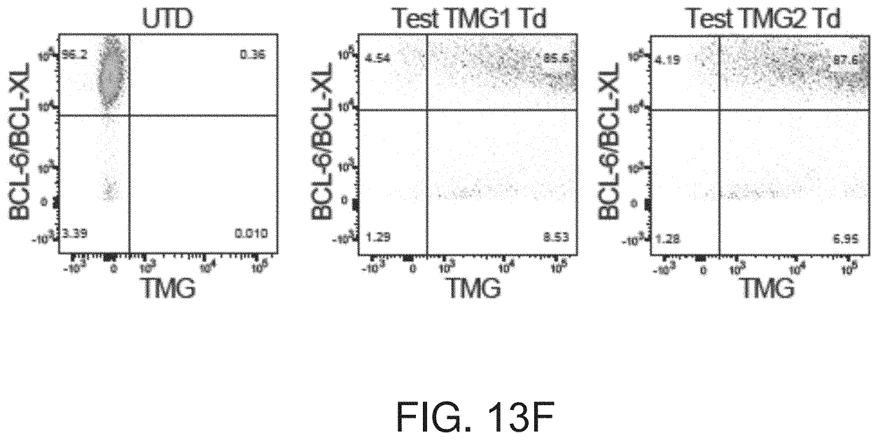

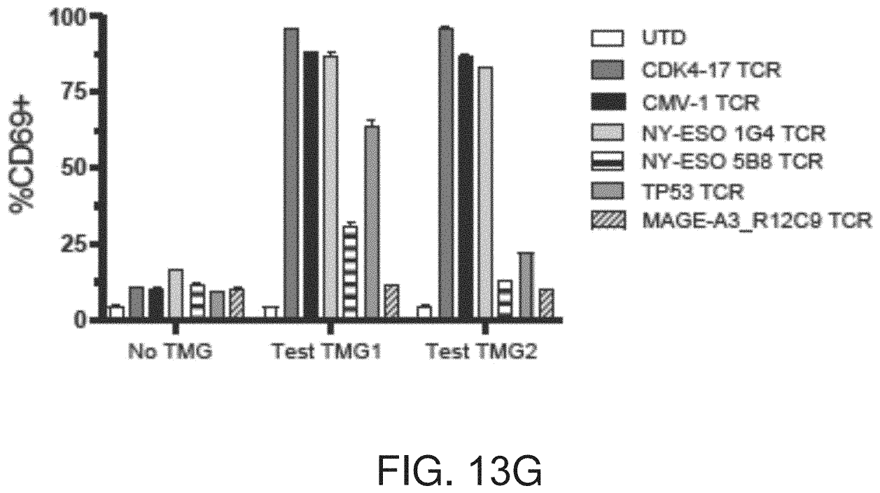

Various Tandem-minigene (TMG) including 12 different 25mer polypeptides LAMP-1 signaling and transmembrane domain formats are designed (FIG. 13A). Each TMG includes four known epitopes are placed within the TMG construct at different positions, namely CDK4, NY-ESO-1, TP53 and MAGE-A3/A6. Other positions are filled with additional irrelevant 25mer-polypeptide sequences derived from single amino acid mutations detected in selected unrelated human tumors. Between different TMGs, the position of individual 25mer polypeptide sequences is varied while neighboring sequences are kept constant (FIG. 13A), thereby allowing one to assess whether the position of certain epitopes can have an influence on the recognition by MHC class II restricted TCRs independent of neighboring sequences. Separately, two additional Tandem-minigene (TMG) formats were designed and 9 known epitopes were placed within the two TMG constructs at different positions, except for the MAGE-A3/A6 epitope which is placed at position six in both TMG designs (FIG. 13E). The known epitopes are CDK4, NY-ESO-1, TP53 and MAGE-A3/A6, GCN1L1, AKAP8L, ITPR3, CMV, HSPA9. Three additional sequences for 25mer polypeptides are added in order to create a TMG with a total of 12 encoded polypeptides.

In order to generate TMG-expressing B cells, constructs encoding TMGs were transfected into 293Vec-Baev virus producer cells (BioVec Pharma) using Fugene transfection reagent and protocols known to the skilled artisan. The resulting retroviral virions were used to transduce immortalized B cells. As described herein (Example 10), TMG-expressing B cells can be detected and enriched using a Ly6G marker and Puromycin resistance gene encoded in the TMG. Antibiotic selection of genetically modified cells is known to an ordinary person skilled in the art and described herein (Example 10). Expression of the TMG transgene is detected by flow cytometry using a Ly6G specific antibody (FIG. 13B, F).

Jurkat T cells expressing TCRs recognizing either one of the MHC class I restricted T cell epitopes (CDK4-17, NY-ESO-1, CMV-1) or MHC class II restricted T cell epitopes (NY-ESO-1, MAGE-A3/A6 and TP53 can be used to evaluate presentation of T cell epitopes by TMG-expressing B cells. In order to generate TCR transduced Jurkat T cells, TCR-encoding plasmids are transfected into 293Vec-Baev virus producer cells (BioVec Pharma) or Phoenix-Ampho (ATCC) using Fugene transfection reagent and protocols known to the skilled artisan. The resulting retroviral virions are used to transduce a Jurkat reporter T cell line. The Jurkat reporter T cell line lacks endogenous TCR expression (generation of such a genetic knock-out being described for example in Mezzadra et al Nature 2017) and is modified to express human CD8α and CD8β after transduction with a CD8α-P2A-CD8β transgene (SEQ ID NO: 2) using methods known to the skilled artisan. The use of murine TCR constant domain sequences as incorporated in the TCR transgene allows for the detection of TCR-modified Jurkat T cells by flow cytometry using a murine TCRβ constant domain specific antibody. Most of the TCR-modified T cells are sorted to get a purity >95%. The NY-ESO-1 TCR is transduced into a Jurkat T cell line that is only modified to express human CD8a and CD8β after transduction with a CD8α-P2A-CD8β transgene (SEQ ID NO: 2) using methods known to the skilled artisan and is not sorted. The TP53-TCR construct is linked to a Blasticidin transgene that can be used as selection marker. TP53-TCR-modified T cells are selected with 6 ug/mL Blasticidin at a cell density of 0.25e6/mL cells for 96 hours. The medium is washed off and the cells are cultured medium at a cell density of 0.25e6/mL cells in the absence of Blasticidin for another 72 hours to complete the selection. On the day of the assay, >70% of the cells is shown to be TCR+.

Subsequently, TCR transduced Jurkat T cells are stimulated with APCs that were engineered to present the different TMG formats. 48 hours prior to the co-culture Jurkat reporter T cells were seeded at a low density (0.1×10⁶ cells per ml). B cells are cultured as previously described, at a density of 0.2-0.25e6 cells/mL in the presence of rh-IL-21 (50 ng/ml). TMG-expressing B cells and TCR transduced Jurkat T cells are co-cultured in a 1:1 E:T ratio (200.000 cells total in 200 ul per well in a U-bottom TC-treated 96-well plate). Plates are centrifuged at 1000 rpm for 1 minute, and incubated for 20-22 hours at 37° C. After 20 hours, cells are stained with antibodies directed against CD62L (data not shown) and CD69 and specific recognition by TCR td T cells is measured by CD69 upregulation on a flow cytometer (FIG. 13C; FIG. 13D; FIG. 13G; data for CD62L is not shown). The presented data shows that CD40L expression by TMG-expressing B cells can improve both MHC-class I and MHC-class II restricted antigen presentation by B cells. In some instances, it appears that the position of a T cell epitope and/or the nature of neighboring T cell epitopes can influence MHC-class II antigen presentation. A further possible interpretation is that overall stability of the TMG/polypeptides and/or targeting of the TMG to the cell membrane may be enhanced. Overall, this indicates that further improvements can be made to the design of the TMG-transgene cassette and the delivery of the TMG into the B cells.

Additional details regarding the results in FIGS. 13A-13G are provided below. FIG. 13A provides a Schematic overview of various TMG formats. Each TMG includes four known epitopes that are placed within the TMG construct at different positions, namely CDK4, NY-ESO-1, TP53 and MAGE-A3/A6. Other positions are filled with additional 25mer-polypeptide sequences derived from randomly selected single amino acid changes detected in human tumors. Between different TMGs, the position of individual 25mer polypeptide sequences is varied while neighboring sequences are kept constant. FIG. 13B depicts the stable transduction of a TMG on BCL-6/BCL-XL and BCL-6/BCL-XL-CD40L immortalized B cells. The bcl-6-bcl-xL construct was transfected into 293Vec-Baev cells for virus production, and the resulting viral supernatant was used for retroviral transduction of immortalized B cells. Expression of TMG was measured by FACS using an antibody directed against Ly6G. Gating strategy: Lymphocytes>live cells>CD20+ cells>GFP+ cells>Ly6G. FIG. 13C shows Class-I restricted T cell reactivity towards BCL-6/BCL-XL and BCL-6/BCL-XL-CD40L immortalized B cells expressing various TMG formats. Immortalized B cells from a healthy donor were transduced separately with various TMG constructs from 13A. TCR transduced Jurkat T cells were co-cultured with the TMG-expressing B cells overnight and subsequently analyzed for upregulation of CD69 by flowcytometry. FIG. 13D shows Class-II restricted T cell reactivity towards BCL-6/BCL-XL and BCL-6/BCL-XL-CD40L immortalized B cells expressing various TMG formats. Immortalized B cells from a healthy donor were transduced with various TMG constructs from A. TCR transduced Jurkat T cells were co-cultured with the TMG-expressing B cells overnight and subsequently analyzed for upregulation of CD69 by flowcytometry. FIG. 13E is a Schematic overview of 2 additional TMG formats. Nine known epitopes were placed within the two TMG constructs at different positions, except for the MAGE-A3/A6 epitope which is placed at position six in both TMG designs. The known epitopes are CDK4, NY-ESO-1, TP53 and MAGE-A3/A6, GCN1L1, AKAP8L, ITPR3, CMV, HSPA9. Three additional sequences for 25mer polypeptides are added in order to create a TMG with a total of 12 encoded polypeptides. FIG. 13F shows stable transduction of TMG on BCL-6/BCL-XL-CD40L immortalized B cells. The TMG construct was transfected into 293Vec-Baev cells for virus production, and the resulting viral supernatant was used for retroviral transduction of immortalized B cells. Expression of TMG was measured by FACS using an antibody directed against Ly6G. Gating strategy: Lymphocytes>single cells>live cells. FIG. 13G shows Class I- and class II-restricted T cell reactivity towards BCL-6/BCL-XL-CD40L immortalized B cells expressing 2 different TMG formats. Immortalized B cells from a healthy donor were transduced with various TMG constructs from FIG. 13E. TCR transduced Jurkat T cells were co-cultured with the TMG-expressing B cells overnight and subsequently analyzed for upregulation of CD69 by flowcytometry.

### Example 10: Detailed Description of B Cell Immortalization, B Cell Transduction and Generation of TCR Td Jurkat T Cells

### B Cell Isolation

B cells are isolated from cryopreserved healthy donor or patient PBMC using one of the available magnetic bead cell separation techniques known to the skilled artisan. Magnetic bead-based cell separation techniques used in this invention include: (i) isolation of B cells by negative selection. For example, B cells are isolated using the MojoSort Human Pan B cell Isolation Kit (Biolegend) according to manufacturer's protocol. Briefly, PMBCs are incubated with biotin-labelled antibodies against CD2, CD3, CD14, CD16, CD16, CD36, CD56, CD123 and CD235. After incubation, magnetic streptavidin labelled nanobeads are added. After the second incubation, the tube containing the cells is put inside a magnet and the magnetically labelled fraction is retained on the sides of the tube and the liquid will contain the untouched B cells and can be aspirated from the tube for further use. (FIG. 7A). (ii) isolation of B cells by positive selection. For example, B cells are isolated using Miltenyi anti-CD20 microbeads and a Miltenyi AutoMACS instrument according to manufacturer's instruction. Briefly, depending on the viability of the PBMC a dead cell removal kit (Miltenyi) can be used to remove the dead cells from the sample prior to continuing to B cell isolation. Removal of dead cells can improve the cell separation due to less aspecific binding of microbeads to dead cells. A viability of <85% live cells is in general considered a poor viability and removal of dead cells prior to cell separation is desired. Either after dead cell removal, or directly from thawed PBMC, B cells are isolated by incubating PBMC with anti-CD20 microbeads. After a 15-minute incubation step the sample is loaded onto the AutoMACS (Miltenyi) and a positive selection method can be run on the machine, e.g. Possel or Possel sensitive (PosselS). The positive fraction contains the CD20⁺ B cells which can be used for downstream processing, such as immortalization of B cells. The negative fraction contains all other cells, including T cells. One can use this fraction of cells to isolate certain subtypes of T cells, for example antigen-experienced T cells by using a Miltenyi Pan T cell isolation Kit. Simultaneous isolation of B and T cells from PMBC is of interest if both cell subsets are used in down-stream processes, such evaluation of T cell responses or isolation of TCR genes from a sample using autologous B cells as antigen-presenting cells.

An ordinary person skilled in the art will appreciate that primary human B cells may be isolated by using alternative isolation methods, such as flow cytometry sorting and that any B cell isolation method should not interfere with downstream processing of isolated B cells, including but not limited to interference with EBV infection or retroviral transduction BCL-6/BCL-xL transgenes.

B Cell Immortalization with BCL-6/BCL-XL

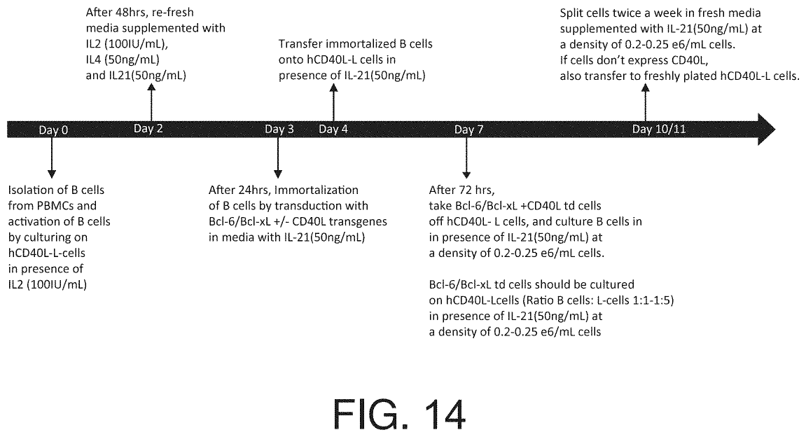

BCL-6/BCL-xL genes are expressed in germinal center B cells and allow them to proliferate without differentiating. CD40 receptor stimulation on B cells is useful to maintain primary human B cells in vitro. Kwakkenbos et al. described a method to immortalize B cells by introducing BCL-6/BCL-xL transgenes into primary B cells and culture these cells in the presence of IL-21 and CD40L to stimulate the cells and expand them for a long period of time (Kwakkenbos et al. Nat Med 2010). A general timeline for immortalization of primary human B cells based on introduction of BCL-6/BCL-xL transgenes is described in FIG. 14.

In order to generate BCL-6/BCL-xL immortalized B cell in this invention, isolated B cells are activated by seeding hCD40L-expressing L-cells in a TC-treated plate at least 4 hours before addition of B cells. CD40L expressing L cells are generated by retrovirally transducing murine L cells (ATCC) with the CD40L transgene. Retrovirus is generated by transfection of Phoenix-Eco producer cells and this virus is used to transduce L cells. CD40L transduced cells are subsequently selected by FACS-based sorting and irradiated with 60 Gy.

Generally, between 20.000 and 200.000 isolated B cells are cultured on irradiated hCD40L-expressing L-cells at a ratio between 1:1-1:5. Typically, either TC-treated 24-well culture plates (1e⁵ L-cells and 1-5e⁵ B cells) or TC-treated 6-well culture plates (1e⁶ L-cells and 1-5e⁶ B cells) are used based on the yield of isolated B cells. B cells are cultured in Complete B cell medium (RPMI1640 medium supplemented with 10% (v/v) Fetal Calf Serum (FCS), 1% (v/v) Penicillin/Streptomycin (P/S), 1% (v/v) Sodium Pyruvate, 1% (v/v) Non-essential amino acids, 1% (v/v) Glutamax, 50 nM 2-Mercaptoethanol) in the presence of rh-IL-2 (100 IU/mL).

After 48 hours, the culture medium is replaced with fresh complete B cell medium supplemented with rh-IL-2 (100 IU/mL), rh-IL-4 (50 ng/mL) and rh-IL-21 (50 ng/mL). After 24 hours, the activated B cells are immortalized by stable transduction with a BCL-6/BCL-XL retroviral construct either alone or in combination with a CD40L transgene. Protocols for retroviral transduction of primary B cells have been described in the literature (e.g. Kwakkenbos et al. Nat Med 2010) and are known to the skilled artisan. In short, Phoenix-Ampho or 293Vec-Baev producer cells are transfected with BCL-6/BCL-XL and CD40L encoding plasmid DNA using Fugene6 transfection reagent and protocols known by the skilled artisan. Viral supernatant is harvested after 48 hours and used to transduce B cells by spin-based transduction which is known to the skilled artisan.

Post-transduction, BCL-6/BCL-xL transduced B cells are cultured in complete B cell medium supplemented with rh-IL-21 (50 ng/mL). Prolonged culturing of B cells in medium containing rh-IL-2 and IL-4 can lead to undesired outgrowth of residual T cells. After 24 hours, cells are transferred to TC-treated culture plates that contain hCD40L-expressing L-cells that have been adhered to the bottom and cells are cultured in complete B cell medium in the presence of rh-IL-21 (50 ng/mL). B cells are cultured at densities between 200.000-250.000 cells/mL and co-cultured with CD40L-expressing L-cells at ratios between 1:1-1:5. Typically, B cells are split twice a week to cell densities of 200.000-250.000 cells/mL and plated onto hCD40L-expressing L-cells at ratios between 1:1-1:5 in complete B cell medium supplemented with IL-21 (50 ng/mL).

BCL-6/BCL-xL/CD40L transduced B cells are removed from hCD40L-expressing L-cells after 72 hours and transferred into a new TC-treated tissue culture plate or tissue culture flask and cultured in complete B cell medium in the presence of rh-IL-21 (50 ng/mL) at densities of 200,000-250,000 cells/mL. Cells are split twice a week to maintain cell densities and supply fresh rh-IL-21.

B Cell Immortalization by Infection with Epstein-Barr Virus (EBV)

Another immortalization method is based on infection of the B cells by EBV, for example by Traggiai et al. Meth Mol Biol 2021.T. The B cells are isolated as described above and are cultured in complete B cell medium (RPMI1640 medium supplemented with 10% (v/v) Fetal Calf Serum (FCS), 1% (v/v) Penicillin/Streptomycin (P/S), 1% (v/v) Sodium Pyruvate, 1% (v/v) Non-essential amino acids, 1% (v/v) Glutamax, 50 nM 2-Mercaptoethanol) supplemented with 2×CpG ODN 2006 (final concentration of 2.5 ug/mL) to activate the Toll Like Receptors for subsequent EBV infection with 1 mL EBV-virus supernatant (ATCC) and the cells are incubated for 4 hours at 37 C. Cells are washed and resuspended in complete B cell medium supplemented with 2×CpG ODN 2006 (final concentration of 2.5 ug/mL) and 1% EBV virus supernatant. On day 3 and day 8, when the medium turns yellow, cells are washed and resuspended in complete B cell medium without CpG. Once proliferation is visible by the presence of clumps of B cells, the cells are washed and cultured in RPMI supplemented with 20% (v/v) FCS and 1% (v/v) P/S. Every 2 or 3 days the cells are split in a ratio of 1:2

Transduction of B Cells with Tandem-Minigene (TMG) Transgenes

The generation of antigen-expressing, immortalized B cells using different immortalization techniques and stable transduction with transgenes encoding patient-specific mutations in a tandem-minigene format (TMG) can be of interest for TCR discovery. TMG-transgenes can be designed in different formats. For example, in addition to variable number of putative T cell epitopes, TMG-encoding gene cassettes can additionally contain both a Ly6G protein as well as a puromycin-resistance gene. This allows to select TMG-expressing B cells by antibiotic selection and detect TMG-expression by flow cytometry using a Ly6G-specific antibody.

In order to retrovirally transduce BCL-6/BCL-XL immortalized B cells with TMG-transgenes, Phoenix-Ampho or 293Vec-Baev producer cells are transfected with TMG-encoding plasmid DNA using Fugene6 transfection reagent and protocols known by the skilled artisan. Viral supernatant is harvested after 48 hours and used to transduce immortalized B cells by spin-based transduction which is known to a skilled artisan. The next day, cells are transferred to a fresh TC-treated culture plate and fresh complete B cell medium supplemented with rh-IL-21 (50 ng/mL) is added to the cells in a 1:1 volume.

After 72-hours, half of the medium is removed, and fresh complete B cell medium supplemented with IL-21 (50 ng/mL) and Puromycin (1 ug/mL) is added to the cells in a 1:1 volume. Antibiotic selection will continue for four days (unless otherwise indicated), after which the frequency of TMG-expressing B cells after puromycin-based selection is measured by flow cytometry using a fluorochrome labeled Ly6G-specific antibody.

Notably, EBV-immortalized B cells can likewise be transduced with TMG-transgenes. Phoenix-Ampho or 293Vec-Baev producer cells are transfected with TMG-encoding plasmid DNA using Fugene6 transfection reagent and protocols known by the skilled artisan. Viral supernatant is harvested after 48 hours and used to transduce immortalized B cells by spin-based transduction which is known to a skilled artisan. The next day, fresh medium (RPMI1640, 20% (v/v) FCS, 1% (v/v) Penicillin and Streptomycin) is added to the cells in a 1:1 volume.

After 72-hours, half of the medium is removed, and fresh medium supplemented with Puromycin (5 ug/mL) is added to the cells in a 1:1 volume. Antibiotic selection will continue for four days (unless otherwise indicated), after which the frequency of TMG-expressing B cells after puromycin-based selection is measured by flow cytometry using a fluorochrome labeled Ly6G-specific antibody.

Transduction of Jurkat T Cells with TCR-Genes

Jurkat T cells expressing TCRs recognizing either MHC class I restricted T cell epitopes or MHC class II restricted T cell epitopes can be used to evaluate presentation of T cell epitopes by TMG-expressing B cells. In order to generate TCR transduced Jurkat T cells, TCR-encoding plasmids are transfected into Phoenix-Ampho (ATCC) or 293Vec-Baev (BioVec Pharma) virus producer cells using Fugene transfection reagent and protocols known to the skilled artisan. The resulting retroviral virions are used to transduce a Jurkat reporter T cell line. The artisan skilled in the art can choose from a variety of different Jurkat reporter T cell lines. (i) The Jurkat reporter T cell line lacks endogenous TCR expression (generation of such a genetic knock-out being described for example in Mezzadra et al Nature 2017) and is modified to express human CD8α and CD8β after transduction with a CD8α-P2A-CD8β transgene using methods known to the skilled artisan. (ii) The Jurkat reporter T cell line is modified to express human CD8α and CD8β after transduction with a CD8α-P2A-CD8β transgene (SEQ ID NO: 2) using methods known to the skilled artisan. (iii) The Jurkat reporter T cell line lacks endogenous TCR expression (generation of such a genetic knock-out being described for example in Mezzadra et al Nature 2017) and is modified to express human CD4, CD8α and CD8β after transduction with a CD8α-P2A-CD8β-T2A-CD4 transgene using methods known to the skilled artisan. The latter one (iii) is preferred as this cell line has no endogenous TCR and maximal expression of CD4 and CD8 on its cell surface which allows expression of both MHC-I and MHC-II restricted TCRs. The use of murine TCR constant domain sequences as incorporated in the TCR transgene allows for the detection of TCR-modified Jurkat T cells by flow cytometry using a murine TCRβ constant domain specific antibody. TCR-modified Jurkat T cells can be enriched by either flow-based sorting or by antibiotic selection if there is a (i) puromycin or (ii) blasticidin-selection marker transgene linked to the TCR construct. (i) Briefly, TCR-modified T cells are cultured at 0.5e6/mL cells in the presence of 6 ug/mL blasticidin for 96 hours. The medium is washed off and the cells are cultured at 0.5e6/mL cells in the absence of blasticidin for another 72 hours to complete the selection In some situations, TCR-modified T cells are cultured at 0.25e6/mL cells in the presence of 6 ug/mL blasticidin for 96 hours. The medium is washed off and the cells are cultured at 0.25e6/mL cells in the absence of blasticidin for another 72 hours to complete the selection. (ii) Briefly, TCR-modified T cells are cultured at 0.5e6/mL cells in the presence of 250 ng/mL puromycin for 96 hours. Jurkat T cells are cultured in medium (RPMI1640 medium supplemented with 20% (v/v) FCS, 1% (v/v) Penicillin and Streptomycin) at densities between 0.1-1e6/mL cells.

### Example 11: Efficient Presentation of T Cell Epitopes on MHC Class I Complexes Using Electroporation of Long Peptides

Immortalized primary human B cells that are electroporated with a polypeptide of a uniform length (25mer) will be efficiently processed and present T cell epitopes encoded within the polypeptide via the MHC class I pathway and activate T cells expressing an MHC class I restricted TCR specific for the T cell epitope encoded within the polypeptide. BCL-6/BCL-xL-CD40L immortalized B cells from a healthy donor were electroporated with short (10mer) (ALLETPSLLL, SEQ ID NO: 5) or long (25mer) (FSWNVKAALLETPSLLLAKVGIALK, SEQ ID NO: 6) peptides encoding the GCN1L1 mutated peptide or CMV mutated long peptide (WQAGILARNLVPMVATVQGQNLKYQ, SEQ ID NO: 7), using a method known to the skilled artisan. Briefly, for each sample, 0.2e6 B cells were harvested and washed with PBS. After removal of all PBS, the dry pellet was resuspended in electroporation buffer (SF-buffer, Lonza) at a concentration of 25e6 cells per mL. Of this mixture, 20 μL per sample was added to a 96 well tissue-culture-plate that contains the appropriate amount of peptide resulting in the indicated peptide concentration. Cells were transferred to a 16-well electroporation strip (Lonza) and subsequently electroporated using the DN100 program (Lonza Amaxa). Post-electroporation, cells were rested for 10 minutes at 37° C. and transferred to a 96 well round bottom tissue-culture plate in a final volume of 200 μL of Complete B cell Medium (RPMI1640 medium supplemented with 10% (v/v) Fetal Calf Serum (FCS), 1% (v/v) Penicillin/Streptomycin (P/S), 1% (v/v) Sodium Pyruvate, 1% (v/v) Non-essential amino acids, 1% (v/v) Glutamax, 50 nM 2-Mercaptoethanol) and incubated for 0, 2, 4, 5, 6, 10 or 24 hrs at 37° C. The resting period post peptide electroporation allows for exogenous loading of the peptide onto the target cells in a parallel experimental condition. After this incubation, cells were harvested and 1e5 cells per well were plated before the cells were washed extensively to remove any residual peptide. As a positive control, BCL-6/BCL-xL-CD40L immortalized B cells expressing a TMG in which the mutated epitopes of GCN1L1, CMV and MAGE-A3 (TMG12L1, MAGE-A3 related results are presented in Example 12) are encoded were included as controls in this experiment. In order to generate TCR transduced Jurkat T cells, TCR-encoding plasmids were transfected into Phoenix-Ampho virus producer cells (ATCC) using Fugene transfection reagent and protocols known to the skilled artisan. The resulting retroviral virions were used to transduce a Jurkat reporter T cell line. The Jurkat reporter T cell line lacks endogenous TCR expression (generation of such a genetic knock-out being described for example in Mezzadra et al Nature 2017) and was modified to express human CD4, CD8α and CD8β after transduction with a CD8α-P2A-CD8β-T2A-CD4 transgene using methods known to the skilled artisan. The use of murine TCR constant domain sequences as incorporated in the TCR transgene allows for the detection of TCR-modified Jurkat T cells by flow cytometry using a murine TCRβ constant domain specific antibody. Effector TCR-KO Jurkat T cells expressing the GCN1L1 TCR or the CMV1 TCR were seeded at 1e5 cells/mL 2 days prior to the experiment to reduce any background of the activation marker. The peptide electroporated and TMG-expressing B cells were co-cultured in a 1:1 E:T ratio with TCR transduced Jurkat T cells (200,000 cells total in 200 μL per well in a U-bottom TC-treated 96-well plate). Plates were centrifuged at 1000 rpm for 1 minute, and incubated for 20-22 hours at 37° C. After 20 hours, cells were stained with antibodies directed against CD8 and CD69 and specific recognition by TCR td T cells was measured by CD69 upregulation on a flow cytometer.

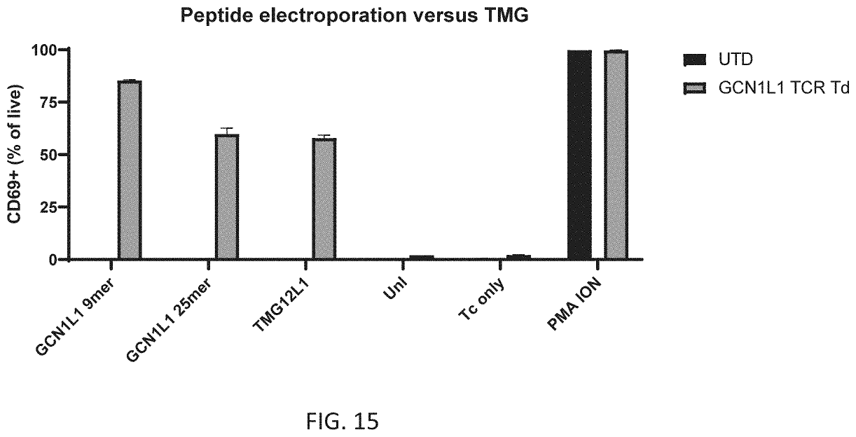

FIG. 15 shows efficient presentation of the GCN1L1 antigen from both short and long peptides after peptide electroporation. B cells expressing a TMG12L1-construct encoding the epitopes (‘TMG12L1’), the parental BCL-6/BCL-xL/CD40L immortalized B cells (‘Unl’), medium without B cells (‘Tc alone’) or medium supplemented with 50 ng/mL PMA and 1 uM Ionomycin (TMA/Ion′) were used in the co-culture as controls for antigen presentation and/or T cell activation. After a (co-)culture with GCN1L1 TCR-transduced effector cells, T cell activation was measured by CD69+ expression of the live cells.

T Cell Activation Mediated by the Class-I Restricted GCN1L1 TCR at Various Times after Peptide Electroporation or Loading.

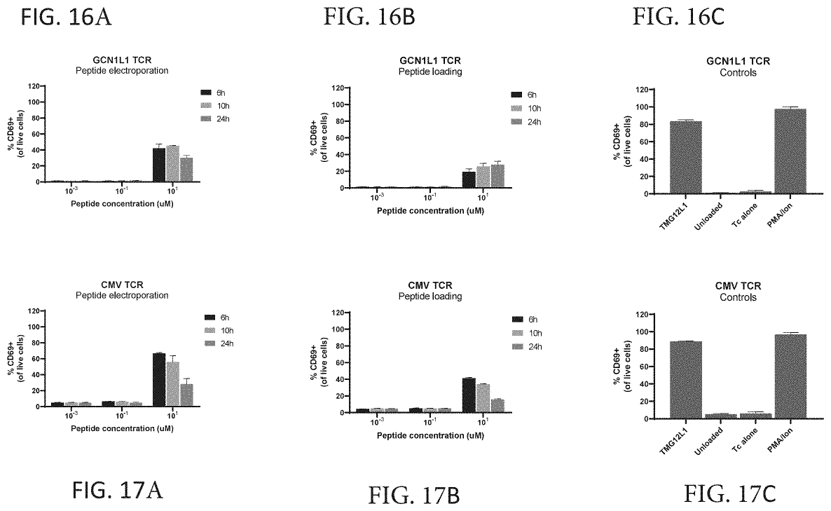

The mutated GCN1L1 epitope was electroporated or loaded onto BCL-6/BCL-xL/CD40L immortalized B cells. T cell activation was measured for various concentrations of peptide after 6, 10 or 24 hours of post-electroporation resting (FIG. 16A) or were loaded with peptide (FIG. 16B) for similar time-points. B cells expressing a TMG12L1-construct encoding the same epitope (‘TMG12L1’), the parental BCL-6/BCL-xL/CD40L immortalized B cells (‘Unloaded’), medium without B cells (‘TC alone’) or medium supplemented with 50 ng/uL PMA and 1 uM Ionomycin (‘PMA/Ion’) were used in the co-culture as controls for antigen presentation and/or T cell activation (FIG. 16C). After a (co-) culture with GCN1L1 TCR-transduced effector cells, T cell activation was measured by CD69+ expression of live cells.

T Cell Activation Mediated by the Class-I Restricted CMV TCR at Various Times after Peptide Electroporation or Loading.

A CMV epitope was electroporated or loaded onto BCL-6/BCL-xL/CD40L immortalized B cells. T cell activation was measured for various concentrations of peptide after 6, 10 or 24 hours of post-electroporation resting (FIG. 17A) or were loaded with peptide (FIG. 17B) for similar time-points. B cells expressing a TMG12L1-construct encoding the same epitope (‘TMG12L1’), the parental BCL-6/BCL-xL/CD40L immortalized B cells (‘Unloaded’), medium without B cells (‘TC alone’) or medium supplemented with 50 ng/uL PMA and 1 uM Ionomycin (TMA/Ion′) were used in the co-culture as controls for antigen presentation and/or T cell activation (FIG. 17C). After a (co-)culture with CMV TCR-transduced effector cells, T cell activation was measured by CD69+ expression of live cells.

The presented data shows that long and short peptides can be efficiently processed and presented on MHC class I molecules using peptide electroporation and shows comparable recognition by TCR transduced T cells of these target cells compared to TMG expressing target B cells (FIGS. 15-17). The data also show that 6h of processing post-electroporation shows superior recognition of electroporated B cells compared to peptide-loaded (FIGS. 16 and 17).

### Example 12: Long 25-Mer Peptides can Successfully be Processed and Presented on Both MHC-Class I and Class-II Molecules

Immortalized primary human B cells will be electroporated with a polypeptide of a uniform length (25mer) and will efficiently process and present T cell epitopes encoded within the polypeptide via the MHC class II pathway and activate T cells expressing an MHC class II restricted TCR specific for the T cell epitope encoded within the polypeptide.

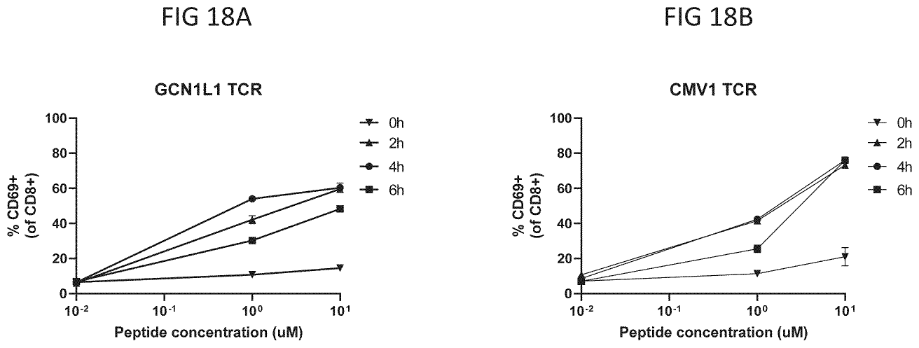

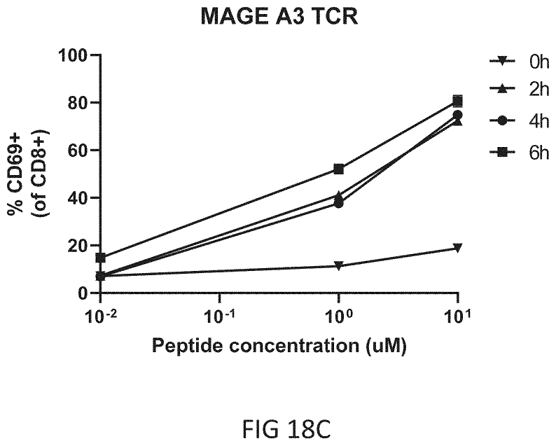

In an experiment conducted as in Example 11 (except that the peptide concentrations and post-electroporation rests are as in FIGS. 18A-18C), the GCN1L1 and CMV TCRs and a MAGEA3 TCR and corresponding mutated long peptides (GCN1L1—SEQ ID NO:6) (CMV—SEQ ID NO: 7) (MAGEA3—ILGDPKKLLTQHFVQENYLEYRQVP, SEQ ID NO: 8) were used. The data are shown in FIGS. 18A-18C. All peptides (including both MHC class I restricted GCN1L1 and CMV TCRs and the class II restricted MAGE-A3 TCR) were able to elicit a response with a post-electroporation rest of 2 hours or longer.

Taken together with Example 11, these examples show that long 25-mer peptides can successfully be processed and presented on both MHC-class I and class-II molecules using peptide electroporation.

### Example 13: Peptide Sensitivity Across a Range of Concentrations for the Tested TCRs

Immortalized primary human B cells will be electroporated with graded concentration of a polypeptide of a uniform length (25mer) encoding a T cell epitope will be used in immune-assays, involving the co-culture of these antigen expressing immortalized B cells with T cells engineered to express a defined MHC class I restricted TCR, to assess the sensitivity of the TCR. The sensitivity will then be assayed.

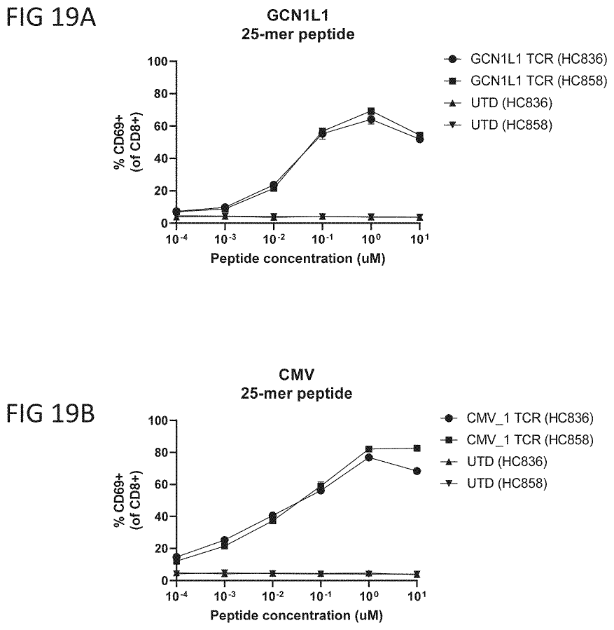

The exemplary concentration of long peptide in the electroporation assays was determined in a peptide titration experiment using immortalized B cells from two healthy donors and autologous PBMCs that are transduced with the TCR corresponding to the peptides (FIGS. 19A-19B). BCL-6/BCL-XL-CD40L immortalized B cells from healthy donors HC836 and HC858 were electroporated as described above with titrating amounts of peptide encoding the mutated GCN1L1 or viral CMV ranging from 0 uM to 100 uM. B cells are rested for 4 hours at 37° C. post-electroporation (resulting in a final concentration range of 0 uM to 10 uM). Autologous PBMCs that are transduced with the GCN1L1 or CMV TCR were added in a 1:1 effector:target cell ratio. Plates were centrifuged at 1000 rpm for 1 minute and incubated for 20-22 hours at 37° C. After 20 hours, cells were stained with antibodies directed against CD8 and CD69 and specific recognition by TCR td T cells is measured by CD69 upregulation on a flow cytometer.

The presented data shows that the peptide sensitivity across a range of concentrations for the tested TCRs (FIGS. 19A-19B). In addition, it shows that the ability of the peptide to elicit a T cell response was not donor dependent.

### Example 14: Peptide Titration Assays for Both Class I and II-Restricted TCRs

Immortalized primary human B cells will be electroporated with graded concentration of a polypeptide of a uniform length (25mer) encoding a T cell epitope can be used in immuno-assays, involving the co-culture of these antigen expressing immortalized B cells with T cells engineered to express a defined MHC class II restricted TCR, to assess the sensitivity of the TCR.

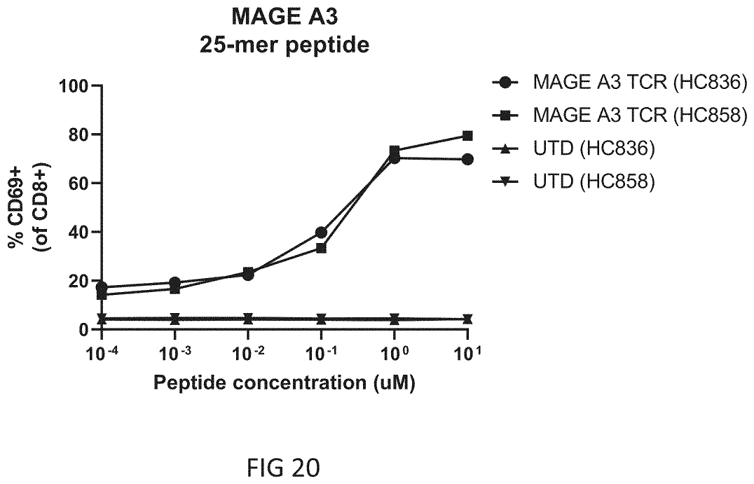

In an experiment conducted as in Example 13, a MAGEA3 TCR and corresponding mutated peptide were used. The data are shown in FIG. 20.

Taken together with Example 13, the data shows that peptide electroporation experiments using 25-mer peptides can be used to perform peptide titration assays for both class I and II-restricted TCRs.

### Example 15: Presentation of Long Peptides

Immortalized primary human B cells that are electroporated with a set of polypeptides of a uniform length (25mer) each encoding a different putative neo-antigen will be used to provide the mutated neo-antigen specificity of a TCR lead identified using a TCR discovery platform.

This example describes efficient presentation of long peptides using peptide electroporation of BCL6/xL-CD40L immortalized B cells, and utilization of these APCs in antigen screening.

### Example 15A: BCL6XL-CD40L Immortalized B Cells

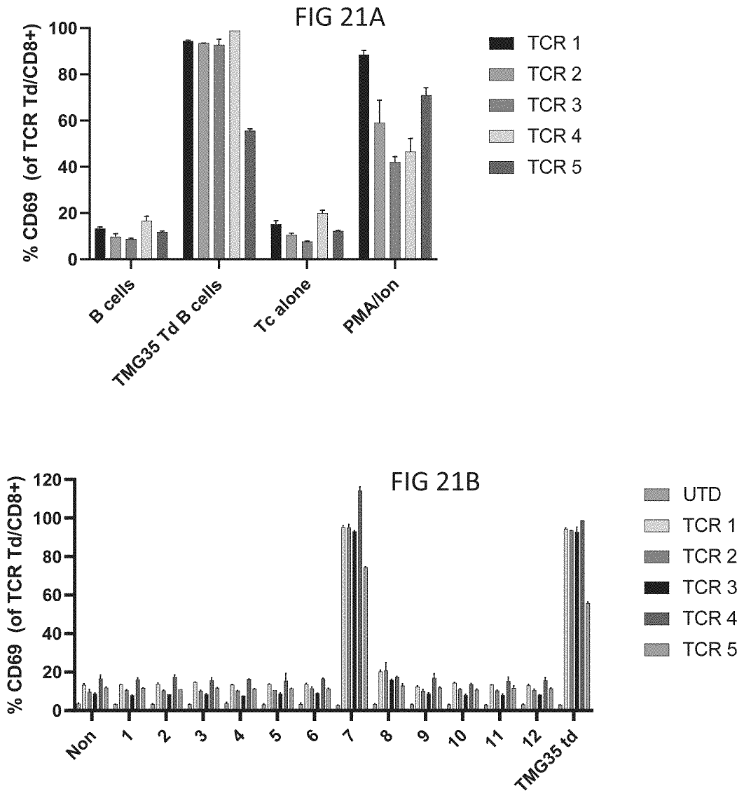

TCRs that recognize a TMG encoding twelve minigenes in a screen of a Basal Cell Carcinoma (BCC) patient using our TCR discovery platform were identified. Jurkat reporter T cell lines, each expressing one of the five of these TCR leads, were cocultured with autologous BCL6/xL-CD40L immortalized B cells expressing the single TMG construct in a single replicate. In order to generate TCR transduced Jurkat T cells, TCR-encoding plasmids were transfected into 293Vec-BaEV virus producer cells (Biovec) using Fugene transfection reagent and protocols known to the skilled artisan. The resulting retroviral virions were used to transduce a Jurkat reporter T cell line. The Jurkat reporter T cell line lacks endogenous TCR expression (generation of such a genetic knock-out being described for example in Mezzadra et al Nature 2017) and was modified to express human CD8α and CD8β after transduction with a CD8α-P2A-CD8β transgene using methods known to the skilled artisan. The use of murine TCR constant domain sequences as incorporated in the TCR transgene allows for the detection of TCR-modified Jurkat T cells by flow cytometry using a murine TCRβ constant domain specific antibody. Reporter TCR transduced T cells were seeded at 1e5 cells/mL 2 days prior to the experiment. The coculture was performed at a 1:1 effector:target cell ratio with 1e5 cells each per 96 well. After 16-18 hours, cells were stained with antibodies directed against CD8 and CD69 and specific recognition by TCR transduced T cells was measured by CD69 upregulation on a flow cytometer as shown in FIG. 21A. for identified TCR leads. Untransduced B cells (‘B cells’), medium without B cells (‘TC alone’) or medium supplemented with 50 ng/mL PMA and 1 uM lonomycin (‘PMA/Ion’) were used in the co-culture as controls for antigen presentation and/or T cell activation. After a (co-)culture with TCR-transduced effector cells, T cell activation was measured by CD69+ expression of the TCR transduced, CD8+ cells.

Subsequently, to determine the neo-antigen that is recognized by the five TCR leads, autologous BCL6/xL-CD40L immortalized B cells electroporated with single 25mer peptides that are encoded in the TMG were used to determine TCR specificity (FIG. 21B). Cells were electroporated as described previously. In short, 0.5e6 B cells per peptide were electroporated in 20 μL electroporation buffer, containing a 1 mM concentration of a single long peptide. After electroporation, cells were rested for 4 hours in 200 μL complete B cell medium (RPMI1640 medium supplemented with 10% (v/v) Fetal Calf Serum (FCS), 1% (v/v) Penicillin/Streptomycin (P/S), 1% (v/v) Sodium Pyruvate, 1% (v/v) Non-essential amino acids, 1% (v/v) Glutamax, 50 nM 2-Mercaptoethanol) resulting in a final peptide concentration of 100 uM. After the resting period, B cells were washed extensively, harvested, and distributed over multiple wells at 1e5 cells per well. Reporter Jurkat T cells lacking endogenous TCR expression were engineered to express the identified TCRs and were seeded at low density (100,000 cells per mL) 2 days before the assay. The coculture was performed at a 1:1 effector:target cell ratio with 1e5 cells each per 96 well. After 16-18 hours, cells were stained with antibodies directed against CD8 and CD69 and specific recognition by TCR-transduced T cells was measured by CD69 upregulation on a flow cytometer. Non-transduced T cells (‘UTD’), non-electroporated B cells (‘Non’) and the TMG that encodes all 12 epitopes (‘TMG35 td’) were used in the co-culture as controls for antigen presentation and/or T cell activation. These data show that all five TCR leads recognize peptide 7, albeit with different affinities (FIG. 21B).

### Example 15B: EBV Immortalized B Cells

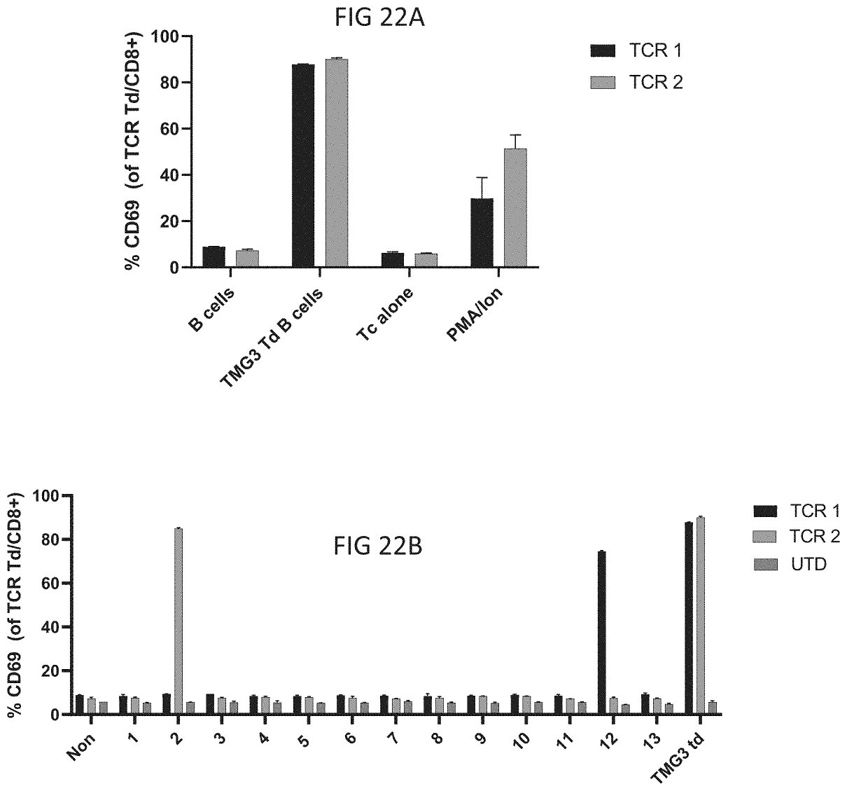

TCRs that recognize a TMG encoding nine minigenes in a screen on a Colorectal Carcinoma (CRC) patient using our TCR discovery platform were identified. Jurkat reporter T cell lines, each expressing one of the two TCR leads, were cocultured with EBV immortalized B cells expressing the single TMG construct in a single replicate. In order to generate TCR transduced Jurkat T cells, TCR-encoding plasmids were transfected into 293Vec-BaEV virus producer cells (Biovec) using Fugene transfection reagent and protocols known to the skilled artisan. The resulting retroviral virions were used to transduce a Jurkat reporter T cell line. The Jurkat reporter T cell line lacks endogenous TCR expression (generation of such a genetic knock-out being described for example in Mezzadra et al Nature 2017) and was modified to express human CD8α and CD8β after transduction with a CD8α-P2A-CD8β transgene using methods known to the skilled artisan. The use of murine TCR constant domain sequences as incorporated in the TCR transgene allows for the detection of TCR-modified Jurkat T cells by flow cytometry using a murine TCRβ constant domain specific antibody. Reporter TCR transduced T cells were seeded at 1e5 cells/mL 2 days prior to the experiment. The coculture was performed at a 1:1 effector:target cell ratio with 1e5 cells each per 96 well. After 16-18 hours, cells were stained with antibodies directed against CD8 and CD69 and specific recognition by TCR transduced T cells was measured by CD69 upregulation on a flow cytometer as shown in FIG. 22A for identified TCR leads. Untransduced B cells (‘B cells’), medium without B cells (‘TC alone’) or medium supplemented with 50 ng/mL PMA and 1 mM Ionomycin (‘PMA/Ion’) were used in the co-culture as controls for antigen presentation and/or T cell activation. After a (co-)culture with TCR-transduced effector cells, T cell activation was measured by CD69+ expression of the TCR transduced, CD8+ cells.

Subsequently, to determine the neo-antigen that is recognized by the two TCR leads, autologous EBV immortalized B cells electroporated with single 25mer peptides that are encoded in the TMG were used to determine TCR specificity (FIG. 22B). In this example, one of the minigenes was derived from a frameshift mutation, and five overlapping 25-mer peptides were used in this experiment as a result. Cells were electroporated as described previously. In short, 0.5e6 B cells per peptide were electroporated in 20 μL electroporation buffer, containing a 1 mM concentration of a single long peptide. After electroporation, cells were rested for 4 hours in 200 μL complete B cell medium (RPMI1640 medium supplemented with 10% (v/v) Fetal Calf Serum (FCS), 1% (v/v) Penicillin/Streptomycin (P/S), 1% (v/v) Sodium Pyruvate, 1% (v/v) Non-essential amino acids, 1% (v/v) Glutamax, 50 nM 2-Mercaptoethanol) resulting in a final peptide concentration of 100 uM. After the resting period, B cells were washed extensively, harvested, and distributed over multiple wells at 1e5 cells per well. Reporter Jurkat T cells lacking endogenous TCR expression were engineered to express the identified TCRs and were seeded at low density (100,000 cells per mL) 2 days before the assay. The coculture was performed at a 1:1 effector:target cell ratio with 1e5 cells each per 96 well. After 16-18 hours, cells were stained with antibodies directed against CD8 and CD69 and specific recognition by TCR-transduced T cells was measured by CD69 upregulation on a flow cytometer. Non-transduced T cells (‘UTD’), non-electroporated B cells (‘Non’) and the TMG that encodes all 13 epitopes (‘TMG3 td’) were used in the co-culture as controls for antigen presentation and/or T cell activation. These data show one TCR recognizes peptide 2 and another recognizes peptide 12 (FIG. 22B).

### Example 16: Presentation of Long Peptides Using Peptide Electroporation of BCL6/xL-CD40L Immortalized B Cells

Immortalized primary human B cells will be electroporated with a polypeptide of a uniform length (25mer) encoding a wild-type (non-mutated) counterpart of a neo-antigen can be used to assess the safety of a TCR lead identified using a TCR discovery platform.

This example describes efficient presentation of long peptides using peptide electroporation of BCL6XL/CD40L immortalized B cells, and utilization of these APCs in determining the ability of a TCR to distinguish between a wild-type antigen and a neo-antigen.

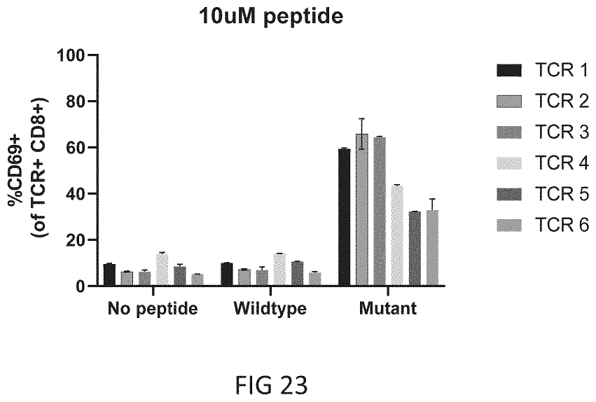

To determine mutation-specificity of the TCRs, BCL6/xL-CD40L immortalized B cells were electroporated with high concentrations (100 uM, final concentration after rest was 10 uM) of wildtype or mutant peptide as described in Example 15A. The wild-type antigen differed from the neo-antigen by a single amino acid substitution. The co-culture was performed as in Example 15 (with an additional identified TCR lead), in short; 1e5 electroporated B cells were cultured with 1e5 TCR-KO Jurkat T cells transduced with the corresponding TCRs, in a 1:1 effector:target cell ratio. After 16-18 hours, cells were stained with antibodies directed against CD8 and CD69 and specific recognition by TCR td T cells is measured by CD69 upregulation on a flow cytometer. These data show that all TCRs are mutation specific and do not recognize the wildtype peptide (FIG. 23).

### Example 17: Using APCs to Identify Neo-Antigen-Specific T Cell Receptor Genes from Non-Viable Tumor Biopsies

This example describes using the antigen-expressing cells described herein to identify neo-antigen-specific T cell receptor genes from non-viable tumor biopsies on a per patient basis.

A process to generate TCR libraries by combinatorial assembly of tumor-derived TCRα and TCRβ chains have been developed. These TCR chains are identified by bulk TCR chain sequencing of DNA or RNA isolated from tumor tissue. TCRαβ pairs are encoded as transgenes of approximate 1.8 kb and introduced into reporter cells. Stimulating by the antigen-expressing cells described herein, reporter cells expressing antigen-reactive TCRαβ combinations can be selected in a genetic variant library screening. Given the combinatorial assembly, each TCR variant can only be unambiguously identified by determining both TCRα and TCRβ variable sequences. Hence, transgenes encoded in reporter cells isolated during the genetic screen are recovered as PCR amplicons of approximate 1.5 kb, sequenced in full length using Oxford Nanopore sequencing and analyzed using a customized bioinformatic analysis pipeline. This process will identify neo-antigen-specific TCR leads that can be further evaluated for potential use in cancer therapy.

### Example 18: APC Cell Types

### Cell Types Used as Antigen-Presenting Cells

In principle, any cell can be engineered to serve as APC for TCR discovery. Primary human B cells are used here for two reasons: first, use of an autologous cell from the individual enables TCR discovery against all autologous HLA-alleles to facilitate fully personalized TCR discovery.

Immortalization of primary human B cells by either EBV infection or transduction with BCL-6/BCL-XL can be achieved. Thereby, large numbers of primary human B cells can be obtained in vitro by transduction of B cell subsets isolated from peripheral blood (see below). Large numbers of APCs are useful to perform the TCR discovery process with sufficient sensitivity. TCR libraries are screened in a functional genetic screening process. To date, typical TCR libraries include 10,000 TCR variants. In order to be able to detect TCRs present with a frequency of 1:10,000 each TCR variant should be represented on average 800× throughout the complete process. In order to screen such libraries, 4 replicate co-cultures each with 80 Million APCs with 80 Million reporter T cells are performed, thus requiring at least 320 Million APCs.

### Inducing, Enhancing or Maintaining Prolonged Survival of the Cell In Vitro

Prolonged survival of primary human B cells is achieved by retroviral transduction of isolated B cells with BCL-6 and BCL-XL. In brief, primary human pan-B cell subsets are isolated by negative magnetic bead-based selection from peripheral blood mononuclear cells (PBMC) and subsequently activated by co-culture with CD40L-expressing feeder cells in the presence of IL-2, IL-4 and IL-21. 72 hours after activation, B cells are retrovirally transduced with BCL-6, BCL-XL and CD40L. The data demonstrates that this process (1) is robust and can successfully be performed on minimal numbers of (long-time) cryo-preserved B cells (to date, it has been started with as low as 1×10⁵ isolated B cells obtained from PBMC material that was cryopreserved for 25+ years) and (2) leads to high cell numbers (at least 30-fold expansion within 28 days).

After transduction, B cells can be maintained in culture for at least three months. By introducing CD40L directly into B cells, modified B cells can be maintained in cell culture solely based on cytokine-supplemented media. Previously described methods require continuous supplementation of cultures with CD40L-expressing feeder cells approximately every three days. This novel modification enhances B cell maintenance in culture for prolonged time periods as well as automation and close-up of the culture process.

### Alternatives for the Present Example

All other methods commonly used to isolate B cells from a population of cells can be utilized, including flow cytometry sorting.

Instead of pan-B cell enrichment, one may enrich specific B cell subsets, including but not limited to, naïve B cells, memory B cells, pre-B cells, pro-B cells and immature B cells.

Primary human B cells can be also be immortalized by EBV infection.

The transgenes can be delivered by other methods that lead to stable transgene integration, including other viral gene delivery systems (Lentivirus) as well as non-viral gene delivery methods such as CRISPR/Cas9, TALEN and Transposon-based vectors. In some alternatives, the transgenes can be added via non-stable expression, e.g., from transfection/non-integrating viruses etc.

The transgenes may be delivered as individual transgenes or combined in polycistronic expression cassettes. In some alternatives, different cytokine support can be added to the B cells.

Incubating the Cell with at Least One Antigen Compound

An antigen is delivered into BCL-6/BCL-XL modified B cells by retroviral transduction. In brief, BCL-6/BCL-XL immortalized B cells are retrovirally transduced with one or more antigens. After transduction, antigen-expressing B cells can be detected by flow cytometry staining for a cell surface marker fused to the antigen and selected based on a puromycin resistance gene introduced into the B cell together with the antigen.

### Introducing at Least One Genetic Modification to Induce, Enhance, Maintain or Modify Antigen-Presentation by the Cell

The data demonstrates that expression of a CD40L transgene improves MHC-class II antigen presentation by the generated B cells. CD40L was initially introduced to eliminate the need for CD40L-expressing feeder cells for maintenance of BCL-6/BCL-XL immortalized B cells in cell culture.

One can also evaluate further modifications that specifically modify the antigen-presentation by the engineered B cells. Manipulation of the B cell will be used to direct TCR discovery, for example by focusing antigen-presentation on either the MHC-class I or class II presentation pathway (by genetic knock-out of essential pathway molecules) and adjusting T cell activation thresholds (by either genetic knock-out or overexpression of relevant co-stimulatory molecules). These strategies can provide alternative embodiments beyond CD40L transgene expression. Such alternatives are listed below.

Genetic Knock-out of B2M and/or TAP may be used to abrogate MHC-class I antigen presentation by the engineered B cells.

Genetic Knock-out of CIITA and/or CD74 (HLA-DR-antigens-associated invariant chain) may be used to abrogate MHC-class II antigen presentation by the engineered B cells.

Genetic Knock-out of specific HLA-alleles may be used to direct TCR discovery towards certain HLA-alleles.

Co-stimulatory molecules, including but not limited to, CD80, CD86, CD70, PD-L1 may either be knocked-out or overexpressed to control the threshold for T cell activation by the B cell.

Any desired genetic knock-out as outlined above may be achieved by site-specific integration of any transgene used to engineer the B cell (BCL-6; BCL-XL; antigen; CD40L) into the target locus provided that bi-allelic editing is highly efficient for the chosen target locus.

This example is the first description that a CD40L transgene expressed in B cells can enhance MHC-class II antigen presentation and lead to increased activation of MHC-class II restricted T cells. CD40L is normally expressed on T cells and studies aiming to study the effects of CD40 receptor stimulation typically use either co-culture with T cells or trimeric CD40L recombinant protein.

1. A method of engineering a cell for antigen presentation to T cells, the method comprising:
a) inducing, enhancing and/or maintaining prolonged survival of a cell in vitro;
b) incubating the cell with at least one antigen compound on a continuous basis; and
c) introducing at least one genetic modification within the cell to induce, enhance, maintain and/or modify antigen-presentation by the cell.

2. The method of claim 1, wherein the cell comprises a primary human B cell, optionally wherein the primary human B cell is autologous with respect to the T cells or to a TCR presented by the T cells.

3. The method of claim 2, wherein the primary human B cell is derived from peripheral blood.

4. The method of claim 2, wherein the primary human B cell is derived from a human tissue that contain B cells.

5. (canceled)

6. The method of claim 2, wherein the primary human B cell is obtained by differentiation of a precursor cell.

7. The method of claim 6, wherein the precursor cell comprises hematopoietic stem cells.

8. The method of claim 2, wherein the primary human B cell is obtained by differentiation of induced pluripotent stem cells (iPSC).

9. The method of claim 1, wherein the inducing, enhancing and/or maintaining prolonged survival of the cell comprises co-culturing the cell with CD40L-expressing feeder cells.

10. The method of claim 9, further comprising co-culturing the cell with IL-2, IL-4 and IL-21.

11. The method of claim 1, wherein the inducing, enhancing and/or maintaining prolonged survival of the cell comprises expressing BCL-6 and BCL2-like 1 genes in the cell.

12. The method of claim 1, wherein the inducing, enhancing and/or maintaining prolonged survival of the cell comprises infecting the cell with EBV.

13. The method of claim 10, further comprising expressing CD40L gene in the cell.

14-15. (canceled)

16. The method of claim 11, 13, or 11, wherein the genes are introduced into the cell through viral transduction, electroporation, squeezing, transfection, site-specific integration, transposons, CRISPR/Cas9, or TALEN.

17. The method of claim 1, wherein the prolonged survival of the cell is at least three months in cell culture in vitro.

18. The method of claim 1, wherein the at least one antigen compound is encoded by one transgene encoding between one and forty polypeptides.

19. The method of claim 18, wherein each polypeptide comprises at least eight or nine amino acids.

20. The method of claim 1, wherein the at least one antigen compound is expressed in the cell.

21. The method of claim 1, wherein the at least one antigen compound is transiently expressed in the cell.

22. An engineered cell, wherein the cell:
a) has been adjusted to induce, enhance and/or maintain its survival in vitro;
b) has been incubated with at least one antigen compound; and
c) has at least one genetic modification to induce, enhance, maintain or modify antigen-presentation by the cell.

23. The engineered cell of claim 22, wherein adjusted to induce, enhance and/or maintain its survival in vitro comprises expressing BCL-6 and BCL2-like 1 genes in the cell.

24. The engineered cell of claim 22, wherein adjusted to induce, enhance and/or maintain its survival in vitro comprises infecting the cell with EBV.

25. The engineered cell of claim 23, further comprising expressing CD40L gene in the cell.

26-27. (canceled)

28. The engineered cell of claim 22, wherein the at least one antigen compound is encoded by at least one transgene encoding between one and forty polypeptides.

29. The engineered cell of claim 28, wherein each polypeptide comprises at least eight amino acids.

30. The engineered cell of claim 22, wherein the at least one antigen compound is stably expressed in the cell.

31. (canceled)

32. An engineered cell comprising:
a) a nucleotide sequence for expression of a survival factor;
b) a nucleotide sequence for expression of at least one transgene encoding an antigen; and
c) a nucleotide sequence for expression of CD40L.

33. The engineered cell of claim 32, wherein the survival factor comprises BCL-6 and/or BCL-XL.

34-36. (canceled)

37. A primary human B cell to present antigen to T cells, the cell comprising:
a) a nucleotide sequence providing stable expression of BCL-6 and BCL-XL;
b) a nucleotide sequence providing stable expression of between one and forty polypeptides, each polypeptide encoding at least eight amino acids, and each polypeptide being an antigen; and
c) a nucleotide sequence providing stable expression of CD40L.

38. A method of antigen presentation, the method comprising:
inducing, enhancing and/or maintaining prolonged survival of a cell in vitro;
incubating the cell with at least one antigen compound on a continuous basis; and
introducing at least one genetic modification within the cell to induce, enhance, maintain and/or modify antigen-presentation by the cell.

39-49. (canceled)

50. A culturing mix comprising engineered cells according to claim 22 and a culturing medium.

51-107. (canceled)


SEQUENCE LISTING (10 sequences)

SEQ ID NO: 1 - protein, 1223 aa, Artificial Sequence
  Synthetic Polypeptide
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKACGSGEGRGSLLTCGDVEENPGPMSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRKEFGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 2 - protein, 467 aa, Artificial Sequence
  Synthetic Polypeptide
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVGSGATNFSLLKQAGDVEENPGPMRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 3 - protein, 14 aa, Artificial Sequence
  Synthetic Polypeptide
SLLMWITQCFLPVF

SEQ ID NO: 4 - protein, 9 aa, Artificial Sequence
  Synthetic Polypeptide
SLLMWITQC

SEQ ID NO: 5 - protein, 10 aa, Artificial Sequence
  Synthetic Polypeptide
ALLETPSLLL

SEQ ID NO: 6 - protein, 25 aa, Artificial Sequence
  Synthetic Polypeptide
FSWNVKAALLETPSLLLAKVGIALK

SEQ ID NO: 7 - protein, 25 aa, Artificial Sequence
  Synthetic Polypeptide
WQAGILARNLVPMVATVQGQNLKYQ

SEQ ID NO: 8 - protein, 25 aa, Artificial Sequence
  Synthetic Polypeptide
ILGDPKKLLTQHFVQENYLEYRQVP

SEQ ID NO: 9 - protein, 261 aa, Artificial Sequence
  Synthetic Polypeptide
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 10 - protein, 261 aa, Artificial Sequence; identical to SEQ ID NO: 9
  Synthetic Polypeptide